SEQUENCE LISTING (134 sequences)

SEQ ID NO: 1 - protein, 394 aa, Human alphaherpesvirus 1
MGGTAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 2 - protein, 369 aa, Human alphaherpesvirus 1
KYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 3 - protein, 33 aa, Human alphaherpesvirus 1
DASLKMADPNRFRGKDLPVLDQLTDPPGVRRVY

SEQ ID NO: 4 - protein, 16 aa, Saccharomyces cerevisiae
KNYHLENEVARLKKLV

SEQ ID NO: 5 - protein, 30 aa, Artificial Sequence
  Description of Artificial Sequence Synthetic polypeptide
TSGSKNYHLENEVARLKKLVGSGGGGSGNS

SEQ ID NO: 6 - protein, 43 aa, Artificial Sequence
  Description of Artificial Sequence Synthetic polypeptide
LEIRAAFLRQRNTALRTEVAELEQEVQRLENEVSQYETRYGPL

SEQ ID NO: 7 - DNA, 129 bp, Artificial Sequence
  Description of Artificial Sequence Synthetic polynucleotide
ctggaaatcagagccgctttcctgagacagcggaacaccgccctgcggaccgaggtggccgagctggaacaggaggtgcagagactggaaaacgaggtgtcccaatacgagacaagatacggccctctg

SEQ ID NO: 8 - protein, 63 aa, Artificial Sequence
  Description of Artificial Sequence Synthetic polypeptide
GTLEIRAAFLRQRNTALRTEVAELEQEVQRLENEVSQYETRYGPLGGGGSGGGGSGGGGSGNS

SEQ ID NO: 9 - DNA, 189 bp, Artificial Sequence
  Description of Artificial Sequence Synthetic polynucleotide
ggtaccctggaaatcagagccgctttcctgagacagcggaacaccgccctgcggaccgaggtggccgagctggaacaggaggtgcagagactggaaaacgaggtgtcccaatacgagacaagatacggccctctgggcggcggcggaagcggcggaggcggcagcggcggcggcggatctgggaattct

SEQ ID NO: 10 - protein, 43 aa, Artificial Sequence
  Description of Artificial Sequence Synthetic polypeptide
LEIEAAFLERENTALETRVAELRQRVQRLRNRVSQYRTRYGPL

SEQ ID NO: 11 - DNA, 129 bp, Artificial Sequence
  Description of Artificial Sequence Synthetic polynucleotide
ctggaaatcgaggccgccttcctggaacgggaaaacaccgccctggagacaagagtcgccgagctgagacagcgggtgcagagactgcggaatagagtgtcccaataccgcaccagatacggccctctg

SEQ ID NO: 12 - protein, 11 aa, Homo sapiens
SKPLPEVTDEY

SEQ ID NO: 13 - protein, 22 aa, Artificial Sequence
  Description of Artificial Sequence Synthetic peptide
GTGSKPLPEVTDEYGGGGSGNS

SEQ ID NO: 14 - DNA, 63 bp, Artificial Sequence
  Description of Artificial Sequence Synthetic oligonucleotide
accggcagcaagcccctgcccgaggtgaccgacgagtacggcggcggcggctccgggaattct

SEQ ID NO: 15 - protein, 20 aa, Artificial Sequence
  Description of Artificial Sequence Synthetic peptide
GGGGSGGGGSGGGGSGGGGS

SEQ ID NO: 16 - protein, 8 aa, Artificial Sequence
  Description of Artificial Sequence Synthetic peptide
GFSLTDYG

SEQ ID NO: 17 - protein, 7 aa, Artificial Sequence
  Description of Artificial Sequence Synthetic peptide
IWGDGIT

SEQ ID NO: 18 - protein, 7 aa, Artificial Sequence
  Description of Artificial Sequence Synthetic peptide
VTGLFDY

SEQ ID NO: 19 - protein, 9 aa, Artificial Sequence
  Description of Artificial Sequence Synthetic peptide
TGAVTTSNY

SEQ ID NO: 20 - protein, 3 aa, Artificial Sequence
  Description of Artificial Sequence Synthetic peptide
GTN

SEQ ID NO: 21 - protein, 9 aa, Artificial Sequence
  Description of Artificial Sequence Synthetic peptide
ALWYSNHWV

SEQ ID NO: 22 - protein, 113 aa, Artificial Sequence
  Description of Artificial Sequence Synthetic polypeptide
DVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSS

SEQ ID NO: 23 - protein, 109 aa, Artificial Sequence
  Description of Artificial Sequence Synthetic polypeptide
DAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVL

SEQ ID NO: 24 - protein, 242 aa, Artificial Sequence
  Description of Artificial Sequence Synthetic polypeptide
DVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSGGGGSGGGGSGGGGSGGGGSDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVL

SEQ ID NO: 25 - protein, 242 aa, Artificial Sequence
  Description of Artificial Sequence Synthetic polypeptide
DAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSS

SEQ ID NO: 26 - protein, 10 aa, Artificial Sequence
  Description of Artificial Sequence Synthetic peptide
GYTFTHYYIY

SEQ ID NO: 27 - protein, 14 aa, Artificial Sequence
  Description of Artificial Sequence Synthetic peptide
WMGGVNPSNGGTHF

SEQ ID NO: 28 - protein, 12 aa, Artificial Sequence
  Description of Artificial Sequence Synthetic peptide
RSEYDYGLGFAY

SEQ ID NO: 29 - protein, 14 aa, Artificial Sequence
  Description of Artificial Sequence Synthetic peptide
QSLLNSRTPKNYLA

SEQ ID NO: 30 - protein, 11 aa, Artificial Sequence
  Description of Artificial Sequence Synthetic peptide
LLIYWASTRKS

SEQ ID NO: 31 - protein, 7 aa, Artificial Sequence
  Description of Artificial Sequence Synthetic peptide
KQSYNLL

SEQ ID NO: 32 - protein, 120 aa, Artificial Sequence
  Description of Artificial Sequence Synthetic polypeptide
QVQLVQSGAEVKKPGASVKVSCKASGYTFTHYYIYWVRQAPGQGLEWMGGVNPSNGGTHFNEKFKSRVTMTRDTSISTAYMELSRLRSDDTAVYYCARSEYDYGLGFAYWGQGTLVTVSS

SEQ ID NO: 33 - protein, 112 aa, Artificial Sequence
  Description of Artificial Sequence Synthetic polypeptide
DIVMTQSPDSLAVSLGERATINCKSSQSLLNSRTPKNYLAWYQQKPGQPPKLLIYWASTRKSGVPDRFSGSGSGTDFTLTISSLQAEDVAVYYCKQSYNLLTFGGGTKVEIK

SEQ ID NO: 34 - protein, 252 aa, Artificial Sequence
  Description of Artificial Sequence Synthetic polypeptide
QVQLVQSGAEVKKPGASVKVSCKASGYTFTHYYIYWVRQAPGQGLEWMGGVNPSNGGTHFNEKFKSRVTMTRDTSISTAYMELSRLRSDDTAVYYCARSEYDYGLGFAYWGQGTLVTVSSGGSEGKSSGSGSESKSTGGSDIVMTQSPDSLAVSLGERATINCKSSQSLLNSRTPKNYLAWYQQKPGQPPKLLIYWASTRKSGVPDRFSGSGSGTDFTLTISSLQAEDVAVYYCKQSYNLLTFGGGTKVEIK

SEQ ID NO: 35 - protein, 8 aa, Artificial Sequence
  Description of Artificial Sequence Synthetic peptide
GSTFSINA

SEQ ID NO: 36 - protein, 7 aa, Artificial Sequence
  Description of Artificial Sequence Synthetic peptide
LSSGGSK

SEQ ID NO: 37 - protein, 19 aa, Artificial Sequence
  Description of Artificial Sequence Synthetic peptide
NAEIYYSDGVDDGYRGMDY

SEQ ID NO: 38 - protein, 125 aa, Artificial Sequence
  Description of Artificial Sequence Synthetic polypeptide
QLQLVESGGGLVHAGGSLRLSCAASGSTFSINAIGWYRQAPGKQRELVAALSSGGSKNYADSVKGRFTISRDNAKNTVYLQMNRLKPEDTAVYYCNAEIYYSDGVDDGYRGMDYWGKGTQVTVSS

SEQ ID NO: 39 - protein, 8 aa, Artificial Sequence
  Description of Artificial Sequence Synthetic peptide
GPPLSSYA

SEQ ID NO: 40 - protein, 8 aa, Artificial Sequence
  Description of Artificial Sequence Synthetic peptide
ISWSGSNT

SEQ ID NO: 41 - protein, 20 aa, Artificial Sequence
  Description of Artificial Sequence Synthetic peptide
AADRRGGPLSDYEWEDEYAD

SEQ ID NO: 42 - protein, 129 aa, Artificial Sequence
  Description of Artificial Sequence Synthetic polypeptide
EVQVVESGGGLVQTGGSLRLSCAASGPPLSSYAVAWFRQTPGKEREFVAAISWSGSNTYYADSVKGRFTISKDNAKNTVLVYLQMNSLKPEDTAVYYCAADRRGGPLSDYEWEDEYADWGQGTQVTVSS

SEQ ID NO: 43 - protein, 8 aa, Artificial Sequence
  Description of Artificial Sequence Synthetic peptide
GFTFSFYN

SEQ ID NO: 44 - protein, 8 aa, Artificial Sequence
  Description of Artificial Sequence Synthetic peptide
ISTSSSTI

SEQ ID NO: 45 - protein, 21 aa, Artificial Sequence
  Description of Artificial Sequence Synthetic peptide
AREGSYYDSSGYPYYYYDMDV

SEQ ID NO: 46 - protein, 9 aa, Artificial Sequence
  Description of Artificial Sequence Synthetic peptide
SSNIGAGYD

SEQ ID NO: 47 - protein, 3 aa, Artificial Sequence
  Description of Artificial Sequence Synthetic peptide
GNT

SEQ ID NO: 48 - protein, 14 aa, Artificial Sequence
  Description of Artificial Sequence Synthetic peptide
QSYDSSLSGTPYVV

SEQ ID NO: 49 - protein, 128 aa, Artificial Sequence
  Description of Artificial Sequence Synthetic polypeptide
EVQLVESGGGLVQPGGSLRLSCAASGFTFSFYNMNWVRQAPGKGLEWISYISTSSSTIYYADSVKGRFTISRDNAKNSLYLQMNSLRDEDTAVYYCAREGSYYDSSGYPYYYYDMDVWGQGTTVTVSS

SEQ ID NO: 50 - protein, 114 aa, Artificial Sequence
  Description of Artificial Sequence Synthetic polypeptide
QSVLTQPPSVSGAPGQRVTISCTGSSSNIGAGYDVHWYQQLPGTAPKLLIYGNTNRPSGVPDRFSGSKSGTSASLAITGLQAEDEADYYCQSYDSSLSGTPYVVFGGGTKLTVL

SEQ ID NO: 51 - protein, 262 aa, Artificial Sequence
  Description of Artificial Sequence Synthetic polypeptide
EVQLVESGGGLVQPGGSLRLSCAASGFTFSFYNMNWVRQAPGKGLEWISYISTSSSTIYYADSVKGRFTISRDNAKNSLYLQMNSLRDEDTAVYYCAREGSYYDSSGYPYYYYDMDVWGQGTTVTVSSGGSEGKSSGSGSESKSTGGSQSVLTQPPSVSGAPGQRVTISCTGSSSNIGAGYDVHWYQQLPGTAPKLLIYGNTNRPSGVPDRFSGSKSGTSASLAITGLQAEDEADYYCQSYDSSLSGTPYVVFGGGTKLTVL

SEQ ID NO: 52 - protein, 262 aa, Artificial Sequence
  Description of Artificial Sequence Synthetic polypeptide
QSVLTQPPSVSGAPGQRVTISCTGSSSNIGAGYDVHWYQQLPGTAPKLLIYGNTNRPSGVPDRFSGSKSGTSASLAITGLQAEDEADYYCQSYDSSLSGTPYVVFGGGTKLTVLGGSEGKSSGSGSESKSTGGSEVQLVESGGGLVQPGGSLRLSCAASGFTFSFYNMNWVRQAPGKGLEWISYISTSSSTIYYADSVKGRFTISRDNAKNSLYLQMNSLRDEDTAVYYCAREGSYYDSSGYPYYYYDMDVWGQGTTVTVSS

SEQ ID NO: 53 - protein, 8 aa, Artificial Sequence
  Description of Artificial Sequence Synthetic peptide
GFTFSSYS

SEQ ID NO: 54 - protein, 8 aa, Artificial Sequence
  Description of Artificial Sequence Synthetic peptide
ISGSGGFT

SEQ ID NO: 55 - protein, 11 aa, Artificial Sequence
  Description of Artificial Sequence Synthetic peptide
ARMPLNSPHDY

SEQ ID NO: 56 - protein, 6 aa, Artificial Sequence
  Description of Artificial Sequence Synthetic peptide
QGIRND

SEQ ID NO: 57 - protein, 3 aa, Artificial Sequence
  Description of Artificial Sequence Synthetic peptide
AAS

SEQ ID NO: 58 - protein, 9 aa, Artificial Sequence
  Description of Artificial Sequence Synthetic peptide
LQDYNYPLT

SEQ ID NO: 59 - protein, 118 aa, Artificial Sequence
  Description of Artificial Sequence Synthetic polypeptide
EVQLLESGGGLVQPGGSLRLSCAASGFTFSSYSMSWVRQAPGKGLEWVSVISGSGGFTDYADSVKGRFTISRDNSKNTLYLQMNSLRAEDTAVYYCARMPLNSPHDYWGQGTLVTVSS

SEQ ID NO: 60 - protein, 107 aa, Artificial Sequence
  Description of Artificial Sequence Synthetic polypeptide
DIQMTQSPSSLSASVGDRVTITCRASQGIRNDLGWYQQKPGKAPKLLIYAASSLQSGVPSRFSGSGSGTDFTLTISSLQPEDFATYYCLQDYNYPLTFGGGTKVEIK

SEQ ID NO: 61 - protein, 8 aa, Artificial Sequence
  Description of Artificial Sequence Synthetic peptide
GVSISSYF

SEQ ID NO: 62 - protein, 7 aa, Artificial Sequence
  Description of Artificial Sequence Synthetic peptide
ISTSGST

SEQ ID NO: 63 - protein, 9 aa, Artificial Sequence
  Description of Artificial Sequence Synthetic peptide
VRDWTGFDY

SEQ ID NO: 64 - protein, 9 aa, Artificial Sequence
  Description of Artificial Sequence Synthetic peptide
SSDVGSYNL

SEQ ID NO: 65 - protein, 3 aa, Artificial Sequence
  Description of Artificial Sequence Synthetic peptide
EGS

SEQ ID NO: 66 - protein, 10 aa, Artificial Sequence
  Description of Artificial Sequence Synthetic peptide
SSYAGSSTYV

SEQ ID NO: 67 - protein, 115 aa, Artificial Sequence
  Description of Artificial Sequence Synthetic polypeptide
QVQLQESGPGLVKPSETLSLTCTVSGVSISSYFWSWLRQPAGKGLQWIGRISTSGSTNHNPSLKSRVIMSVDTSKNQFSLKLSSVTAADTAVYYCVRDWTGFDYWGQGTLVTVSS

SEQ ID NO: 68 - protein, 110 aa, Artificial Sequence
  Description of Artificial Sequence Synthetic polypeptide
SYELTQPASVSGSPGQSITISCIGTSSDVGSYNLVSWYQQHPGKVPKLMIYEGSKRPSGVSNRFSGSKSGNTASLTISGLQAEDEADYYCSSYAGSSTYVFGTGTKVTVL

SEQ ID NO: 69 - protein, 245 aa, Artificial Sequence
  Description of Artificial Sequence Synthetic polypeptide
QVQLQESGPGLVKPSETLSLTCTVSGVSISSYFWSWLRQPAGKGLQWIGRISTSGSTNHNPSLKSRVIMSVDTSKNQFSLKLSSVTAADTAVYYCVRDWTGFDYWGQGTLVTVSSGGSEGKSSGSGSESKSTGGSSYELTQPASVSGSPGQSITISCIGTSSDVGSYNLVSWYQQHPGKVPKLMIYEGSKRPSGVSNRFSGSKSGNTASLTISGLQAEDEADYYCSSYAGSSTYVFGTGTKVTVL

SEQ ID NO: 70 - protein, 245 aa, Artificial Sequence
  Description of Artificial Sequence Synthetic polypeptide
DIQMTQSPSSLSASVGDRVTITCRASQGIRNDLGWYQQKPGKAPKLLIYAASSLQSGVPSRFSGSGSGTDFTLTISSLQPEDFATYYCLQDYNYPLTFGGGTKVEIKGGSEGKSSGSGSESKSTGGSEVQLLESGGGLVQPGGSLRLSCAASGFTFSSYSMSWVRQAPGKGLEWVSVISGSGGFTDYADSVKGRFTISRDNSKNTLYLQMNSLRAEDTAVYYCARMPLNSPHDYWGQGTLVTVSS

SEQ ID NO: 71 - protein, 245 aa, Artificial Sequence
  Description of Artificial Sequence Synthetic polypeptide
SYELTQPASVSGSPGQSITISCIGTSSDVGSYNLVSWYQQHPGKVPKLMIYEGSKRPSGVSNRFSGSKSGNTASLTISGLQAEDEADYYCSSYAGSSTYVFGTGTKVTVLGGSEGKSSGSGSESKSTGGSQVQLQESGPGLVKPSETLSLTCTVSGVSISSYFWSWLRQPAGKGLQWIGRISTSGSTNHNPSLKSRVIMSVDTSKNQFSLKLSSVTAADTAVYYCVRDWTGFDYWGQGTLVTVSS

SEQ ID NO: 72 - protein, 9 aa, Artificial Sequence
  Description of Artificial Sequence Synthetic peptide
GNSITSDYA

SEQ ID NO: 73 - protein, 7 aa, Artificial Sequence
  Description of Artificial Sequence Synthetic peptide
ISYSGST

SEQ ID NO: 74 - protein, 10 aa, Artificial Sequence
  Description of Artificial Sequence Synthetic peptide
ATGYYYGSGF

SEQ ID NO: 75 - protein, 10 aa, Artificial Sequence
  Description of Artificial Sequence Synthetic peptide
ESVEYFGTSL

SEQ ID NO: 76 - protein, 3 aa, Artificial Sequence
  Description of Artificial Sequence Synthetic peptide
AAS

SEQ ID NO: 77 - protein, 9 aa, Artificial Sequence
  Description of Artificial Sequence Synthetic peptide
QQTRKVPYT

SEQ ID NO: 78 - protein, 117 aa, Artificial Sequence
  Description of Artificial Sequence Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 79 - protein, 107 aa, Artificial Sequence
  Description of Artificial Sequence Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTK

SEQ ID NO: 80 - protein, 117 aa, Artificial Sequence
  Description of Artificial Sequence Synthetic polypeptide
QVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSS

SEQ ID NO: 81 - protein, 111 aa, Artificial Sequence
  Description of Artificial Sequence Synthetic polypeptide
EIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIK

SEQ ID NO: 82 - protein, 244 aa, Artificial Sequence
  Description of Artificial Sequence Synthetic polypeptide
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSGTEGKSSGSGSESKSTDIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIK

SEQ ID NO: 83 - protein, 248 aa, Artificial Sequence
  Description of Artificial Sequence Synthetic polypeptide
QVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIK

SEQ ID NO: 84 - protein, 244 aa, Artificial Sequence
  Description of Artificial Sequence Synthetic polypeptide
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKGTEGKSSGSGSESKSTQVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 85 - protein, 248 aa, Artificial Sequence
  Description of Artificial Sequence Synthetic polypeptide
EIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSS

SEQ ID NO: 86 - protein, 158 aa, Artificial Sequence
  Description of Artificial Sequence Synthetic polypeptide
GSDLGKKLLEAARAGQDDEVRILMANGADVNASDRYGRTPLHLAAFNGHLEIVEVLLKNGADVNAKDKIGNTPLHLAANHGHLEIVEVLLKYGAVVNATDWLGVTPLHLAAVFGHLEIVEVLLKYGADVNAQDKFGKTAFDISIDNGNEDLAEILQKL

SEQ ID NO: 87 - protein, 429 aa, Artificial Sequence
  Description of Artificial Sequence Synthetic polypeptide
METDTLLLWVLLLWVPGSTGDQLQLVESGGGLVHAGGSLRLSCAASGSTFSINAIGWYRQAPGKQRELVAALSSGGSKNYADSVKGRFTISRDNAKNTVYLQMNRLKPEDTAVYYCNAEIYYSDGVDDGYRGMDYWGKGTQVTVSSNSGGGGSDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSGGGGSLESRGPFEQKLISEEDLNMHTGHHHHHH

SEQ ID NO: 88 - DNA, 1290 bp, Artificial Sequence
  Description of Artificial Sequence Synthetic polynucleotide
atggagaccgacacactgctgctgtgggtgctgctgctgtgggtgcccggctctacaggcgatcagctgcagctggtggagagcggaggaggcctggtgcacgcaggaggcagcctgaggctgtcctgcgcagcatctggcagcaccttcagcatcaacgcaatcggatggtacaggcaggcacctggcaagcagagggagctggtggccgccctgagctccggcggcagcaagaattacgccgactccgtgaagggccggtttacaatcagcagagataacgccaagaataccgtgtatctgcagatgaacaggctgaagccagaggacaccgccgtgtactattgcaatgccgagatctactattccgacggagtggacgatggctaccgcggaatggattattggggcaagggcacacaggtgaccgtgtcttcgaattctggaggaggaggctctgacgcagtggtgacacaggagagcgccctgaccacatcccctggagagaccgtgacactgacctgtcgctcctctaccggcgccgtgaccacatctaattatgccagctgggtgcaggagaagccagatcacctgttcacaggcctgatcggaggcaccaacaatagggcaccaggcgtgcctgcaagattttccggctctctgatcggcgacaaggccgccctgacaatcaccggagcacagaccgaggatgaggccatctacttctgcgccctgtggtatagcaaccactgggtgtttggcggcggcacaaagctgaccgtgctgggaggaggaggaggctctggaggaggaggcagcggcggcggcggctccggcggcggcggctctgacgtgcagctgcagcagtccggaccaggcctggtggcacccagccagtccctgtctatcacatgtaccgtgtctggcttcagcctgaccgattacggagtgaactgggtgcggcagtccccaggcaagggactggagtggctgggcgtgatctggggcgacggcatcacagattataattctgccctgaagtcccggctgtctgtgaccaaggataacagcaagtcccaggtgttcctgaagatgaatagcctgcagtccggcgactctgccagatactattgcgtgacaggcctgtttgattactggggccagggcaccacactgaccgtgagctccggaggaggaggctccctcgagtctagagggcccttcgaacaaaaactcatctcagaagaggatctgaatatgcataccggtcatcatcaccatcaccattga

SEQ ID NO: 89 - protein, 435 aa, Artificial Sequence
  Description of Artificial Sequence Synthetic polypeptide
METDTLLLWVLLLWVPGSTGDTGDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSNSGGGGSEVQVVESGGGLVQTGGSLRLSCAASGPPLSSYAVAWFRQTPGKEREFVAAISWSGSNTYYADSVKGRFTISKDNAKNTVLVYLQMNSLKPEDTAVYYCAADRRGGPLSDYEWEDEYADWGQGTQVTVSSGGGGSLESRGPFEQKLISEEDLNMHTGHHHHHH

SEQ ID NO: 90 - DNA, 1308 bp, Artificial Sequence
  Description of Artificial Sequence Synthetic polynucleotide
atggagaccgacacactgctgctgtgggtgctgctgctgtgggtgccaggcagcacaggcgacaccggcgatgcagtggtgacacaggagagcgccctgaccacatccccaggagagaccgtgacactgacctgcaggagctccaccggagcagtgaccacatccaactacgcctcttgggtgcaggagaagcccgatcacctgttcacaggcctgatcggcggcaccaacaatagggcaccaggcgtgcccgcacgcttttctggcagcctgatcggcgacaaggccgccctgacaatcaccggagcacagacagaggatgaggccatctacttctgcgccctgtggtatagcaatcactgggtgtttggcggcggcacaaagctgaccgtgctgggaggaggaggaggctctggaggaggaggcagcggcggcggcggctccggcggcggcggctctgacgtgcagctgcagcagtccggacctggcctggtggcaccatcccagtctctgagcatcacatgtaccgtgagcggcttctccctgaccgattacggagtgaactgggtgcggcagtcccctggcaagggactggagtggctgggcgtgatctggggcgacggcatcacagattataattctgccctgaagtctaggctgagcgtgaccaaggacaactccaagtctcaggtgttcctgaagatgaacagcctgcagtctggcgacagcgcccgctactattgcgtgacaggcctgtttgattactggggccagggcaccacactgaccgtgtcttcgaattctggaggaggaggctccgaggtgcaggtggtggagagcggaggaggcctggtgcagaccggaggcagcctgcggctgtcctgtgcagcatctggaccacctctgtcctcttatgcagtggcatggttcaggcagacaccaggcaaggagagagagtttgtggccgccatcagctggtccggctctaacacctactatgccgactctgtgaagggccggttcaccatcagcaaggataacgccaagaataccgtgctggtgtacctgcagatgaatagcctgaagcccgaggataccgccgtgtactattgtgcagcagacaggagaggaggacctctgtccgattacgagtgggaggacgagtatgccgattggggccagggcacacaggtgaccgtgagctccggaggaggaggctccctcgagtctagagggcccttcgaacaaaaactcatctcagaagaggatctgaatatgcataccggtcatcatcaccatcaccattga

SEQ ID NO: 91 - protein, 561 aa, Artificial Sequence
  Description of Artificial Sequence Synthetic polypeptide
METDTLLLWVLLLWVPGSTGDTGQSVLTQPPSVSGAPGQRVTISCTGSSSNIGAGYDVHWYQQLPGTAPKLLIYGNTNRPSGVPDRFSGSKSGTSASLAITGLQAEDEADYYCQSYDSSLSGTPYVVFGGGTKLTVLGGSEGKSSGSGSESKSTGGSEVQLVESGGGLVQPGGSLRLSCAASGFTFSFYNMNWVRQAPGKGLEWISYISTSSSTIYYADSVKGRFTISRDNAKNSLYLQMNSLRDEDTAVYYCAREGSYYDSSGYPYYYYDMDVWGQGTTVTVSSGGGGSDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSLESRGPFEQKLISEEDLNMHTGHHHHHH

SEQ ID NO: 92 - DNA, 1686 bp, Artificial Sequence
  Description of Artificial Sequence Synthetic polynucleotide
atggagaccgacacactgctgctgtgggtgctgctgctgtgggtgcctggctccacaggcgataccggacagtctgtgctgacccagccacctagcgtgtccggagcaccaggccagcgggtgacaatctcctgcaccggcagctcctctaacatcggcgccggctacgacgtgcactggtatcagcagctgcctggcacagccccaaagctgctgatctacggcaacaccaataggcccagcggcgtgcctgatcgcttttctggcagcaagtccggcacatctgccagcctggcaatcaccggactgcaggcagaggacgaggccgattactattgccagtcttacgacagctccctgagcggcacaccttatgtggtgttcggaggaggcacaaagctgaccgtgctgggaggcagcgagggcaagtctagcggctccggctctgagagcaagtccaccggaggcagcgaggtgcagctggtggagtccggaggaggcctggtgcagccaggaggcagcctgcggctgtcctgtgccgcctctggcttcaccttttccttctacaacatgaattgggtgagacaggcacctggcaagggcctggagtggatcagctatatctccacatcctctagcaccatctactatgccgacagcgtgaagggccggtttacaatcagccgggacaacgccaagaatagcctgtacctgcagatgaacagcctgagggacgaggataccgccgtgtactattgcgcccgcgagggctcctactatgactcctctggctatccatactattactatgacatggacgtgtggggccagggcaccacagtgacagtgagctccggcggaggaggcagcgatgcagtggtgacccaggagtctgccctgaccacaagcccaggcgagaccgtgacactgacctgtcggtctagcaccggcgccgtgaccacaagcaactacgcctcctgggtgcaggagaagcccgaccacctgtttacaggcctgatcggaggcaccaacaatagggcaccaggcgtgcccgcaagattctctggcagcctgatcggcgacaaggccgccctgacaatcaccggagcacagaccgaggatgaggccatctacttttgcgccctgtggtattccaatcactgggtgttcggcggcggcacaaagctgaccgtgctgggtggaggaggaggctccggaggaggaggctctggcggcggcggcagcggaggcggcggctccgacgtgcagctgcagcagagcggaccaggcctggtggcaccatcccagtctctgagcatcacatgtaccgtgtctggcttcagcctgaccgattacggcgtgaactgggtgagacagtctccaggcaagggcctggagtggctgggcgtgatctggggcgacggcatcacagattataatagcgccctgaagtccaggctgtctgtgaccaaggataactccaagtctcaggtgtttctgaagatgaatagcctgcagtccggcgactctgcccgctactattgcgtgacaggcctgttcgattactggggacagggcaccacactgaccgtgtcctctctcgagtctagagggcccttcgaacaaaaactcatctcagaagaggatctgaatatgcataccggtcatcatcaccatcaccattga

SEQ ID NO: 93 - protein, 561 aa, Artificial Sequence
  Description of Artificial Sequence Synthetic polypeptide
METDTLLLWVLLLWVPGSTGDTGEVQLVESGGGLVQPGGSLRLSCAASGFTFSFYNMNWVRQAPGKGLEWISYISTSSSTIYYADSVKGRFTISRDNAKNSLYLQMNSLRDEDTAVYYCAREGSYYDSSGYPYYYYDMDVWGQGTTVTVSSGGSEGKSSGSGSESKSTGGSQSVLTQPPSVSGAPGQRVTISCTGSSSNIGAGYDVHWYQQLPGTAPKLLIYGNTNRPSGVPDRFSGSKSGTSASLAITGLQAEDEADYYCQSYDSSLSGTPYVVFGGGTKLTVLGGGGSDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSLESRGPFEQKLISEEDLNMHTGHHHHHH

SEQ ID NO: 94 - DNA, 1686 bp, Artificial Sequence
  Description of Artificial Sequence Synthetic polynucleotide
atggagaccgacacactgctgctgtgggtgctgctgctgtgggtgcctggcagcacaggcgataccggagaggtgcagctggtggagtccggaggaggcctggtgcagccaggaggctctctgaggctgagctgcgcagcatccggcttcaccttttccttctacaacatgaattgggtgagacaggcaccaggcaagggcctggagtggatctcttatatcagcacaagctcctctaccatctactatgccgacagcgtgaagggccggtttacaatcagcagagataacgccaagaacagcctgtacctgcagatgaactctctgagggacgaggataccgccgtgtactattgtgcccgcgagggctcctactatgacagctccggctatccatactattactatgacatggacgtgtggggccagggcaccacagtgaccgtgtctagcggaggcagcgagggcaagtcctctggcagcggctccgagtctaagagcacaggaggctcccagtctgtgctgacccagccacctagcgtgtccggagcaccaggccagcgggtgacaatctcctgcaccggcagctcctctaatatcggcgccggctacgacgtgcactggtatcagcagctgcctggcacagccccaaagctgctgatctacggcaacaccaataggcccagcggcgtgcctgatcgcttttctggcagcaagtccggcacatctgccagcctggcaatcaccggactgcaggcagaggacgaggccgattactattgccagtcctacgacagctccctgtctggcaccccttatgtggtgttcggcggcggcacaaagctgaccgtgctgggaggaggaggcagcgatgcagtggtgacacaggagtccgccctgaccacatctccaggagagaccgtgacactgacctgtagatctagcaccggcgccgtgaccacatctaactacgccagctgggtgcaggagaagcctgaccacctgtttacaggcctgatcggaggcaccaacaatagggcaccaggcgtgcccgcaagattctccggctctctgatcggcgacaaggccgccctgacaatcaccggagcacagaccgaggatgaggccatctacttttgcgccctgtggtattccaatcactgggtctttggaggaggcacaaagctgaccgtgctgggtggaggaggaggcagcggcggaggaggctccggaggcggcggctctggcggcggcggcagcgacgtgcagctgcagcagagcggaccaggcctggtggcacccagccagtccctgtctatcacatgtaccgtgtccggcttctctctgaccgattacggcgtgaactgggtgcggcagtctcctggcaagggcctggagtggctgggcgtgatctggggcgacggcatcacagattataatagcgccctgaagagcaggctgtccgtgaccaaggataacagcaagtcccaggtgtttctgaagatgaacagcctgcagagcggcgactccgcccgctactattgcgtgacaggcctgttcgattactggggacagggcaccacactgaccgtgtcctctctcgagtctagagggcccttcgaacaaaaactcatctcagaagaggatctgaatatgcataccggtcatcatcaccatcaccattga

SEQ ID NO: 95 - protein, 542 aa, Artificial Sequence
  Description of Artificial Sequence Synthetic polypeptide
MAWVWTLLFLMAAAQSIQADIQMTQSPSSLSASVGDRVTITCRASQGIRNDLGWYQQKPGKAPKLLIYAASSLQSGVPSRFSGSGSGTDFTLTISSLQPEDFATYYCLQDYNYPLTFGGGTKVEIKGGSEGKSSGSGSESKSTGGSEVQLLESGGGLVQPGGSLRLSCAASGFTFSSYSMSWVRQAPGKGLEWVSVISGSGGFTDYADSVKGRFTISRDNSKNTLYLQMNSLRAEDTAVYYCARMPLNSPHDYWGQGTLVTVSSNSGGGGSDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSLESRGPFEQKLISEEDLNMHTGHHHHHH

SEQ ID NO: 96 - DNA, 1629 bp, Artificial Sequence
  Description of Artificial Sequence Synthetic polynucleotide
atggcctgggtgtggaccctgctgttcctgatggcagcagcacagtccatccaggccgacatccagatgacacagtctccaagctccctgagcgcctccgtgggcgacagggtgaccatcacatgcagggcaagccagggcatccggaacgatctgggctggtaccagcagaagcccggcaaggcccctaagctgctgatctatgcagcatctagcctgcagtccggagtgccatctcggttctctggcagcggctccggaaccgacttcaccctgacaatctcctctctgcagcctgaggacttcgccacatactattgcctgcaggattacaattatccactgacctttggcggcggcacaaaggtggagatcaagggaggctccgagggcaagagctccggctctggcagcgagtccaagtctaccggcggctctgaggtgcagctgctggagagcggaggaggactggtgcagccaggaggcagcctgcgcctgtcctgtgccgcctctggcttcaccttttctagctacagcatgtcctgggtgcggcaggcacctggcaagggactggagtgggtgagcgtgatctctggcagcggcggcttcacagactacgccgattccgtgaagggccggtttaccatcagcagagacaactccaagaatacactgtatctgcagatgaacagcctgagagccgaggacaccgccgtgtactattgtgccaggatgccactgaactctccccacgattattggggccagggcaccctggtgacagtgtcctctaattccggcggcggcggatccgatgcagtggtgacacaggagtccgccctgaccacatctccaggagagaccgtgacactgacctgtagatctagcaccggcgccgtgaccacatctaactacgccagctgggtgcaggagaagcctgaccacctgtttacaggcctgatcggaggcaccaacaatagggcaccaggcgtgcccgcaagattctccggctctctgatcggcgacaaggccgccctgacaatcaccggagcacagaccgaggatgaggccatctacttttgcgccctgtggtattccaatcactgggtctttggaggaggcacaaagctgaccgtgctgggtggaggaggaggcagcggcggaggaggctccggaggcggcggctctggcggcggcggcagcgacgtgcagctgcagcagagcggaccaggcctggtggcacccagccagtccctgtctatcacatgtaccgtgtccggcttctctctgaccgattacggcgtgaactgggtgcggcagtctcctggcaagggcctggagtggctgggcgtgatctggggcgacggcatcacagattataatagcgccctgaagagcaggctgtccgtgaccaaggataacagcaagtcccaggtgtttctgaagatgaacagcctgcagagcggcgactccgcccgctactattgcgtgacaggcctgttcgattactggggacagggcaccacactgaccgtgtcctctctcgagtctagagggcccttcgaacaaaaactcatctcagaagaggatctgaatatgcataccggtcatcatcaccatcaccattga

SEQ ID NO: 97 - protein, 542 aa, Artificial Sequence
  Description of Artificial Sequence Synthetic polypeptide
MAWVWTLLFLMAAAQSIQAQVQLQESGPGLVKPSETLSLTCTVSGVSISSYFWSWLRQPAGKGLQWIGRISTSGSTNHNPSLKSRVIMSVDTSKNQFSLKLSSVTAADTAVYYCVRDWTGFDYWGQGTLVTVSSGGSEGKSSGSGSESKSTGGSSYELTQPASVSGSPGQSITISCIGTSSDVGSYNLVSWYQQHPGKVPKLMIYEGSKRPSGVSNRFSGSKSGNTASLTISGLQAEDEADYYCSSYAGSSTYVFGTGTKVTVLNSGGGGSDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSLESRGPFEQKLISEEDLNMHTGHHHHHH

SEQ ID NO: 98 - DNA, 1629 bp, Artificial Sequence
  Description of Artificial Sequence Synthetic polynucleotide
atggcctgggtgtggaccctgctgttcctgatggcagcagcacagtccatccaggcacaggtgcagctgcaggagagcggaccaggactggtgaagccatctgagaccctgagcctgacctgcacagtgtctggcgtgagcatcagctcctacttttggagctggctgaggcagccagcaggcaagggactgcagtggatcggccgcatctccacctctggcagcacaaaccacaatccttccctgaagtctagagtgatcatgtccgtggacacctctaagaaccagttctccctgaagctgtctagcgtgaccgccgccgatacagccgtgtactattgcgtgagggactggacaggctttgattactggggccagggcaccctggtgacagtgtcctctggaggcagcgagggcaagagctccggctccggctctgagagcaagtccaccggcggctctagctatgagctgacacagcctgcatctgtgagcggctccccaggacagagcatcaccatctcctgtatcggcacatcctctgacgtgggctcctacaacctggtgtcttggtatcagcagcaccccggcaaggtgcctaagctgatgatctatgagggctccaagaggccaagcggcgtgtccaacagattctctggcagcaagtccggcaataccgcctctctgacaatcagcggactgcaggcagaggacgaggcagattactattgtagctcctacgccggctctagcacctacgtgttcggcaccggcacaaaggtgacagtgctgaatagcggcggcggcggatccgatgcagtggtgacacaggagtccgccctgaccacatctccaggagagaccgtgacactgacctgtagatctagcaccggcgccgtgaccacatctaactacgccagctgggtgcaggagaagcctgaccacctgtttacaggcctgatcggaggcaccaacaatagggcaccaggcgtgcccgcaagattctccggctctctgatcggcgacaaggccgccctgacaatcaccggagcacagaccgaggatgaggccatctacttttgcgccctgtggtattccaatcactgggtctttggaggaggcacaaagctgaccgtgctgggtggaggaggaggcagcggcggaggaggctccggaggcggcggctctggcggcggcggcagcgacgtgcagctgcagcagagcggaccaggcctggtggcacccagccagtccctgtctatcacatgtaccgtgtccggcttctctctgaccgattacggcgtgaactgggtgcggcagtctcctggcaagggcctggagtggctgggcgtgatctggggcgacggcatcacagattataatagcgccctgaagagcaggctgtccgtgaccaaggataacagcaagtcccaggtgtttctgaagatgaacagcctgcagagcggcgactccgcccgctactattgcgtgacaggcctgttcgattactggggacagggcaccacactgaccgtgtcctctctcgagtctagagggcccttcgaacaaaaactcatctcagaagaggatctgaatatgcataccggtcatcatcaccatcaccattga

SEQ ID NO: 99 - protein, 542 aa, Artificial Sequence
  Description of Artificial Sequence Synthetic polypeptide
MAWVWTLLFLMAAAQSIQASYELTQPASVSGSPGQSITISCIGTSSDVGSYNLVSWYQQHPGKVPKLMIYEGSKRPSGVSNRFSGSKSGNTASLTISGLQAEDEADYYCSSYAGSSTYVFGTGTKVTVLGGSEGKSSGSGSESKSTGGSQVQLQESGPGLVKPSETLSLTCTVSGVSISSYFWSWLRQPAGKGLQWIGRISTSGSTNHNPSLKSRVIMSVDTSKNQFSLKLSSVTAADTAVYYCVRDWTGFDYWGQGTLVTVSSNSGGGGSDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSLESRGPFEQKLISEEDLNMHTGHHHHHH

SEQ ID NO: 100 - DNA, 1629 bp, Artificial Sequence
  Description of Artificial Sequence Synthetic polynucleotide
atggcctgggtgtggaccctgctgttcctgatggcagcagcacagagcatccaggcctcctacgagctgacacagcctgcatctgtgagcggctccccaggacagtctatcaccatcagctgcatcggcacaagctccgacgtgggctcctacaacctggtgtcttggtatcagcagcaccccggcaaggtgcctaagctgatgatctatgagggcagcaagaggccaagcggcgtgtccaacagattctctggcagcaagtccggcaataccgcctccctgacaatctctggactgcaggcagaggacgaggcagattactattgctctagctacgccggctcctctacctacgtgttcggcaccggcacaaaggtgacagtgctgggaggctccgagggcaagagctccggctctggcagcgagtccaagtctaccggaggctcccaggtgcagctgcaggagagcggaccaggactggtgaagccaagcgagacactgtccctgacctgtacagtgtctggcgtgagcatctctagctacttttggagctggctgaggcagccagcaggcaagggactgcagtggatcggccgcatcagcacctccggctctacaaaccacaatccttccctgaagtctagagtgatcatgtctgtggacaccagcaagaaccagttctccctgaagctgtcctctgtgaccgccgccgatacagccgtgtactattgcgtgcgggactggaccggctttgattattggggccagggcaccctggtgacagtgagctccaatagcggcggcggcggatccgatgcagtggtgacacaggagtccgccctgaccacatctccaggagagaccgtgacactgacctgtagatctagcaccggcgccgtgaccacatctaactacgccagctgggtgcaggagaagcctgaccacctgtttacaggcctgatcggaggcaccaacaatagggcaccaggcgtgcccgcaagattctccggctctctgatcggcgacaaggccgccctgacaatcaccggagcacagaccgaggatgaggccatctacttttgcgccctgtggtattccaatcactgggtctttggaggaggcacaaagctgaccgtgctgggtggaggaggaggcagcggcggaggaggctccggaggcggcggctctggcggcggcggcagcgacgtgcagctgcagcagagcggaccaggcctggtggcacccagccagtccctgtctatcacatgtaccgtgtccggcttctctctgaccgattacggcgtgaactgggtgcggcagtctcctggcaagggcctggagtggctgggcgtgatctggggcgacggcatcacagattataatagcgccctgaagagcaggctgtccgtgaccaaggataacagcaagtcccaggtgtttctgaagatgaacagcctgcagagcggcgactccgcccgctactattgcgtgacaggcctgttcgattactggggacagggcaccacactgaccgtgtcctctctcgagtctagagggcccttcgaacaaaaactcatctcagaagaggatctgaatatgcataccggtcatcatcaccatcaccattga

SEQ ID NO: 101 - protein, 548 aa, Artificial Sequence
  Description of Artificial Sequence Synthetic polypeptide
MAWVWTLLFLMAAAQSIQAQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGGGSDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSGGGGSLESRGPFEQKLISEEDLNMHTGHHHHHH

SEQ ID NO: 102 - DNA, 1647 bp, Artificial Sequence
  Description of Artificial Sequence Synthetic polynucleotide
atggcttgggtttggaccctgctgttcctgatggccgctgctcagtctatccaggctcaggtgcagctgcaggagtccggaccaggcctggtgaagccaagccagaccctgtccctgacctgcacagtgtccggcaactctatcacaagcgactatgcctggaattggatcaggcagttccctggcaagcgcctggagtggatcggctatatctcttacagcggctccaccacatacaacccctccctgaagtctcgggtgaccatcagccgggacacaagcaagaatcagttcagcctgaagctgagctccgtgaccgcagcagatacagccgtgtactattgcgccaccggctactattacggctccggattttggggacagggcaccctggtgacagtgtctagcggaggcagcgagggcaagtcctctggctctggcagcgagtccaagtctaccggcggcagcgagatcgtgctgacccagtcccctgccacactgagcctgtccccaggagagagggccaccctgtcttgtagagcctctgagagcgtggagtatttcggcacaagcctgatgcactggtatcagcagaagccaggccagccccctaggctgctgatctatgccgcctccaacgtggagtctggcatccccgcacgcttctccggctctggcagcggcaccgactttaccctgacaatcagctccgtggagcccgaggatttcgccgtgtatttttgtcagcagacacggaaggtgccttacacctttggcggcggcacaaaggtggagatcaagggaggaggaggatccgacgcagtggtgacacaggagtctgccctgaccacaagcccaggcgagaccgtgacactgacctgtaggtcctctaccggcgccgtgaccacatccaattacgcctcttgggtgcaggagaagcccgatcacctgttcacaggcctgatcggaggcaccaacaatagggcaccaggcgtgcccgccagattttctggcagcctgatcggcgacaaggccgccctgacaatcaccggagcacagaccgaggatgaggccatctacttctgcgccctgtggtatagcaaccactgggtgtttggcggcggcacaaagctgaccgtgctgggaggaggaggaggctccggcggaggaggctctggcggcggcggcagcggaggcggcggctccgacgtgcagctgcagcagtccggacctggcctggtggcaccatcccagtctctgagcatcacatgtaccgtgagcggcttttccctgaccgattacggagtgaactgggtgcggcagagcccaggcaagggactggagtggctgggcgtgatctggggcgacggcatcacagattataattccgccctgaagtctaggctgagcgtgaccaaggataactccaagtctcaggtgttcctgaagatgaacagcctgcagtctggcgacagcgcccgctactattgcgtgacaggcctgtttgattactggggccagggcaccacactgaccgtgagctccggcggcggcggcagcctcgagtctagagggcccttcgaacaaaaactcatctcagaagaggatctgaatatgcataccggtcatcatcaccatcaccattga

SEQ ID NO: 103 - protein, 548 aa, Artificial Sequence
  Description of Artificial Sequence Synthetic polypeptide
MAWVWTLLFLMAAAQSIQAEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSGGGGSDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSGGGGSLESRGPFEQKLISEEDLNMHTGHHHHHH

SEQ ID NO: 104 - DNA, 1647 bp, Artificial Sequence
  Description of Artificial Sequence Synthetic polynucleotide
atggcttgggtttggaccctgctgttcctgatggccgctgctcagtctatccaggctgagatcgtgctgacccagtcccctgccacactgtctctgagcccaggagagagggccaccctgtcttgcagggcatccgagtctgtggagtatttcggcacaagcctgatgcactggtatcagcagaagccaggccagccccctaggctgctgatctatgcagcaagcaacgtggagtccggcatccccgcacgcttcagcggctccggctctggcaccgactttaccctgacaatcagctccgtggagcccgaggatttcgccgtgtatttttgccagcagacacggaaggtgccttacacctttggcggcggcacaaaggtggagatcaagggaggctccgagggcaagtctagcggcagcggctccgagtctaagagcaccggaggcagccaggtgcagctgcaggagtccggaccaggcctggtgaagccatctcagaccctgagcctgacctgtacagtgtccggcaactctatcacaagcgactatgcctggaattggatcagacagttccctggcaagagactggagtggatcggctatatctcctactctggcagcaccacatacaacccctccctgaagtctcgggtgaccatctccagagacacatctaagaatcagttcagcctgaagctgtcctctgtgaccgccgccgatacagccgtgtactattgtgccaccggctactattacggctccggattttggggacagggcaccctggtgacagtgagctccggaggaggaggatccgacgcagtggtgacacaggagtctgccctgaccacaagcccaggcgagaccgtgacactgacctgtaggtcctctaccggcgccgtgaccacatccaattacgcctcttgggtgcaggagaagcccgatcacctgttcacaggcctgatcggaggcaccaacaatagggcaccaggcgtgcccgccagattttctggcagcctgatcggcgacaaggccgccctgacaatcaccggagcacagaccgaggatgaggccatctacttctgcgccctgtggtatagcaaccactgggtgtttggcggcggcacaaagctgaccgtgctgggaggaggaggaggctccggcggaggaggctctggcggcggcggcagcggaggcggcggctccgacgtgcagctgcagcagtccggacctggcctggtggcaccatcccagtctctgagcatcacatgtaccgtgagcggcttttccctgaccgattacggagtgaactgggtgcggcagagcccaggcaagggactggagtggctgggcgtgatctggggcgacggcatcacagattataattccgccctgaagtctaggctgagcgtgaccaaggataactccaagtctcaggtgttcctgaagatgaacagcctgcagtctggcgacagcgcccgctactattgcgtgacaggcctgtttgattactggggccagggcaccacactgaccgtgagctccggcggcggcggcagcctcgagtctagagggcccttcgaacaaaaactcatctcagaagaggatctgaatatgcataccggtcatcatcaccatcaccattga

SEQ ID NO: 105 - protein, 543 aa, Artificial Sequence
  Description of Artificial Sequence Synthetic polypeptide
MAWVWTLLFLMAAAQSIQADAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSGGGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSGGSEGKSSGSGSESKSTGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKLESRGPFEQKLISEEDLNMHTGHHHHHH

SEQ ID NO: 106 - DNA, 1632 bp, Artificial Sequence
  Description of Artificial Sequence Synthetic polynucleotide
atggcttgggtttggaccctgctgttcctgatggccgctgctcagtctatccaggctgatgcagtggtgacacaggagagcgccctgaccacatccccaggagagaccgtgacactgacctgcaggagctccaccggagcagtgaccacatccaactacgcctcttgggtgcaggagaagcccgatcacctgttcacaggcctgatcggcggcaccaacaatagggcaccaggcgtgcccgcacgcttttctggcagcctgatcggcgacaaggccgccctgacaatcaccggagcacagacagaggatgaggccatctacttctgcgccctgtggtatagcaatcactgggtgtttggcggcggcacaaagctgaccgtgctgggaggaggaggaggctctggaggaggaggcagcggcggcggcggctccggcggcggcggctctgacgtgcagctgcagcagtccggacctggcctggtggcaccatcccagtctctgagcatcacatgtaccgtgagcggcttctccctgaccgattacggagtgaactgggtgcggcagtcccctggcaagggactggagtggctgggcgtgatctggggcgacggcatcacagattataattctgccctgaagtctaggctgagcgtgaccaaggacaactccaagtctcaggtgttcctgaagatgaacagcctgcagtctggcgacagcgcccgctactattgcgtgacaggcctgtttgattactggggccagggcaccacactgaccgtgtcttcgggaggaggaggatcccaggtgcagctgcaggagtccggaccaggcctggtgaagccaagccagaccctgtccctgacctgcacagtgtccggcaactctatcacaagcgactatgcctggaattggatcaggcagttccctggcaagcgcctggagtggatcggctatatctcttacagcggctccaccacatacaacccctccctgaagtctcgggtgaccatcagccgggacacaagcaagaatcagttcagcctgaagctgagctccgtgaccgcagcagatacagccgtgtactattgcgccaccggctactattacggctccggattttggggacagggcaccctggtgacagtgtctagcggaggcagcgagggcaagtcctctggctctggcagcgagtccaagtctaccggcggcagcgagatcgtgctgacccagtcccctgccacactgagcctgtccccaggagagagggccaccctgtcttgtagagcctctgagagcgtggagtatttcggcacaagcctgatgcactggtatcagcagaagccaggccagccccctaggctgctgatctatgccgcctccaacgtggagtctggcatccccgcacgcttctccggctctggcagcggcaccgactttaccctgacaatcagctccgtggagcccgaggatttcgccgtgtatttttgtcagcagacacggaaggtgccttacacctttggcggcggcacaaaggtggagatcaagctcgagtctagagggcccttcgaacaaaaactcatctcagaagaggatctgaatatgcataccggtcatcatcaccatcaccattga

SEQ ID NO: 107 - protein, 543 aa, Artificial Sequence
  Description of Artificial Sequence Synthetic polypeptide
MAWVWTLLFLMAAAQSIQADAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSGGGGSEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSLESRGPFEQKLISEEDLNMHTGHHHHHH

SEQ ID NO: 108 - DNA, 1632 bp, Artificial Sequence
  Description of Artificial Sequence Synthetic polynucleotide
atggcttgggtttggaccctgctgttcctgatggccgctgctcagtctatccaggctgatgcagtggtgacacaggagagcgccctgaccacatccccaggagagaccgtgacactgacctgcaggagctccaccggagcagtgaccacatccaactacgcctcttgggtgcaggagaagcccgatcacctgttcacaggcctgatcggcggcaccaacaatagggcaccaggcgtgcccgcacgcttttctggcagcctgatcggcgacaaggccgccctgacaatcaccggagcacagacagaggatgaggccatctacttctgcgccctgtggtatagcaatcactgggtgtttggcggcggcacaaagctgaccgtgctgggaggaggaggaggctctggaggaggaggcagcggcggcggcggctccggcggcggcggctctgacgtgcagctgcagcagtccggacctggcctggtggcaccatcccagtctctgagcatcacatgtaccgtgagcggcttctccctgaccgattacggagtgaactgggtgcggcagtcccctggcaagggactggagtggctgggcgtgatctggggcgacggcatcacagattataattctgccctgaagtctaggctgagcgtgaccaaggacaactccaagtctcaggtgttcctgaagatgaacagcctgcagtctggcgacagcgcccgctactattgcgtgacaggcctgtttgattactggggccagggcaccacactgaccgtgtcttcgggaggaggaggatccgagatcgtgctgacccagtcccctgccacactgtctctgagcccaggagagagggccaccctgtcttgcagggcatccgagtctgtggagtatttcggcacaagcctgatgcactggtatcagcagaagccaggccagccccctaggctgctgatctatgcagcaagcaacgtggagtccggcatccccgcacgcttcagcggctccggctctggcaccgactttaccctgacaatcagctccgtggagcccgaggatttcgccgtgtatttttgccagcagacacggaaggtgccttacacctttggcggcggcacaaaggtggagatcaagggaggctccgagggcaagtctagcggcagcggctccgagtctaagagcaccggaggcagccaggtgcagctgcaggagtccggaccaggcctggtgaagccatctcagaccctgagcctgacctgtacagtgtccggcaactctatcacaagcgactatgcctggaattggatcagacagttccctggcaagagactggagtggatcggctatatctcctactctggcagcaccacatacaacccctccctgaagtctcgggtgaccatctccagagacacatctaagaatcagttcagcctgaagctgtcctctgtgaccgccgccgatacagccgtgtactattgtgccaccggctactattacggctccggattttggggacagggcaccctggtgacagtgagctccctcgagtctagagggcccttcgaacaaaaactcatctcagaagaggatctgaatatgcataccggtcatcatcaccatcaccattga

SEQ ID NO: 109 - protein, 464 aa, Artificial Sequence
  Description of Artificial Sequence Synthetic polypeptide
METDTLLLWVLLLWVPGSTGDGSDLGKKLLEAARAGQDDEVRILMANGADVNASDRYGRTPLHLAAFNGHLEIVEVLLKNGADVNAKDKIGNTPLHLAANHGHLEIVEVLLKYGAVVNATDWLGVTPLHLAAVFGHLEIVEVLLKYGADVNAQDKFGKTAFDISIDNGNEDLAEILQKLNSGGGGSTGDAVVTQESALTTSPGETVTLTCRSSTGAVTTSNYASWVQEKPDHLFTGLIGGTNNRAPGVPARFSGSLIGDKAALTITGAQTEDEAIYFCALWYSNHWVFGGGTKLTVLGGGGGSGGGGSGGGGSGGGGSDVQLQQSGPGLVAPSQSLSITCTVSGFSLTDYGVNWVRQSPGKGLEWLGVIWGDGITDYNSALKSRLSVTKDNSKSQVFLKMNSLQSGDSARYYCVTGLFDYWGQGTTLTVSSGGGGSLESRGPFEQKLISEEDLNMHTGHHHHHH

SEQ ID NO: 110 - DNA, 1395 bp, Artificial Sequence
  Description of Artificial Sequence Synthetic polynucleotide
atggagaccgacacactgctgctgtgggtgctgctgctgtgggtgcccggctctaccggcgacggcagcgatctgggcaagaagctgctggaggcagccagagccggacaggacgatgaggtgagaatcctgatggccaacggcgccgacgtgaatgccagcgatcggtacggcagaacaccactgcacctggcagccttcaacggacacctggagatcgtggaggtgctgctgaagaatggagccgacgtgaatgccaaggataagatcggcaacacccctctgcacctggcagcaaatcatggccacctggagattgtcgaggtgctgctgaagtacggcgccgtggtgaatgccacagactggctgggagtgacccccctgcacctggccgccgtgtttggccacctggagatcgtcgaagtcctgctgaagtatggcgccgacgtgaacgcccaggataagttcggcaagacagcctttgacatctccatcgataacggcaatgaggacctggccgagatcctgcagaagctgaattctggaggaggaggctctacaggcgatgcagtggtgacccaggagagcgccctgaccacatcccctggagagaccgtgacactgacctgccggagctccaccggagcagtgaccacaagcaactatgcctcctgggtgcaggagaagccagatcacctgttcacaggcctgatcggaggcaccaacaatagggcaccaggcgtgcctgcacgcttttccggctctctgatcggcgacaaggccgccctgacaatcaccggagcacagaccgaggatgaggccatctacttctgcgccctgtggtattctaatcactgggtgtttggcggcggcacaaagctgaccgtgctgggaggaggaggaggctctggaggaggaggcagcggcggcggcggctccggcggcggcggctctgacgtgcagctgcagcagtccggaccaggcctggtggcacccagccagtccctgtctatcacatgtaccgtgtctggcttcagcctgaccgattacggagtgaactgggtgcggcagagccctggcaagggactggagtggctgggcgtgatctggggcgacggcatcacagattataattccgccctgaagtccaggctgtctgtgaccaaggataacagcaagtcccaggtgttcctgaagatgaatagcctgcagtccggcgactctgcccgctactattgcgtgacaggcctgtttgattactggggccagggcaccacactgaccgtgtctagcggcggcggcggcagcctcgagtctagagggcccttcgaacaaaaactcatctcagaagaggatctgaatatgcataccggtcatcatcaccatcaccattga

SEQ ID NO: 111 - protein, 373 aa, Artificial Sequence
  Description of Artificial Sequence Synthetic polypeptide
METDTLLLWVLLLWVPGSTGQSVLTQPPSVSGAPGQRVTISCTGSSSNIGAGYDVHWYQQLPGTAPKLLIYGNTNRPSGVPDRFSGSKSGTSASLAITGLQAEDEADYYCQSYDSSLSGTPYVVFGGGTKLTVLGGSEGKSSGSGSESKSTGGSEVQLVESGGGLVQPGGSLRLSCAASGFTFSFYNMNWVRQAPGKGLEWISYISTSSSTIYYADSVKGRFTISRDNAKNSLYLQMNSLRDEDTAVYYCAREGSYYDSSGYPYYYYDMDVWGQGTTVTVSSGGGGSGGGGSGGGGSLEIEAAFLERENTALETRVAELRQRVQRLRNRVSQYRTRYGPLGGGGSLESRGPFEQKLISEEDLNMHTGHHHHHH

SEQ ID NO: 112 - DNA, 1122 bp, Artificial Sequence
  Description of Artificial Sequence Synthetic polynucleotide
atggagacagacacactgctgctgtgggtgctgctgctgtgggtaccaggcagcacaggccagtctgtgctgacccagccacctagcgtgtccggagcaccaggccagcgggtgacaatctcctgcaccggcagctcctctaacatcggcgccggctacgacgtgcactggtatcagcagctgcctggcacagccccaaagctgctgatctacggcaacaccaataggcccagcggcgtgcctgatcgcttttctggcagcaagtccggcacatctgccagcctggcaatcaccggactgcaggcagaggacgaggccgattactattgccagtcttacgacagctccctgagcggcacaccttatgtggtgttcggaggaggcacaaagctgaccgtgctgggaggcagcgagggcaagtctagcggctccggctctgagagcaagtccaccggaggcagcgaggtgcagctggtggagtccggaggaggcctggtgcagccaggaggcagcctgcggctgtcctgtgccgcctctggcttcaccttttccttctacaacatgaattgggtgagacaggcacctggcaagggcctggagtggatcagctatatctccacatcctctagcaccatctactatgccgacagcgtgaagggccggtttacaatcagccgggacaacgccaagaatagcctgtacctgcagatgaacagcctgagggacgaggataccgccgtgtactattgcgcccgcgagggctcctactatgactcctctggctatccatactattactatgacatggacgtgtggggccagggcaccacagtgacagtgagctccggaggaggaggatccggcggcggaggatctggcggcggaggcagcctggaaatcgaggccgccttcctggaacgggaaaacaccgccctggagacaagagtcgccgagctgagacagcgggtgcagagactgcggaatagagtgtcccaataccgcaccagatacggccctctgggcggcggcggcagcctcgagtctagagggcccttcgaacaaaaactcatctcagaagaggatctgaatatgcataccggtcatcatcaccatcaccattga

SEQ ID NO: 113 - protein, 358 aa, Artificial Sequence
  Description of Artificial Sequence Synthetic polypeptide
MAWVWTLLFLMAAAQSIQAEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSGGGGSGGGGSGGGGSLEIEAAFLERENTALETRVAELRQRVQRLRNRVSQYRTRYGPLGGGGSLESRGPFEQKLISEEDLNMHTGHHHHHH

SEQ ID NO: 114 - DNA, 1077 bp, Artificial Sequence
  Description of Artificial Sequence Synthetic polynucleotide
atggcttgggtttggaccctgctgttcctgatggccgctgctcagtctatccaggctgagatcgtgctgacccagtcccctgccacactgtctctgagcccaggagagagggccaccctgtcttgcagggcatccgagtctgtggagtatttcggcacaagcctgatgcactggtatcagcagaagccaggccagccccctaggctgctgatctatgcagcaagcaacgtggagtccggcatccccgcacgcttcagcggctccggctctggcaccgactttaccctgacaatcagctccgtggagcccgaggatttcgccgtgtatttttgccagcagacacggaaggtgccttacacctttggcggcggcacaaaggtggagatcaagggaggctccgagggcaagtctagcggcagcggctccgagtctaagagcaccggaggcagccaggtgcagctgcaggagtccggaccaggcctggtgaagccatctcagaccctgagcctgacctgtacagtgtccggcaactctatcacaagcgactatgcctggaattggatcagacagttccctggcaagagactggagtggatcggctatatctcctactctggcagcaccacatacaacccctccctgaagtctcgggtgaccatctccagagacacatctaagaatcagttcagcctgaagctgtcctctgtgaccgccgccgatacagccgtgtactattgtgccaccggctactattacggctccggattttggggacagggcaccctggtgacagtgagctccggaggaggaggatccggcggcggaggatctggcggcggaggcagcctggaaatcgaggccgccttcctggaacgggaaaacaccgccctggagacaagagtcgccgagctgagacagcgggtgcagagactgcggaatagagtgtcccaataccgcaccagatacggccctctgggcggcggcggcagcctcgagtctagagggcccttcgaacaaaaactcatctcagaagaggatctgaatatgcataccggtcatcatcaccatcaccattga

SEQ ID NO: 115 - protein, 357 aa, Artificial Sequence
  Description of Artificial Sequence Synthetic polypeptide
MAWVWTLLFLMAAAQSIQASYELTQPASVSGSPGQSITISCIGTSSDVGSYNLVSWYQQHPGKVPKLMIYEGSKRPSGVSNRFSGSKSGNTASLTISGLQAEDEADYYCSSYAGSSTYVFGTGTKVTVLGGSEGKSSGSGSESKSTGGSQVQLQESGPGLVKPSETLSLTCTVSGVSISSYFWSWLRQPAGKGLQWIGRISTSGSTNHNPSLKSRVIMSVDTSKNQFSLKLSSVTAADTAVYYCVRDWTGFDYWGQGTLVTVSSNSGGGGSGGGGSGGGGSLEIEAAFLERENTALETRVAELRQRVQRLRNRVSQYRTRYGPLGGGGSLESRGPFEQKLISEEDLNMHTGHHHHHH

SEQ ID NO: 116 - DNA, 1074 bp, Artificial Sequence
  Description of Artificial Sequence Synthetic polynucleotide
atggcctgggtgtggaccctgctgttcctgatggcagcagcacagagcatccaggcctcctacgagctgacacagcctgcatctgtgagcggctccccaggacagtctatcaccatcagctgcatcggcacaagctccgacgtgggctcctacaacctggtgtcttggtatcagcagcaccccggcaaggtgcctaagctgatgatctatgagggcagcaagaggccaagcggcgtgtccaacagattctctggcagcaagtccggcaataccgcctccctgacaatctctggactgcaggcagaggacgaggcagattactattgctctagctacgccggctcctctacctacgtgttcggcaccggcacaaaggtgacagtgctgggaggctccgagggcaagagctccggctctggcagcgagtccaagtctaccggaggctcccaggtgcagctgcaggagagcggaccaggactggtgaagccaagcgagacactgtccctgacctgtacagtgtctggcgtgagcatctctagctacttttggagctggctgaggcagccagcaggcaagggactgcagtggatcggccgcatcagcacctccggctctacaaaccacaatccttccctgaagtctagagtgatcatgtctgtggacaccagcaagaaccagttctccctgaagctgtcctctgtgaccgccgccgatacagccgtgtactattgcgtgcgggactggaccggctttgattattggggccagggcaccctggtgacagtgagctccaatagcggcggcggcggatccggcggcggaggatctggcggcggaggcagcctggaaatcgaggccgccttcctggaacgggaaaacaccgccctggagacaagagtcgccgagctgagacagcgggtgcagagactgcggaatagagtgtcccaataccgcaccagatacggccctctgggcggcggcggcagcctcgagtctagagggcccttcgaacaaaaactcatctcagaagaggatctgaatatgcataccggtcatcatcaccatcaccattga

SEQ ID NO: 117 - protein, 567 aa, Artificial Sequence
  Description of Artificial Sequence Synthetic polypeptide
METDTLLLWVLLLWVPGSTGQSVLTQPPSVSGAPGQRVTISCTGSSSNIGAGYDVHWYQQLPGTAPKLLIYGNTNRPSGVPDRFSGSKSGTSASLAITGLQAEDEADYYCQSYDSSLSGTPYVVFGGGTKLTVLGGSEGKSSGSGSESKSTGGSEVQLVESGGGLVQPGGSLRLSCAASGFTFSFYNMNWVRQAPGKGLEWISYISTSSSTIYYADSVKGRFTISRDNAKNSLYLQMNSLRDEDTAVYYCAREGSYYDSSGYPYYYYDMDVWGQGTTVTVSSGGGGSQVQLVQSGAEVKKPGASVKVSCKASGYTFTHYYIYWVRQAPGQGLEWMGGVNPSNGGTHFNEKFKSRVTMTRDTSISTAYMELSRLRSDDTAVYYCARSEYDYGLGFAYWGQGTLVTVSSGGSEGKSSGSGSESKSTGGSDIVMTQSPDSLAVSLGERATINCKSSQSLLNSRTPKNYLAWYQQKPGQPPKLLIYWASTRKSGVPDRFSGSGSGTDFTLTISSLQAEDVAVYYCKQSYNLLTFGGGTKVEIKLESRGPFEQKLISEEDLNMHTGHHHHHH

SEQ ID NO: 118 - DNA, 1704 bp, Artificial Sequence
  Description of Artificial Sequence Synthetic polynucleotide
atggagacagacacactgctgctgtgggtgctgctgctgtgggtaccaggcagcacaggccagtctgtgctgacccagccacctagcgtgtccggagcaccaggccagcgggtgacaatctcctgcaccggcagctcctctaacatcggcgccggctacgacgtgcactggtatcagcagctgcctggcacagccccaaagctgctgatctacggcaacaccaataggcccagcggcgtgcctgatcgcttttctggcagcaagtccggcacatctgccagcctggcaatcaccggactgcaggcagaggacgaggccgattactattgccagtcttacgacagctccctgagcggcacaccttatgtggtgttcggaggaggcacaaagctgaccgtgctgggaggcagcgagggcaagtctagcggctccggctctgagagcaagtccaccggaggcagcgaggtgcagctggtggagtccggaggaggcctggtgcagccaggaggcagcctgcggctgtcctgtgccgcctctggcttcaccttttccttctacaacatgaattgggtgagacaggcacctggcaagggcctggagtggatcagctatatctccacatcctctagcaccatctactatgccgacagcgtgaagggccggtttacaatcagccgggacaacgccaagaatagcctgtacctgcagatgaacagcctgagggacgaggataccgccgtgtactattgcgcccgcgagggctcctactatgactcctctggctatccatactattactatgacatggacgtgtggggccagggcaccacagtgacagtgagctccggaggaggaggatcccaagtgcaactggtccagtcaggtgctgaggtgaaaaaacccggagccagtgtcaaagtaagctgcaaggcctctgggtatactttcacccattactatatatactgggttcgtcaagctccaggtcaggggcttgagtggatgggtggagtcaacccttcgaacggtggcactcacttcaatgaaaagtttaaaagccgcgtaaccatgacgcgagatacttccatttccacagcttatatggaacttagtaggttacgcagtgatgacacggccgtttattactgtgctagaagtgaatatgattatgggttgggtttcgcttactggggccagggaaccctcgtcaccgtgtccagtggaggcagcgagggcaagtctagcggctccggctctgagagcaagtccaccggaggcagcgacattgttatgacgcagagccctgattcactcgcagtgtccctaggagagcgggccaccatcaactgtaaaagttctcagtccctgctgaacagcaggacgcctaagaattacctggcatggtaccaacagaaacctggacagccgcctaagctgctcatttactgggcctccacacggaagagcggcgtgcccgaccggttttccgggagcggctccggcaccgactttaccttgaccatcagttccctgcaggcagaagacgtggccgtatactattgcaagcaatcttacaatctcctgacatttggcggcggcacaaaagtggagatcaaactcgagtctagagggcccttcgaacaaaaactcatctcagaagaggatctgaatatgcataccggtcatcatcaccatcaccattga

SEQ ID NO: 119 - protein, 552 aa, Artificial Sequence
  Description of Artificial Sequence Synthetic polypeptide
MAWVWTLLFLMAAAQSIQAEIVLTQSPATLSLSPGERATLSCRASESVEYFGTSLMHWYQQKPGQPPRLLIYAASNVESGIPARFSGSGSGTDFTLTISSVEPEDFAVYFCQQTRKVPYTFGGGTKVEIKGGSEGKSSGSGSESKSTGGSQVQLQESGPGLVKPSQTLSLTCTVSGNSITSDYAWNWIRQFPGKRLEWIGYISYSGSTTYNPSLKSRVTISRDTSKNQFSLKLSSVTAADTAVYYCATGYYYGSGFWGQGTLVTVSSGGGGSQVQLVQSGAEVKKPGASVKVSCKASGYTFTHYYIYWVRQAPGQGLEWMGGVNPSNGGTHFNEKFKSRVTMTRDTSISTAYMELSRLRSDDTAVYYCARSEYDYGLGFAYWGQGTLVTVSSGGSEGKSSGSGSESKSTGGSDIVMTQSPDSLAVSLGERATINCKSSQSLLNSRTPKNYLAWYQQKPGQPPKLLIYWASTRKSGVPDRFSGSGSGTDFTLTISSLQAEDVAVYYCKQSYNLLTFGGGTKVEIKLESRGPFEQKLISEEDLNMHTGHHHHHH

SEQ ID NO: 120 - DNA, 1659 bp, Artificial Sequence
  Description of Artificial Sequence Synthetic polynucleotide
atggcttgggtttggaccctgctgttcctgatggccgctgctcagtctatccaggctgagatcgtgctgacccagtcccctgccacactgtctctgagcccaggagagagggccaccctgtcttgcagggcatccgagtctgtggagtatttcggcacaagcctgatgcactggtatcagcagaagccaggccagccccctaggctgctgatctatgcagcaagcaacgtggagtccggcatccccgcacgcttcagcggctccggctctggcaccgactttaccctgacaatcagctccgtggagcccgaggatttcgccgtgtatttttgccagcagacacggaaggtgccttacacctttggcggcggcacaaaggtggagatcaagggaggctccgagggcaagtctagcggcagcggctccgagtctaagagcaccggaggcagccaggtgcagctgcaggagtccggaccaggcctggtgaagccatctcagaccctgagcctgacctgtacagtgtccggcaactctatcacaagcgactatgcctggaattggatcagacagttccctggcaagagactggagtggatcggctatatctcctactctggcagcaccacatacaacccctccctgaagtctcgggtgaccatctccagagacacatctaagaatcagttcagcctgaagctgtcctctgtgaccgccgccgatacagccgtgtactattgtgccaccggctactattacggctccggattttggggacagggcaccctggtgacagtgagctccggaggaggaggatcccaagtgcaactggtccagtcaggtgctgaggtgaaaaaacccggagccagtgtcaaagtaagctgcaaggcctctgggtatactttcacccattactatatatactgggttcgtcaagctccaggtcaggggcttgagtggatgggtggagtcaacccttcgaacggtggcactcacttcaatgaaaagtttaaaagccgcgtaaccatgacgcgagatacttccatttccacagcttatatggaacttagtaggttacgcagtgatgacacggccgtttattactgtgctagaagtgaatatgattatgggttgggtttcgcttactggggccagggaaccctcgtcaccgtgtccagtggaggcagcgagggcaagtctagcggctccggctctgagagcaagtccaccggaggcagcgacattgttatgacgcagagccctgattcactcgcagtgtccctaggagagcgggccaccatcaactgtaaaagttctcagtccctgctgaacagcaggacgcctaagaattacctggcatggtaccaacagaaacctggacagccgcctaagctgctcatttactgggcctccacacggaagagcggcgtgcccgaccggttttccgggagcggctccggcaccgactttaccttgaccatcagttccctgcaggcagaagacgtggccgtatactattgcaagcaatcttacaatctcctgacatttggcggcggcacaaaagtggagatcaaactcgagtctagagggcccttcgaacaaaaactcatctcagaagaggatctgaatatgcataccggtcatcatcaccatcaccattga

SEQ ID NO: 121 - protein, 551 aa, Artificial Sequence
  Description of Artificial Sequence Synthetic polypeptide
MAWVWTLLFLMAAAQSIQASYELTQPASVSGSPGQSITISCIGTSSDVGSYNLVSWYQQHPGKVPKLMIYEGSKRPSGVSNRFSGSKSGNTASLTISGLQAEDEADYYCSSYAGSSTYVFGTGTKVTVLGGSEGKSSGSGSESKSTGGSQVQLQESGPGLVKPSETLSLTCTVSGVSISSYFWSWLRQPAGKGLQWIGRISTSGSTNHNPSLKSRVIMSVDTSKNQFSLKLSSVTAADTAVYYCVRDWTGFDYWGQGTLVTVSSNSGGGGSQVQLVQSGAEVKKPGASVKVSCKASGYTFTHYYIYWVRQAPGQGLEWMGGVNPSNGGTHFNEKFKSRVTMTRDTSISTAYMELSRLRSDDTAVYYCARSEYDYGLGFAYWGQGTLVTVSSGGSEGKSSGSGSESKSTGGSDIVMTQSPDSLAVSLGERATINCKSSQSLLNSRTPKNYLAWYQQKPGQPPKLLIYWASTRKSGVPDRFSGSGSGTDFTLTISSLQAEDVAVYYCKQSYNLLTFGGGTKVEIKLESRGPFEQKLISEEDLNMHTGHHHHHH

SEQ ID NO: 122 - DNA, 1656 bp, Artificial Sequence
  Description of Artificial Sequence Synthetic polynucleotide
atggcctgggtgtggaccctgctgttcctgatggcagcagcacagagcatccaggcctcctacgagctgacacagcctgcatctgtgagcggctccccaggacagtctatcaccatcagctgcatcggcacaagctccgacgtgggctcctacaacctggtgtcttggtatcagcagcaccccggcaaggtgcctaagctgatgatctatgagggcagcaagaggccaagcggcgtgtccaacagattctctggcagcaagtccggcaataccgcctccctgacaatctctggactgcaggcagaggacgaggcagattactattgctctagctacgccggctcctctacctacgtgttcggcaccggcacaaaggtgacagtgctgggaggctccgagggcaagagctccggctctggcagcgagtccaagtctaccggaggctcccaggtgcagctgcaggagagcggaccaggactggtgaagccaagcgagacactgtccctgacctgtacagtgtctggcgtgagcatctctagctacttttggagctggctgaggcagccagcaggcaagggactgcagtggatcggccgcatcagcacctccggctctacaaaccacaatccttccctgaagtctagagtgatcatgtctgtggacaccagcaagaaccagttctccctgaagctgtcctctgtgaccgccgccgatacagccgtgtactattgcgtgcgggactggaccggctttgattattggggccagggcaccctggtgacagtgagctccaatagcggcggcggcggatcccaagtgcaactggtccagtcaggtgctgaggtgaaaaaacccggagccagtgtcaaagtaagctgcaaggcctctgggtatactttcacccattactatatatactgggttcgtcaagctccaggtcaggggcttgagtggatgggtggagtcaacccttcgaacggtggcactcacttcaatgaaaagtttaaaagccgcgtaaccatgacgcgagatacttccatttccacagcttatatggaacttagtaggttacgcagtgatgacacggccgtttattactgtgctagaagtgaatatgattatgggttgggtttcgcttactggggccagggaaccctcgtcaccgtgtccagtggaggcagcgagggcaagtctagcggctccggctctgagagcaagtccaccggaggcagcgacattgttatgacgcagagccctgattcactcgcagtgtccctaggagagcgggccaccatcaactgtaaaagttctcagtccctgctgaacagcaggacgcctaagaattacctggcatggtaccaacagaaacctggacagccgcctaagctgctcatttactgggcctccacacggaagagcggcgtgcccgaccggttttccgggagcggctccggcaccgactttaccttgaccatcagttccctgcaggcagaagacgtggccgtatactattgcaagcaatcttacaatctcctgacatttggcggcggcacaaaagtggagatcaaactcgagtctagagggcccttcgaacaaaaactcatctcagaagaggatctgaatatgcataccggtcatcatcaccatcaccattga

SEQ ID NO: 123 - protein, 7 aa, Artificial Sequence
  Description of Artificial Sequence Synthetic peptide
NSGGGGS

SEQ ID NO: 124 - protein, 5 aa, Artificial Sequence
  Description of Artificial Sequence Synthetic peptide
GGGGS

SEQ ID NO: 125 - protein, 9 aa, Artificial Sequence
  Description of Artificial Sequence Synthetic peptide
NSGGGGSTG

SEQ ID NO: 126 - protein, 15 aa, Artificial Sequence
  Description of Artificial Sequence Synthetic peptide
GGGGSGGGGSGGGGS

SEQ ID NO: 127 - protein, 17 aa, Artificial Sequence
  Description of Artificial Sequence Synthetic peptide
NSGGGGSGGGGSGGGGS

SEQ ID NO: 128 - protein, 10 aa, Artificial Sequence
  Description of Artificial Sequence Synthetic peptide
  See specification as filed for detailed description of substitutions and preferred embodiments
GGGGSGGGGS

SEQ ID NO: 129 - protein, 36 aa, Artificial Sequence
  Description of Artificial Sequence Synthetic polypeptide
SGLEQLESIINFEKLTEWTSHMGSSYSLESIGTSHM

SEQ ID NO: 130 - DNA, 34 bp, Artificial Sequence
  Description of Artificial Sequence Synthetic oligonucleotide
gaagttcctattctctagaaagtataggaacttc

SEQ ID NO: 131 - protein, 21 aa, Artificial Sequence
  Description of Artificial Sequence Synthetic peptide
GSGEGRGSLLTCGDVEENPGP

SEQ ID NO: 132 - protein, 30 aa, Artificial Sequence
  Description of Artificial Sequence Synthetic polypeptide
MGGAAARLGAVILFVVIVGLHGVRGKYALA

SEQ ID NO: 133 - protein, 7 aa, Artificial Sequence
  Description of Artificial Sequence Synthetic peptide
DYDDDDK

SEQ ID NO: 134 - protein, 63 aa, Artificial Sequence
  Description of Artificial Sequence Synthetic polypeptide
GTLEIEAAFLERENTALETRVAELRQRVQRLRNRVSQYRTRYGPLGGGGSGGGGSGGGGSGNS

(19) United States
(12) Patent Application Publication
(10) Pub. No.: US 2024/0226207 A1
(43) Pub. Date: Jul. 11, 2024

(54) UNIVERSAL RETARGETING OF ONCOLYTIC HSV

(71) Applicant: Janssen Biotech, Inc., Horsham, PA (US)

(72) Inventors: David Baillat, Washington, DC (US); Matthew Mulvey, Gaithersburg, MD (US)

(21) Appl. No.: 18/558,859

(22) PCT Filed: May 4, 2022
(86) PCT No.: PCT/IB2022/054111

§ 371 Date: Nov. 3, 2023

Related U.S. Application Data
(60) Provisional application No. 63/184,283, filed on May 5, 2021.

(51) Int. Cl. A61K 35/763; A61K 47/68; A61P 35/00; C07K 16/14; C07K 16/18; C07K 16/28; C07K 16/30; C12N 15/86

(52) U.S. Cl. CPC A61K 35/763; A61K 47/6897; A61P 35/00; C07K 16/14; C07K 16/18; C07K 16/28; C07K 16/2803; C07K 16/2833; C07K 16/3069; C12N 15/86; C07K 2317/31; C07K 2317/569; C07K 2317/622; C12N 2710/16622; C12N 2710/16632; C12N 2710/16643; C12N 2710/16671

(57) ABSTRACT

Provided herein are bispecific adaptor proteins and their use for retargeting oncolytic HSV to target cells, such as tumor cells.

### CROSS REFERENCE TO RELATED APPLICATION

This application claims the benefit of U.S. Provisional Application 63/184,283, filed on May 5, 2021, which is incorporated by reference herein in its entirety.

### FIELD OF THE INVENTION

This disclosure relates to bispecific adaptor proteins and their use for retargeting oncolytic HSV to target cells, such as tumor cells.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Feb. 28, 2022, is named JBI6460WOPCT1_SeqListing.txt and is 192,512 bytes in size.

### BACKGROUND OF THE INVENTION

Oncolytic herpes simplex viruses (oHSV) are being extensively investigated for treatment of solid tumors. As a group, they pose many advantages over traditional cancer therapies (Markert J M et al., Genetically engineered HSV in the treatment of glioma: a review. Rev Med Virol. 2000 January-February; 10(1): 17-30; Russell S J et al., Oncolytic virotherapy. Nat Biotechnol. 2012 Jul. 10; 30(7):658-70; and Shen Y et al., Herpes simplex virus 1 (HSV-1) for cancer treatment. Cancer Gene Ther. 2006 November; 13(11):975-92). Specifically, oHSV usually embody a mutation that makes them susceptible to inhibition by some aspect of innate immunity. As a consequence they replicate in cancer cells in which one or more innate immune responses to infection are compromised but not in normal cells in which the innate immune responses are intact. oHSV are usually delivered directly into the tumor mass in which the virus can replicate. Because it is delivered to the target tissue rather than systemically, there are no side effect characteristics of anti-cancer drugs. Viruses characteristically induce adaptive immune responses that curtail their ability to be administered multiple times. oHSV has been administered to tumors multiple times without evidence of loss of potency or induction of adverse reaction such as inflammatory responses. HSV are large DNA viruses capable of incorporating into their genomes foreign DNA and to regulate the expression of these gene on administration to tumors. The foreign genes suitable for use with oHSV are those that help to induce an adaptive immune response to the tumor.

The defect in overcoming the cellular innate immune response determines the range of tumors in which the virus exhibits its oncolytic oHSV as an anti-cancer agent. The more extensive the deletions the more restrictive is the range of cancer cells in which the oHSV is effective depends on the function of the deleted viral gene. Most newer oHSV incorporate at least one cellular gene to bolster its anti-cancer activity (Cheema T A et al., Multifaceted oncolytic virus therapy for glioblastoma in an immunocompetent cancer stem cell model. Proc Natl Acad Sci USA. 2013 Jul. 16; 110(29): 12006-11; Goshima F et al., Oncolytic viral therapy with a combination of HF10, a herpes simplex virus type 1 variant and granulocyte-macrophage colony-stimulating factor for murine ovarian cancer. Int J Cancer. 2014 Jun. 15; 134(12):2865-77; Markert J M et al., Preclinical evaluation of a genetically engineered herpes simplex virus expressing interleukin-12. J Virol. 2012 May; 86(9):5304-13; and Walker J D et al., Oncolytic herpes simplex virus 1 encoding 15-prostaglandin dehydrogenase mitigates immune suppression and reduces ectopic primary and metastatic breast cancer in mice. J Virol. 2011 July; 85(14):7363-71).

It is convenient to consider separately the structure of the oHSV referred to as the backbone and the foreign genes appropriate for insertion into the backbone. As noted above the structure of the backbone determines the range of susceptible cancers. The foreign genes cause the host to see the cancer cells as legitimate targets of adaptive immune response.

The HSV genome consists of two covalently linked components, designated L and S. Each component consists of unique sequences (UL for the L component, US for the S component) flanked by inverted repeats. The inverted repeats of the L component are designated as ab and b′a′. The inverted repeats of the S component are designated as a′c′ and ca. Inverted repeats b′a′ and a′c′ constitute an internal inverted repeat region. The inverted repeats regions of both L and S components are known to contain two copies of five genes encoding proteins designated ICP0, ICP4, ICP34.5, ORF P and ORF O, respectively and large stretches of DNA that are transcribed but do not encode proteins.

Historically the viruses tested in cancer patients fall into 3 different designs. The first one was based on the evidence that deletion of the ICP34.5 gene significantly attenuated the virus (Andreansky S et al., Evaluation of genetically engineered herpes simplex viruses as oncolytic agents for human malignant brain tumors. Cancer Res. 1997 Apr. 15; 57(8):1502-9; Chou J et al., Association of a M(r) 90,000 phosphoprotein with protein kinase PKR in cells exhibiting enhanced phosphorylation of translation initiation factor eIF-2 alpha and premature shutoff of protein synthesis after infection with gamma 134.5-mutants of herpes simplex virus 1. Proc Natl Acad Sci USA. 1995 Nov. 7; 92(23): 10516-20; Chou J et al., Mapping of herpes simplex virus-1 neurovirulence to gamma 134.5, a gene nonessential for growth in culture. Science. 1990 Nov. 30; 250(4985): 1262-6; and Chou J et al., The gamma 1(34.5) gene of herpes simplex virus 1 precludes neuroblastoma cells from triggering total shutoff of protein synthesis characteristic of programed cell death in neuronal cells. Proc Natl Acad Sci USA. 1992 Apr. 15; 89(8):3266-70). To ensure its safety for treatment of malignant glioblastomas, G207, the first virus tested in patients was further attenuated by an additional mutation in the gene encoding the viral ribonucleotide reductase (Mineta T et al., Attenuated multi-mutated herpes simplex virus-1 for the treatment of malignant gliomas. Nat Med. 1995 September; 1(9):938-43). G207 carrying mutations in both the ICP34.5 and the ribonucleotide reductase genes was too attenuated and was shut off in cancer cells expressing a wild-type protein kinase R (Smith K D et al., Activated MEK suppresses activation of PKR and enables efficient replication and in vivo oncolysis by Deltagamma(1)34.5 mutants of herpes simplex virus 1. J Virol. 2006 February; 80(3):1110-20).

The second design was based on the demonstration that if a viral protein designated US11 is expressed early in infection it compensates in part for the absence of ICP34.5 and recoups ability to grow in cells expressing a wild-type protein kinase R (Mulvey et al., A herpesvirus ribosome-associated, RNA-binding protein confers a growth advantage upon mutants deficient in a GADD34-related function, J Virol. 1999 April; 73(4):3375-85). The design of the backbone of this virus follows that published by Cassady et al in that the US12 gene and the promoter of US11 are deleted (Cassady K A et al., The herpes simplex virus US11 protein effectively compensates for the gamma1(34.5) gene if present before activation of protein kinase R by precluding its phosphorylation and that of the alpha subunit of eukaryotic translation initiation factor 2. J Virol. 1998 November; 72(11):8620-6; Cassady K A et al., The second-site mutation in the herpes simplex virus recombinants lacking the gamma134.5 genes precludes shutoff of protein synthesis by blocking the phosphorylation of eIF-2alpha. J Virol. 1998 September; 72(9): 7005-11; and Mulvey M et al., A herpesvirus ribosome-associated, RNA-binding protein confers a growth advantage upon mutants deficient in a GADD34-related function. J Virol. 1999 April; 73(4):3375-85). As a consequence US11 is expressed as an immediate early gene rather than as a late gene. The FDA approved oHSV talimogene laherparepvec (previously known as OncoVexGM-CSF) utilizes this backbone design and further encodes the human GM-CSF gene under CMV promoter control (Liu et al., ICP34.5 deleted herpes simplex virus with enhanced oncolytic, immune stimulating, and anti-tumour properties, Gene Ther. 2003 February; 10(4):292-303).

The backbone of the third virus initially designated R7020 and later renamed NV1020 was the result of modifications of a spontaneous mutant that was initially tested as a live attenuated virus vaccine (Meignier B et al., In vivo behavior of genetically engineered herpes simplex viruses R7017 and R7020: construction and evaluation in rodents. J Infect Dis. 1988 September; 158(3):602-14 and Meignier B et al., Virulence of and establishment of latency by genetically engineered deletion mutants of herpes simplex virus 1. Virology. 1988 January; 162(1):251-4). This mutant lacked the internal inverted repeats (consisting of b′a′ and a′c′, encoding one copy of the genes ICP0, ICP4, ICP34.5, ORF P and ORF 0) and the genes encoding UL56 and UL24. In addition it contained bacterial sequences and since it was intended as a vaccine it also contained the genes encoding several HSV-2 glycoproteins. R7020 was extensively tested in patients with liver metastases from colon cancer. In addition it was tested in; head and neck epithelial squamous cell carcinoma and prostate adenocarcinoma xenografts in athymic nude mice and in bladder tumor models (Sze D Y et al., Response to intra-arterial oncolytic virotherapy with the herpes virus NV1020 evaluated by [18F]fluorodeoxyglucose positron emission tomography and computed tomography. Hum Gene Ther. 2012 January; 23(1):91-7; Cozzi P J et al., Intravesical oncolytic viral therapy using attenuated, replication-competent herpes simplex viruses G207 and Nv1020 is effective in the treatment of bladder cancer in an orthotopic syngeneic model. FASEB J. 2001 May; 15(7): 1306-8; Currier M A et al., Widespread intratumoral virus distribution with fractionated injection enables local control of large human rhabdomyosarcoma xenografts by oncolytic herpes simplex viruses. Cancer Gene Ther. 2005 April; 12(4):407-16; Fong Y et al., A herpes oncolytic virus can be delivered via the vasculature to produce biologic changes in human colorectal cancer. Mol Ther. 2009 February; 17(2):389-94; Geevarghese S K et al., Phase I/II study of oncolytic herpes simplex virus NV1020 in patients with extensively pretreated refractory colorectal cancer metastatic to the liver. Hum Gene Ther. 2010 September; 21(9): 1119-28; Kelly K et al., Attenuated multimutated herpes simplex virus-1 effectively treats prostate carcinomas with neural invasion while preserving nerve function. FASEB J. 2008 June; 22(6): 1839-48; Kemeny N et al., Phase I, open-label, dose-escalating study of a genetically engineered herpes simplex virus, NV1020, in subjects with metastatic colorectal carcinoma to the liver. Hum Gene Ther. 2006 December; 17(12): 1214-24; and Wong R J et al., Effective treatment of head and neck squamous cell carcinoma by an oncolytic herpes simplex virus. J Am Coll Surg. 2001 July; 193(1):12-21).

Entry of HSV into a target cell is a multistep process, requiring complex interactions and conformational changes of viral glycoproteins gD, gH/gL, gC and gB. These glycoproteins constitute the virus envelope which is the most external structure of the HSV particle and consists of a membrane. For cell entry, gC and gB mediate the first attachment of the HSV particle to cell surface heparan sulphate. Thereafter, a more specific interaction of the virus with the target cells occurs in that gD binds to at least two alternative cellular receptors, being nectin-1 (human: HveC) and HVEM (also known as HveA), causing conformational changes in gD that initiates a cascade of events leading to virion-cell membrane fusion. Thereby, the intermediate protein gH/gL (a heterodimer) is activated which triggers gB to catalyze membrane fusion.

In the current art, genetically engineered o-HSVs have been developed, which exhibit a highly specific tropism for the tumor cells, and are otherwise not attenuated. This approach has been defined as retargeting of HSV tropism to tumor-specific receptors.

The retargeting of HSV to cancer-specific receptors entails genetic modifications of gD, such that it harbors heterologous sequences which encode a specific ligand. Upon infection with the recombinant virus, progeny viruses are formed which carry in their envelope the chimeric gD-ligand glycoprotein, in place of wildtype gD. The ligand interacts with a molecule specifically expressed on the selected cell and enables entry of the recombinant o-HSV into the selected cell. Examples of ligands that have been successfully used for retargeting of HSV are IL13α, uPaR, a single chain antibody to HER2 and a single chain antibody to EGFR.

While retargeting entails that the recombinant virus is targeted to a selected cell, retargeting does not prevent that the recombinant virus is still capable of targeting its natural cellular receptors, resulting in infection and killing of a body's cells. In order to prevent binding of a herpesvirus to its natural receptors and killing of a body's normal cells, attempts have been made to reduce the binding to natural receptors. This is termed “detargeting”, which means that the recombinant herpesvirus has a reduced or no binding capability to a natural receptor of the unmodified herpesvirus, whereby the term “reduced” is used in comparison to the same herpesvirus with no such binding reducing modifications. This has the effect that normal cells are not infected or infected to a reduced extent and, thus, normal cells are not killed or less normal cells are killed. Such detargeted herpesvirus has reduced harmful activities by infecting less or not normal cells and increased beneficial activities by killing diseased cells.

While the art knows methods for retargeting of HSV to disease-specific receptors, these HSVs with the capability of being retargeted need to be propagated so that they can be produced in high amounts and are available as pharmaceuticals for treating diseases. In view of the fact that, for reasons of safety, the cells for propagation and production of the HSVs should not be diseased cells, so as to avoid the introduction of material such as DNA, RNA and/or protein of the diseased cells such as tumor cells in humans, the HSVs need to comprise additional modifications for enabling the HSVs of infecting “safe” cells which do not produce components which are harmful to humans for propagation and production of the HSVs.

The invention disclosed herein provides a system, wherein the recombinant HSVs can be propagated safely, de-targeted from normal cells, and retargeted to diseased (e.g. tumor) cells effectively.

### BRIEF SUMMARY OF THE INVENTION

Provided herein is a method of retargeting a recombinant herpes simplex virus (HSV) to a tumor cell expressing a TAA, the method comprising administering to a subject having the tumor cell, (a) the recombinant HSV, wherein the recombinant HSV comprises a nucleotide sequence encoding a heterologous ligand peptide and (b) an isolated bispecific adaptor protein, wherein the bispecific adaptor protein comprises a first binding domain with binding specificity to the heterologous ligand peptide expressed by the recombinant HSV and a second binding domain with binding specificity to the TAA expressed by the tumor cell, wherein, the first binding domain of the bispecific adaptor protein binds the heterologous ligand peptide expressed by the recombinant HSV and the second binding domain of the bispecific adaptor protein binds the TAA expressed by the tumor cell, thereby retargeting the recombinant HSV to the tumor cell.

In one embodiment of the method, the nucleotide sequence encoding the heterologous ligand peptide is inserted into the recombinant HSV by inserting into or replacing a portion of the nucleotide sequence encoding the wild type glycoprotein D (gD).

In a further embodiment of the method, the nucleotide sequence encoding the heterologous ligand peptide is inserted into the recombinant HSV replacing a nucleotide sequence encoding the amino acids 6-38 of wild type glycoprotein D (gD).

In a yet further embodiment of the method, the first binding domain of the bispecific adaptor protein comprises an antigen binding fragment with binding specificity to the heterologous peptide expressed by the recombinant HSV.

In a yet further embodiment of the method, the antigen binding fragment with binding specificity to the heterologous peptide is selected from the group consisting of single chain variable region (scFv), single chain antibody VHH, and polypeptide DARPin.

In a yet further embodiment of the method, the second binding domain of the bispecific adaptor protein comprises an antigen binding fragment with binding specificity to the TAA expressed by the tumor cell.

In a yet further embodiment, the antigen binding fragment with binding specificity to the TAA is selected from the group consisting of scFv, single chain antibody VHH, and polypeptide DARPin.

In a yet further embodiment of the method, the heterologous ligand peptide expressed by the recombinant HSV comprises GCN4 transcription factor or a fragment thereof.

In a yet further embodiment of the methods, the GCN4 transcription factor or fragment thereof comprises the amino acid sequence of SEQ ID NO: 4.

In a yet further embodiment of the method, the first binding domain of the bispecific adaptor protein comprises an antigen binding fragment with binding specificity to the GCN4 transcription factor or a fragment thereof.

In a yet further embodiment of the method, the antigen binding fragment with binding specificity to the GCN4 transcription factor or a fragment thereof is an anti-GCN4 scFv comprising a heavy chain variable region (VH) comprised of HCDR1 (SEQ ID NO: 16), HCDR2 (SEQ ID NO: 17), and HCDR3 (SEQ ID NO: 18) and/or a light chain variable region (VL) comprised of LCDR1 (SEQ ID NO: 19), LCDR2 (SEQ ID NO: 20), and LCDR3 (SEQ ID NO: 21).

In a yet further embodiment of the method, the antigen binding fragment with binding specificity to the GCN4 transcription factor or a fragment thereof is an anti-GCN4 scFv comprising a VH having a polypeptide sequence at least 95%, or at least 96%, or at least 97%, or at least 98%, or at least 99%, or 100% identical to SEQ ID NO: 22 and/or a VL having a polypeptide sequence at least 95%, or at least 96%, or at least 97%, or at least 98%, or at least 99%, or 100% identical to SEQ ID NO: 23.

In a yet further embodiment of the method, the heterologous ligand peptide expressed by the recombinant HSV comprises La protein or a fragment thereof.

In a yet further embodiment of the method, the La protein or fragment thereof comprises the amino acid sequence of SEQ ID NO: 12.

In a yet further embodiment of the method, the first binding domain of the bispecific adaptor protein comprises an antigen binding fragment with binding specificity to the La protein or fragment thereof.

In a yet further embodiment of the method, the antigen binding fragment with binding specificity to the La protein or fragment thereof is an anti-La scFv comprising a VH comprised of HCDR1 (SEQ ID NO: 26), HCDR2 (SEQ ID NO: 27), and HCDR3 (SEQ ID NO: 28) and/or a VL comprised of LCDR1 (SEQ ID NO: 29), LCDR2 (SEQ ID NO: 30), and LCDR3 (SEQ ID NO: 31).

In a yet further embodiment of the method, the antigen binding fragment with binding specificity to the La protein or fragment thereof is an anti-La scFv comprising a VH having a polypeptide sequence at least 95%, or at least 96%, or at least 97%, or at least 98%, or at least 99%, or 100% identical to SEQ ID NO: 32 and/or a VL having a polypeptide sequence at least 95%, or at least 96%, or at least 97%, or at least 98%, or at least 99%, or 100% identical to SEQ ID NO: 33.

In a yet further embodiment of the method, the heterologous ligand peptide expressed by the recombinant HSV comprises a first leucine-zipper moiety and the first binding domain of the bispecific adaptor protein comprises a second leucin-zipper moiety, wherein the first and second leucine-zipper moieties can form a leucine-zipper dimer.

In a yet further embodiment of the method, the first leucine-zipper moiety is synthetic leucine-zipper moiety RE (SEQ ID NO: 6) and the second leucine-zipper moiety is synthetic leucine-zipper moiety ER (SEQ ID NO: 10), or, the first leucine-zipper moiety is synthetic leucine-zipper moiety ER (SEQ ID NO: 10) and the second leucine-zipper moiety is synthetic leucine-zipper moiety RE (SEQ ID NO: 6).

In a yet further embodiment of method, the TAA expressed by the tumor cell is selected from the group consisting of PSMA, TMEFF2, ROR1, KLK2, and HLA-G.

In a yet further embodiment of the method, the TAA expressed by the tumor cell is PSMA, and wherein the second binding domain of the bispecific adaptor protein comprises an antigen binding fragment with binding specificity to PSMA.

In a yet further embodiment of the method, the antigen binding fragment with binding specificity to PSMA is an anti-PSMA VHH comprising HCDR1 (SEQ ID NO: 35), HCDR2 (SEQ ID NO: 36), and HCDR3 (SEQ ID NO: 37).

In a yet further embodiment of the method, the antigen binding fragment with binding specificity to PSMA is an anti-PSMA VHH comprising a polypeptide sequence at least 95%, or at least 96%, or at least 97%, or at least 98%, or at least 99%, or 100% identical to SEQ ID NO: 38.

In a yet further embodiment of the method, the antigen binding fragment with binding specificity to PSMA is an anti-PSMA VHH comprising HCDR1 (SEQ ID NO: 39), HCDR2 (SEQ ID NO: 40), and HCDR3 (SEQ ID NO: 41).

In a yet further embodiment of the method, the antigen binding fragment with binding specificity to PSMA is an anti-PSMA VHH comprising a polypeptide sequence at least 95%, or at least 96%, or at least 97%, or at least 98%, or at least 99%, or 100% identical to SEQ ID NO: 42.

In a yet further embodiment of the method, the antigen binding fragment with binding specificity to PSMA is an anti-PSMA scFv comprising a VH comprised of HCDR1 (SEQ ID NO: 43), HCDR2 (SEQ ID NO: 44), and HCDR3 (SEQ ID NO: 45) and/or a VL comprised of LCDR1 (SEQ ID NO: 46), LCDR2 (SEQ ID NO: 47), and LCDR3 (SEQ ID NO: 48).

In a yet further embodiment of the method, the antigen binding fragment with binding specificity to PSMA is an the anti-PSMA scFv comprising a VH having a polypeptide sequence at least 95%, or at least 96%, or at least 97%, or at least 98%, or at least 99%, or 100% identical to SEQ ID NO: 49 and/or a VL having a polypeptide sequence at least 95%, or at least 96%, or at least 97%, or at least 98%, or at least 99%, or 100% identical to SEQ ID NO: 50.

In a yet further embodiment of the method, the TAA expressed by the tumor cell is TMEFF2, and wherein the second binding domain of the bispecific adaptor protein comprises an antigen binding fragment with binding specificity to TMEFF2.

In a yet further embodiment of the method, the antigen binding fragment with binding specificity to TMEFF2 is an anti-TMEFF2 scFv comprising a VH comprised of HCDR1 (SEQ ID NO: 53), HCDR2 (SEQ ID NO: 54), and HCDR3 (SEQ ID NO: 55) and/or a VL comprised of LCDR1 (SEQ ID NO: 56), LCDR2 (SEQ ID NO: 57), and LCDR3 (SEQ ID NO: 58).

In a yet further embodiment of the method, the antigen binding fragment with binding specificity to TMEFF2 is an anti-TMEFF2 scFv comprising a VH having a polypeptide sequence at least 95%, or at least 96%, or at least 97%, or at least 98%, or at least 99%, or 100% identical to SEQ ID NO: 59 and/or a VL having a polypeptide sequence at least 95%, or at least 96%, or at least 97%, or at least 98%, or at least 99%, or 100% identical to SEQ ID NO: 60.

In a yet further embodiment of the method, the antigen binding fragment with binding specificity to TMEFF2 is an anti-TMEFF2 scFv comprising a VH comprised of HCDR1 (SEQ ID NO: 61), HCDR2 (SEQ ID NO: 62), and HCDR3 (SEQ ID NO: 63) and/or a VL comprised of LCDR1 (SEQ ID NO: 64), LCDR2 (SEQ ID NO: 65), and LCDR3 (SEQ ID NO: 66).

In a yet further embodiment of the method, the antigen binding fragment with binding specificity to TMEFF2 is an anti-TMEFF2 scFv comprising a VH having a polypeptide sequence at least 95%, or at least 96%, or at least 97%, or at least 98%, or at least 99%, or 100% identical to SEQ ID NO: 67 and/or a VL having a polypeptide sequence at least 95%, or at least 96%, or at least 97%, or at least 98%, or at least 99%, or 100% identical to SEQ ID NO: 68.

In a yet further embodiment of the method, the TAA expressed by the tumor cell is KLK2, and wherein the second binding domain of the bispecific adaptor protein comprises an antigen binding fragment with binding specificity to KLK2.

In a yet further embodiment of the method, the antigen binding fragment with binding specificity to KLK2 is an anti-KLK2 scFv comprising a VH comprised of HCDR1 (SEQ ID NO: 72), HCDR2 (SEQ ID NO: 73), and HCDR3 (SEQ ID NO: 74) and/or a VL comprised of LCDR1 (SEQ ID NO: 75), LCDR2 (SEQ ID NO: 76), and LCDR3 (SEQ ID NO: 77).

In a yet further embodiment of the method, the antigen binding fragment with binding specificity to KLK2 is an anti-KLK2 scFv comprising a VH having a polypeptide sequence at least 95%, or at least 96%, or at least 97%, or at least 98%, or at least 99%, or 100% identical to SEQ ID NO: 78 and/or a VL having a polypeptide sequence at least 95%, or at least 96%, or at least 97%, or at least 98%, or at least 99%, or 100% identical to SEQ ID NO: 79.

In a yet further embodiment of the method, the antigen binding fragment with binding specificity to KLK2 is an anti-KLK2 scFv comprising a VH having a polypeptide sequence at least 95%, or at least 96%, or at least 97%, or at least 98%, or at least 99%, or 100% identical to SEQ ID NO: 80 and/or a VL having a polypeptide sequence at least 95%, or at least 96%, or at least 97%, or at least 98%, or at least 99%, or 100% identical to SEQ ID NO: 81.

In a yet further embodiment of the method, the TAA expressed by the tumor cell is HLA-G, and wherein the second binding domain of the bispecific adaptor protein comprises an antigen binding fragment with binding specificity to HLA-G.

In a yet further embodiment of the method, the TAA expressed by the tumor cell is ROR1, and wherein the second binding domain of the bispecific adaptor protein comprises an antigen binding fragment with binding specificity to ROR1.

In a yet further embodiment of the method, the antigen binding fragment with binding specificity to ROR1 is a polypeptide DARPin having a polypeptide sequence at least 95%, or at least 96%, or at least 97%, or at least 98%, or at least 99%, or 100% identical to SEQ ID NO: 86.

Further provided herein is a method of treating a cancer in a subject, wherein a TAA is expressed by the cancer cell, the method comprising administering to the subject, (a) a recombinant HSV, wherein the recombinant HSV comprises a nucleotide sequence encoding a heterologous ligand peptide and (b) an isolated bispecific adaptor protein, wherein the bispecific adaptor protein comprises a first binding domain with binding specificity to the heterologous ligand peptide expressed by the recombinant HSV and a second binding domain with binding specificity to the TAA expressed by the cancer cell, wherein, the first binding domain of the bispecific adaptor protein binds the heterologous ligand peptide expressed by the recombinant HSV and the second binding domain of the specific adaptor protein binds the TAA expressed by the cancer cell, thereby causing oncolysis of the cancer cell.

In one embodiment of the method of treating, the nucleotide sequence encoding the heterologous ligand peptide is inserted into the recombinant HSV by inserting into or replacing a portion of the nucleotide sequence encoding the wild type glycoprotein D (gD).

In a further embodiment of the method of treating, the nucleotide sequence encoding the heterologous ligand peptide is inserted into the recombinant HSV replacing a nucleotide sequence encoding the amino acids 6-38 of wild type gD.

Yet further provided herein is a bispecific adaptor protein for retargeting a recombinant HSV to a tumor cell, wherein the bispecific adaptor protein comprises a first binding domain with binding specificity to a heterologous ligand peptide expressed by the recombinant HSV and a second binding domain with binding specificity to a TAA expressed by the tumor cell.

In one embodiment of the bispecific adaptor protein, each of the first and second binding domains of the bispecific adaptor protein comprises an antigen binding fragment.

In a further embodiment of the bispecific adaptor protein, the antigen binding fragment is selected from the group consisting of scFv, single chain antibody VHH, and polypeptide DARPin.

In a yet further embodiment of the bispecific adaptor protein, the first binding domain of the bispecific adaptor protein comprises an antigen binding fragment with binding specificity to GCN4 transcription factor or a fragment thereof.

In a yet further embodiment of the bispecific adaptor protein, the antigen binding fragment with binding specificity to GCN4 transcription factor or a fragment thereof is an anti-GCN4 scFv comprising a VH comprised of HCDR1 (SEQ ID NO: 16), HCDR2 (SEQ ID NO: 17), and HCDR3 (SEQ ID NO: 18) and/or a VL comprised of LCDR1 (SEQ ID NO: 19), LCDR2 (SEQ ID NO: 20), and LCDR3 (SEQ ID NO: 21).

In a yet further embodiment of the bispecific adaptor protein, the antigen binding fragment with binding specificity to GCN4 transcription factor or a fragment thereof is an anti-GCN4 scFv comprising a VH having a polypeptide sequence at least 95%, or at least 96%, or at least 97%, or at least 98%, or at least 99%, or 100% identical to SEQ ID NO: 22 and/or a VL having a polypeptide sequence at least 95%, or at least 96%, or at least 97%, or at least 98%, or at least 99%, or 100% identical to SEQ ID NO: 23.

In a yet further embodiment of the bispecific adaptor protein, the first binding domain of the bispecific adaptor protein comprises an antigen binding fragment with binding specificity to La protein or fragment thereof.

In a yet further embodiment of the bispecific adaptor protein, the antigen binding fragment with binding specificity to La protein or fragment thereof is an anti-La scFv comprising a VH comprised of HCDR1 (SEQ ID NO: 26), HCDR2 (SEQ ID NO: 27), and HCDR3 (SEQ ID NO: 28) and/or a VL comprised of LCDR1 (SEQ ID NO: 29), LCDR2 (SEQ ID NO: 30), and LCDR3 (SEQ ID NO: 31).

In a yet further embodiment of the bispecific adaptor protein, the antigen binding fragment with binding specificity to La protein or fragment thereof is an anti-La scFv comprising a VH having a polypeptide sequence at least 95%, or at least 96%, or at least 97%, or at least 98%, or at least 99%, or 100% identical to SEQ ID NO: 32 and/or a VL having a polypeptide sequence at least 95%, or at least 96%, or at least 97%, or at least 98%, or at least 99%, or 100% identical to SEQ ID NO: 33.

In a yet further embodiment of the bispecific adaptor protein, the first binding domain of the bispecific adaptor protein comprises a leucine-zipper moiety.

In a yet further embodiment of the bispecific adaptor protein, the leucine-zipper moiety is synthetic leucine-zipper moiety RE (SEQ ID NO: 6) or synthetic leucine-zipper moiety ER (SEQ ID NO: 10).

In a yet further embodiment of the bispecific adaptor protein, the TAA expressed by the tumor cell is PSMA, and wherein the second binding domain of the bispecific adaptor protein comprises an antigen binding fragment with binding specificity to PSMA.

In a yet further embodiment of the bispecific adaptor protein, the antigen binding fragment with binding specificity to PSMA is anti-PSMA VHH comprising HCDR1 (SEQ ID NO: 35), HCDR2 (SEQ ID NO: 36), and HCDR3 (SEQ ID NO: 37).

In a yet further embodiment of the bispecific adaptor protein, the antigen binding fragment with binding specificity to PSMA is an anti-PSMA VHH comprising a polypeptide sequence at least 95%, or at least 96%, or at least 97%, or at least 98%, or at least 99%, or 100% identical to SEQ ID NO: 38.

In a yet further embodiment of the bispecific adaptor protein, the antigen binding fragment with binding specificity to PSMA is an anti-PSMA VHH comprising HCDR1 (SEQ ID NO: 39), HCDR2 (SEQ ID NO: 40), and HCDR3 (SEQ ID NO: 41).

In a yet further embodiment of the bispecific adaptor protein, the antigen binding fragment with binding specificity to PSMA is an anti-PSMA VHH comprising a polypeptide sequence at least 95%, or at least 96%, or at least 97%, or at least 98%, or at least 99%, or 100% identical to SEQ ID NO: 42.

In a yet further embodiment of the bispecific adaptor protein, the antigen binding fragment with binding specificity to PSMA is an anti-PSMA scFv comprising a VH comprised of HCDR1 (SEQ ID NO: 43), HCDR2 (SEQ ID NO: 44), and HCDR3 (SEQ ID NO: 45) and/or a VL comprised of LCDR1 (SEQ ID NO: 46), LCDR2 (SEQ ID NO: 47), and LCDR3 (SEQ ID NO: 48).

In a yet further embodiment of the bispecific adaptor protein, the antigen binding fragment with binding specificity to PSMA is an anti-PSMA scFv comprising a VH having a polypeptide sequence at least 95%, or at least 96%, or at least 97%, or at least 98%, or at least 99%, or 100% identical to SEQ ID NO: 49 and/or a VL having a polypeptide sequence at least 95%, or at least 96%, or at least 97%, or at least 98%, or at least 99%, or 100% identical to SEQ ID NO: 50.

In a yet further embodiment of the bispecific adaptor protein, the TAA expressed by the tumor cell is TMEFF2, and wherein the second binding domain of the bispecific adaptor protein comprises an antigen binding fragment with binding specificity to TMEFF2.

In a yet further embodiment of the bispecific adaptor protein, the antigen binding fragment with binding specificity to TMEFF2 is an anti-TMEFF2 scFv comprising a VH comprised of HCDR1 (SEQ ID NO: 53), HCDR2 (SEQ ID NO: 54), and HCDR3 (SEQ ID NO: 55) and/or a VL comprised of LCDR1 (SEQ ID NO: 56), LCDR2 (SEQ ID NO: 57), and LCDR3 (SEQ ID NO: 58).

In a yet further embodiment of the bispecific adaptor protein, the antigen binding fragment with binding specificity to TMEFF2 is an anti-TMEFF2 scFv comprising a VH having a polypeptide sequence at least 95%, or at least 96%, or at least 97%, or at least 98%, or at least 99%, or 100% identical to SEQ ID NO: 59 and/or a VL having a polypeptide sequence at least 95%, or at least 96%, or at least 97%, or at least 98%, or at least 99%, or 100% identical to SEQ ID NO: 60.

In a yet further embodiment of the bispecific adaptor protein, the antigen binding fragment with binding specificity to TMEFF2 is an anti-TMEFF2 scFv comprising a VH comprised of HCDR1 (SEQ ID NO: 61), HCDR2 (SEQ ID NO: 62), and HCDR3 (SEQ ID NO: 63) and/or a VL comprised of LCDR1 (SEQ ID NO: 64), LCDR2 (SEQ ID NO: 65), and LCDR3 (SEQ ID NO: 66).

In a yet further embodiment of the bispecific adaptor protein, the antigen binding fragment with binding specificity to TMEFF2 is an anti-TMEFF2 scFv comprising a VH having a polypeptide sequence at least 95%, or at least 96%, or at least 97%, or at least 98%, or at least 99%, or 100% identical to SEQ ID NO: 67 and/or a VL having a polypeptide sequence at least 95%, or at least 96%, or at least 97%, or at least 98%, or at least 99%, or 100% identical to SEQ ID NO: 68.

In a yet further embodiment of the bispecific adaptor protein, wherein the TAA expressed by the tumor cell is KLK2, and wherein the second binding domain of the bispecific adaptor protein comprises an antigen binding fragment with binding specificity to KLK2.

In a yet further embodiment of the bispecific adaptor protein, the antigen binding fragment with binding specificity to KLK2 is an anti-KLK2 scFv comprising a VH comprised of HCDR1 (SEQ ID NO: 72), HCDR2 (SEQ ID NO: 73), and HCDR3 (SEQ ID NO: 74) and/or a VL comprised of LCDR1 (SEQ ID NO: 75), LCDR2 (SEQ ID NO: 76), and LCDR3 (SEQ ID NO: 77).

In a yet further embodiment of the bispecific adaptor protein, the antigen binding fragment with binding specificity to KLK2 is an anti-KLK2 scFv comprising a VH having a polypeptide sequence at least 95%, or at least 96%, or at least 97%, or at least 98%, or at least 99%, or 100% identical to SEQ ID NO: 78 and/or a VL having a polypeptide sequence at least 95%, or at least 96%, or at least 97%, or at least 98%, or at least 99%, or 100% identical to SEQ ID NO: 79.

In a yet further embodiment of the bispecific adaptor protein, the antigen binding fragment with binding specificity to KLK2 is an anti-KLK2 scFv comprising a VH having a polypeptide sequence at least 95%, or at least 96%, or at least 97%, or at least 98%, or at least 99%, or 100% identical to SEQ ID NO: 80 and/or a VL having a polypeptide sequence at least 95%, or at least 96%, or at least 97%, or at least 98%, or at least 99%, or 100% identical to SEQ ID NO: 81.

In a yet further embodiment of the bispecific adaptor protein, the TAA expressed by the tumor cell is HLA-G, and wherein the second binding domain of the bispecific adaptor protein comprises an antigen binding fragment with binding specificity to HLA-G.

In a yet further embodiment of the bispecific adaptor protein, the TAA expressed by the tumor cell is ROR1, and wherein the second binding domain of the bispecific adaptor protein comprises an antigen binding fragment with binding specificity to ROR1.

In a yet further embodiment of the bispecific adaptor protein, the antigen binding fragment with binding specificity to ROR1 is a polypeptide DARPin having a polypeptide sequence at least 95%, or at least 96%, or at least 97%, or at least 98%, or at least 99%, or 100% identical to SEQ ID NO: 86.

Yet further provided herein is an isolated nucleic acid comprising a polynucleotide sequence encoding the isolated bispecific adaptor protein as described above.

Yet further provided herein is an isolated vector comprising the isolated nucleic acid sequence as described above.

Yet further provided herein is a recombinant host cell comprising the isolated vector as described above.

Yet further provided herein is a kit comprising a recombinant HSV as described above and instructions for use of the recombinant HSV.

Yet further provided herein is a kit comprising an isolated bispecific adaptor protein as described above and instructions for use of the bispecific adaptor protein.

Yet further provided herein is a kit comprising a recombinant HSV as described above, an isolated adaptor protein as described above, and instructions for use.

Yet further provided herein is a recombinant HSV comprising a nucleotide sequence encoding a heterologous ligand peptide, wherein the heterologous ligand peptide comprises La protein or a fragment thereof, and wherein the nucleotide sequence encoding the heterologous ligand peptide is inserted into the recombinant HSV by inserting into or replacing a portion of replacing the wild type gD. In one embodiment, the nucleotide sequence encoding the heterologous ligand peptide is inserted into the recombinant HSV replacing a nucleotide sequence encoding the amino acid 6-38 of wild type gD. In a further embodiment, the La protein or fragment thereof comprises the amino acid sequence of SEQ ID NO: 12.

Yet further provided herein is a recombinant HSV comprising a nucleotide sequence encoding a heterologous ligand peptide, wherein the heterologous ligand peptide comprises a leucine-zipper moiety, and wherein the nucleotide sequence encoding the heterologous ligand peptide is inserted into the recombinant HSV by inserting into or replacing a portion of replacing the wild type gD. In one embodiment, the nucleotide sequence encoding the heterologous ligand peptide is inserted into the recombinant HSV replacing a nucleotide sequence encoding the amino acid 6-38 of wild type gD. In a further embodiment, the leucine-zipper moiety is synthetic leucine-zipper moiety RE (SEQ ID NO: 6) or synthetic leucine-zipper moiety ER (SEQ ID NO: 10).

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows that due to its bispecificity, the bispecific adaptor protein disclosed herein retargets the recombinant HSV to tumor cells.

FIG. 2 shows various embodiments of the bispecific adaptor proteins disclosed herein. FIG. 2 discloses“(GGGGS)₄” as SEQ ID NO: 15 and “GGGGS” as SEQ ID NO: 124.

FIG. 3 shows the genome structure of a GCN4-retargeted recombinant HSV. FIG. 3 discloses SEQ ID NO: 5.

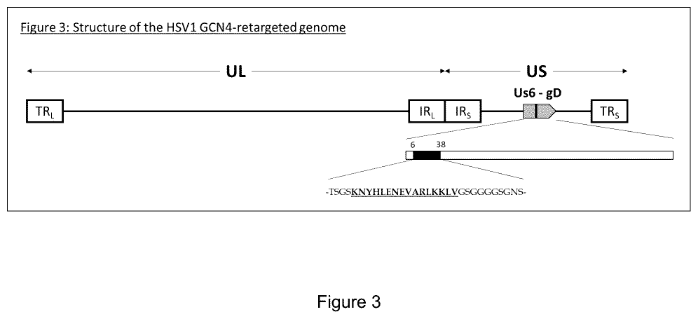

FIG. 4 shows a RE/ER-retargeted recombinant HSV and it is being retargeted to tumor cells by a bispecific adaptor protein.

FIG. 5 shows the structure of RR12EE345L-(G₄S)₃-d6-38gD (ER/RE retargeted gD) and EE12RR345L-(G₄S)₃-hNectin1 (ER/RE-Nectin1) used for HSV1 retargeting using the ER/RE leucine zipper pair. ER/RE retargeted gD was obtained by replacing AA6-38 of gD by the RR12EE345L leucine zipper and a (G₄S)₃ linker (SEQ ID NO: 126). ER/RE-Nectin1 was obtained by replacing the first Ig-like domain of hNectin1 (AA31-145 of UniProtKB—Q15223 (NECT1_HUMAN)) by EE12RR345L leucine zipper and a (G₄S)₃ linker (SEQ ID NO: 126). FIG. 5 discloses SEQ ID NOs. 8 and 134, respectively, in order of appearance.

FIG. 6 shows the infection of Vero-H6-nectin1 and B16-F10-H6-nectin1 cells by GCN4-retargeted virus (MOI=1). Parental Vero and B16-F10 cells were used as a negative control for retargeting. An oHSV1 expressing GFP was used as a positive control for infection on Vero cells (left panel).

FIG. 7A shows the expression of the PSMA-H6 bispecific adaptor proteins in supernatants of transiently transfected HEK293T, 48 hr post transfection. The bispecific adaptor proteins were detected with an anti-myc tag antibody. The supernatant of un-transfected HEK293T cells was used as a negative control (mock).

FIG. 7B shows the expression of PSMA at the surface of the HEK293T-PSMA stable cell line analyzed by FACS. Parental HEK293T cell was used as a negative control.

FIG. 7C shows the infection of HEK293T-PSMA and LNCaP cells (PSMA+) by GCN4-retargeted virus (MOI=0.1) in the presence of PSMA-H6 bispecific adaptor proteins. Parental HEK293T and DU145 cells (PSMA-) were used as a negative control for retargeting. An oHSV1 expressing GFP was used as a positive control for infection (bottom panel).

FIG. 8A shows the expression of the TMEFF2-H6 bispecific adaptor proteins in supernatants of transiently transfected HEK293T 48 hr post transfection. The bispecific adaptor proteins were detected with an anti-myc tag antibody. The supernatant of un-transfected HEK293T cells was used as a control (mock).

FIG. 8B shows the expression of TMEFF2 at the surface of the Vero-TMEFF2 stable cell line (before and after cell sorting for TMEFF2 expression) analyzed by FACS. Parental Vero cells were used as a negative control.

FIG. 8C shows the infection of Vero-TMEFF2 and 22Rv1 cells (TMEFF2+) by GCN4-retargeted virus (MOI=0.1) in the presence of TMEFF2-H6 bispecific adaptor proteins. Parental Vero were used as negative control for retargeting. An oHSV1 expressing GFP was used as a positive control for infection. 22Rv1 cells were shown at 24 Hr and 72 hr infection to confirm the growth of the retargeted virus in presence of the bispecific adaptor proteins.

FIG. 9A shows the expression of the KLK2-H6 bispecific adaptor proteins in supernatants of transiently transfected HEK293T 48 hr post transfection. The bispecific adaptor proteins were detected with an anti-myc tag antibody. The supernatant of un-transfected HEK293T cells was used as a negative control (mock).

FIG. 9B shows the expression of KLK2 at the surface of the Vero-KLK2-nectin1 stable cell line (before and after cell sorting for FLAG-tag expression) analyzed by FACS. Parental Vero cells were used as a control.

FIG. 9C shows the infection of Vero-KLK2-nectin1 cells by GCN4-retargeted virus (MOI=0.1) in the presence of KLK2-H6 bispecific adaptor proteins. Parental Vero are used as a negative control for retargeting. An oHSV1 expressing GFP was used as a positive control for infection (bottom panel).

FIG. 10A shows the expression of the How-H6 bispecific adaptor protein in supernatant of transiently transfected HEK293T 48 hr post transfection. The bispecific adaptor protein was detected with an anti-myc tag antibody. The supernatant of un-transfected HEK293T cells was used as a negative control (mock).

FIG. 10B shows the expression of ROR1 at the surface of HEK293T cells analyzed by FACS (Solid: isotype, light grey: anti-ROR1).

FIG. 10C shows the infection of HEK293T cells by GCN4-retargeted virus (MOI=0.1) in the presence of the How-H6 bispecific adaptor protein. Parental 293T cells were used as a negative control for retargeting. An oHSV1 expressing GFP was used as a positive control for infection (top panel).

FIG. 11A shows the retargeting of RR12EE345L-(G₄S)₃-d6-38gD to EE12RR345L-(G₄S)₃-Nectin1 measured by in vitro fusion assay using a dual split reporter protein system (Kondo et al. JBC 2010, Ishikawa et al. Protein Eng Des Sel 2012) where the luciferase reporter activity is a measure of cell-cell fusion. The effector cells (HEK293T) were transfected with plasmids expressing HSV1 gH, gL, gB and RR12EE345L-(G₄S)₃-d6-38gD and cDSP while the target cells (HEK293T) were transfected with EE12RR345L-(G₄S)₃-Nectin1 and nDSP (Lane 2). The negative control (Lane 1) is identical to Lane 2 except that the plasmid expressing EE12RR345L-(G₄S)₃-Nectin1 was omitted.

FIG. 11B shows the retargeting of RR12EE345L-(G₄S)₃-d6-38gD to PSMA using B588LH-EE12RR345L bispecific adaptor measured by in vitro fusion assay using a dual split reporter protein system where the luciferase reporter activity is a measure of cell-cell fusion. The effector cells (HEK293T) were transfected with plasmids expressing HSV1 gH, gL, gB and RR12EE345L-(G₄S)₃-d6-38gD and cDSP. The target cells (HEK293T) were transfected with plasmid expressing PSMA, B588LH-EE12RR345L and nDSP (lane 5). The positive control (lane 3) used HEK293T transfected with plasmids expressing HSV1 gH, gL, gB and B588LH-d6-38gD and cDSP as effector cells and HEK293T cells transfected with plasmids expressing PSMA and nDSP as target cells. The negative control (lane 4) is identical to lane 5 except that the plasmid expressing the bispecific adaptor B588LH-EE12RR345L was omitted.

FIG. 11C shows the retargeting of RR12EE345L-(G₄S)₃-d6-38gD to KLK2 using KL2B359LH-EE12RR345L bispecific adaptor measured by in vitro fusion assay using a dual split reporter protein system where the luciferase reporter activity is a measure of cell-cell fusion. The effector cells (HEK293T) were transfected with plasmids expressing HSV1 gH, gL, gB and RR12EE345L-(G₄S)₃-d6-38gD and cDSP. The target cells (HEK293T) were transfected with plasmid expressing KLK2-Nectin1, KL2B359LH-EE12RR345L and nDSP (lane 8). The positive control (lane 6) used HEK293T cells transfected with plasmids expressing HSV1 gH, gL, gB and KL2B359LH-d6-38gD and cDSP as effector cells and HEK293T cells were transfected with plasmids expressing KLK2-Nectin1 and nDSP as target cells. The negative control (lane 7) is identical to lane 8 except that the plasmid expressing the bispecific adaptor KL2B359LH-EE12RR345L was omitted.

FIG. 11D shows the retargeting of RR12EE345L-(G₄S)₃-d6-38gD to TMEFF2 using TMEF9LH-EE12RR345L bispecific adaptor measured by in vitro fusion assay using a dual split reporter protein system where the luciferase reporter activity is a measure of cell-cell fusion. The effector cells (HEK293T) were transfected with plasmids expressing HSV1 gH, gL, gB and RR12EE345L-(G₄S)₃-d6-38gD and cDSP. The target cells (HEK293T) were transfected with plasmid expressing TMEFF2, TMEF9LH-EE12RR345L and nDSP (lane 11). The positive control (lane 9) used HEK293T cells transfected with plasmids expressing HSV1 gH, gL, gB and TMEF9LH-d6-38gD and cDSP as effector cells and HEK293T cells were transfected with plasmids expressing TMEFF2 and nDSP as target cells. The negative control (lane10) is identical to lane 11 except that the plasmid expressing the bispecific adaptor TMEF9LH-EE12RR345L was omitted.

FIG. 12A shows the retargeting of La-d6-38gD to 5B9HL-Nectin1 measured by in vitro fusion assay using a dual split reporter protein system (Kondo et al. JBC 2010, Ishikawa et al. Protein Eng Des Sel 2012) where the luciferase reporter activity is a measure of cell-cell fusion. The effector cells (HEK293T) were transfected with plasmids expressing HSV1 gH, gL, gB and La-d6-38gD and cDSP while the target cells (HEK293T) were transfected with 5B9HL-Nectin1 and nDSP (Lane 2). The negative control (Lane 1) is identical to Lane 2 except that the plasmid expressing 5B9HL-Nectin1 was omitted.

FIG. 12B shows the retargeting of La-d6-38gD to PSMA using B588LH-5B9HL bispecific adaptor measured by in vitro fusion assay using a dual split reporter protein system where the luciferase reporter activity is a measure of cell-cell fusion. The effector cells (HEK293T) were transfected with plasmids expressing HSV1 gH, gL, gB and La-d6-38gD and cDSP. The target cells (HEK293T) were transfected with plasmid expressing PSMA, B588LH-5B9HL and nDSP (lane 5). The positive control (lane 3) used HEK293T transfected with plasmids expressing HSV1 gH, gL, gB and B588LH-d6-38gD and cDSP as effector cells and HEK293T cells transfected with plasmids expressing PSMA and nDSP as target cells. The negative control (lane 4) is identical to lane 5 except that the plasmid expressing the bispecific adaptor B588LH-5B9HL was omitted.

FIG. 12C shows the retargeting of La-d6-38gD to KLK2 using KL2B359LH-5B9HL bispecific adaptor measured by in vitro fusion assay using a dual split reporter protein system where the luciferase reporter activity is a measure of cell-cell fusion. The effector cells (HEK293T) were transfected with plasmids expressing HSV1 gH, gL, gB and La-d6-38gD and cDSP. The target cells (HEK293T) were transfected with plasmid expressing KLK2-Nectin1, KL2B359LH-5B9HL and nDSP (lane 8). The positive control (lane 6) used HEK293T cells transfected with plasmids expressing HSV1 gH, gL, gB and KL2B359LH-d6-38gD and cDSP as effector cells and HEK293T cells were transfected with plasmids expressing KLK2-Nectin1 and nDSP as target cells. The negative control (lane 7) is identical to lane 8 except that the plasmid expressing the bispecific adaptor KL2B359LH-5B9HL was omitted.

FIG. 12D shows the retargeting of La-d6-38gD to TMEFF2 using TMEF9LH-5B9HL bispecific adaptor measured by in vitro fusion assay using a dual split reporter protein system where the luciferase reporter activity is a measure of cell-cell fusion. The effector cells (HEK293T) were transfected with plasmids expressing HSV1 gH, gL, gB and La-d6-38gD and cDSP. The target cells (HEK293T) were transfected with plasmid expressing TMEFF2, TMEF9LH-5B9HL and nDSP (lane 11). The positive control (lane 9) used HEK293T cells transfected with plasmids expressing HSV1 gH, gL, gB and TMEF9LH-d6-38gD and cDSP as effector cells and HEK293T cells were transfected with plasmids expressing TMEFF2 and nDSP as target cells. The negative control (lane10) is identical to lane 11 except that the plasmid expressing the bispecific adaptor TMEF9LH-5B9HL was omitted.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., chimeric antigen receptors (CARs) and the isolated polynucleotides that encode them; isolated monoclonal or bispecific antibodies and antigen-binding fragments thereof and the nucleic acids that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds. and Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

The term “vector” means a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The vector polynucleotides may be DNA or RNA molecules or a hybrid of these. Exemplary vectors include, without limitation, plasmids, cosmids, phage vectors, and viral vectors. The term “expression vector” means a vector that can be utilized in a biological system or in a reconstituted biological system to direct the translation of a polypeptide encoded by a polynucleotide sequence present in the expression vector.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications.

“Heterologous,” as used herein, means a nucleotide or polypeptide sequence that is not found in the native nucleic acid or protein of a given organism, respectively. For example, in the context of a recombinant HSV of the present disclosure, a nucleic acid comprising a nucleotide sequence encoding a “heterologous” GCN4 transcription factor or fragment thereof is a nucleic acid that is not found naturally in HSV, i.e., the encoded GCN4 transcription factor or fragment thereof is not encoded by naturally-occurring HSV.

“Antigen binding fragment” or “antigen binding domain” refers to a portion of the protein that binds an antigen, e.g., an antibody or an epitope binding peptide. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins. Exemplary antigen binding fragments also include genetically engineered antibody mimetic proteins, such as DARPin.

### Recombinant (Retargeted) Herpes Simplex Virus (HSV)

Herpes simplex virus (HSV) is one of the many human and animal viruses that have been modified or adapted for oncolytic purpose. Several intrinsic properties of HSV make it an attractive candidate as an oncolytic agent. First, lytic infection by HSV usually kills target cells much more rapidly than infection by other DNA viruses. Rapid replication and spreading among target cells are vital properties allowing a virus to execute its full oncolytic potential in vivo, as the body's immune mechanism may be more likely to restrict the spread of slower growing viruses. Second, HSV has a wide tropism and oncolytic viruses derived from it can be applied therapeutically to many different types of tumors. In principle, this property should protect against the rapid development of resistance to virotherapy using HSV in contrast to other oncolytic viruses such as those derived from adenoviruses. Finally, effective anti-HSV medications such as acyclovir and famciclovir are readily available as safety measures in the event of undesired infection or toxicity from the virus.

The terms “herpes simplex virus (HSV)” and “oncolytic herpes simplex virus (oHSV)” are used interchangeably herein. The HSV used herein can selectively replicate within tumor cells, resulting in their destruction and in the production of progeny virions that can spread to adjacent tumor cells. Both serotypes of HSV, HSV-1 and HSV-2 can be used herein. In one embodiment, the HSV used herein is HSV-1. In a further embodiment, the HSV used herein may be selected from oncolytic HSVs including, without limitation, HSV1716 (aka Seprehvir), G207, G47Delta, Talimogene laherparepvec (aka OncoVexGM-CSF), NV1020, NV1023, NV1034, NV1042, rQNestin34.5, RP1, RP2, RP3, ONCR-148, ONCR-177, ONCR-152, ONCR-153, VG161, and other known HSVs, including those disclosed and taught in WO/2013/036795 (BeneVir Pharm, Inc.).

Glycoprotein D (gD) is a 55 kDa virion envelope glycoprotein which is essential for HSV entry into host cells and plays an essential role in herpesvirus infectivity. Upon entry of HSV into a cell, the interaction of gD with the heterodimer gH/gL is the critical event in an activation cascade involving the four glycoproteins gD, gH, gL, and gB, which are involved in HSV entry into a cell. The activation cascade starts with the binding of gD to one of its receptors, nectin-1, HVEM, and modified heparan sulfates, which is transmitted to gH/gL, and finally to gB. gB carries out the fusion of the HSV with the target cell membrane. The heterodimer gH/gL interacts with the profusion domain of gD which profusion domain is dislodged upon interaction of gD with one of its receptors during cell entry. gD comprises some specific regions which are responsible for HSV to be targeted to its natural receptors, such as nectin-1 and HVEM.

Disclosed herein is a recombinant HSV, in which a nucleotide sequence encoding all or part of the HVEM binding site and all or part of the nectin-1 binding site is deleted.

In one embodiment, the recombinant HSV has the nucleotide sequence encoding all or part of the HVEM binding site and all or part of the nectin-1 binding site deleted and replaced by a heterologous nucleotide sequence encoding a ligand peptide.

The full sequence of gD with signal peptide (underlined) is as follows:

(SEQ ID NO: 1)
MGGTAARLGAVILFVVIVGLHGVRGKYALADASLKMADPNRFRGKDLPV
LDQLTDPPGVRRVYHIQAGLPDPFQPPSLPITVYYAVLERACRSVLLNA
PSEAPQIVRGASEDVRKQPYNLTIAWFRMGGNCAIPITVMEYTECSYNK
SLGACPIRTQPRWNYYDSFSAVSEDNLGFLMHAPAFETAGTYLRLVKIN
DWTEITQFILEHRAKGSCKYALPLRIPPSACLSPQAYQQGVTVDSIGML
PRFIPENQRTVAVYSLKIAGWHGPKAPYTSTLLPPELSETPNATQPELA
PEDPEDSALLEDPVGTVAPQIPPNWHIPSIQDAATPYHPPATPNNMGLI
AGAVGGSLLAALVICGIVYWMHRRTRKAPKRIRLPHIREDDQPSSHQPL
FY

The mature protein of gD is as follows:

(SEQ ID NO: 2)
KYALADASLKMADPNRFRGKDLPVLDQLTDPPGVRRVYHIQAGLPDPFQ
PPSLPITVYYAVLERACRSVLLNAPSEAPQIVRGASEDVRKQPYNLTIA
WFRMGGNCAIPITVMEYTECSYNKSLGACPIRTQPRWNYYDSFSAVSED
NLGFLMHAPAFETAGTYLRLVKINDWTEITQFILEHRAKGSCKYALPLR
IPPSACLSPQAYQQGVTVDSIGMLPRFIPENQRTVAVYSLKIAGWHGPK
APYTSTLLPPELSETPNATQPELAPEDPEDSALLEDPVGTVAPQIPPNW
HIPSIQDAATPYHPPATPNNMGLIAGAVGGSLLAALVICGIVYWMHRRT
RKAPKRIRLPHIREDDQPSSHQPLFY

In one embodiment, the recombinant HSV is derived from oncolytic HSV, in which, the nucleotide sequence encoding amino acids 6-38 of wild type gD (DASLKMADPNRFRGKDLPVLDQLTDPPGVRRVY (SEQ ID NO: 3)) is deleted.

In one embodiment, the recombinant HSV is derived from oncolytic HSV, in which, the nucleotide sequence encoding amino acids 6-38 of wild type gD (SEQ ID NO: 3) is deleted and replaced by a nucleotide sequence encoding a heterologous ligand peptide having a length of 5 to 150 amino acids, or 5 to 120 amino acids, or 5 to 100 amino acids, or 5 to 80 amino acids, or 5 to 60 amino acids, or 5 to 50 amino acids, or 5 to 45 amino acids, or 5 to 40 amino acids, or 10 to 40 amino acids, or 10 to 35 amino acids.

In one embodiment, the recombinant HSV disclosed herein is a GCN4-retargeted recombinant HSV, wherein the heterologous ligand peptide is GCN4 transcription factor or a fragment or epitope thereof. In such GCN4-retargeted recombinant HSV, the nucleotide sequence encoding amino acids 6-38 of wild type gD (SEQ ID NO: 3) is deleted and replaced by a heterologous nucleotide sequence encoding a peptide sequence comprising GCN4 transcription factor or a fragment or epitope thereof. In one aspect, the heterologous nucleotide sequence encodes a peptide sequence comprising a GCN4 epitope (KNYHLENEVARLKKLV, SEQ NO: 4). In another aspect, the heterologous nucleotide sequence encodes a peptide sequence comprising a GCN4-derived peptide (TSGSKNYHLENEVARLKKLVGSGGGGSGNS, SEQ ID NO: 5), which is comprised of the GCN4 epitope (SEQ NO: 4) flanked by linkers.

In one embodiment, the recombinant HSV disclosed herein is a leucine-zipper-retargeted recombinant HSV, wherein the heterologous ligand peptide is a leucine-zipper moiety. In such leucine-zipper-retargeted recombinant HSV, the nucleotide sequence encoding amino acids 6-38 of wild type gD (SEQ ID NO: 3) is deleted and replaced by a heterologous nucleotide sequence encoding a peptide sequence comprising a leucine-zipper moiety (such as those disclosed in Moll J R et al., Designed heterodimerizing leucine zippers with a range of pIs and stabilities up to 10(−15) M. Protein Sci. 2001 March; 10(3):649-55) or a fragment thereof. In one aspect, the recombinant HSV disclosed herein has the nucleotide sequence encoding amino acids 6-38 of wild type gD (SEQ ID NO: 3) deleted and replaced by a nucleotide sequence encoding a peptide sequence comprising the synthetic leucine-zipper moiety RE (LEIRAAFLRQRNTALRTEVAELEQEVQRLENEVSQYETRYGPL, SEQ ID NO: 6; CTGGAAATCAGAGCCGCTTTCCTGAGACAGCGGAACACCGCCCTGCGGACCGA GGTGGCCGAGCTGGAACAGGAGGTGCAGAGACTGGAAAACGAGGTGTCCCAA TACGAGACAAGATACGGCCCTCTG, SEQ ID NO: 7). In a further aspect, the recombinant HSV disclosed herein has the nucleotide sequence encoding amino acids 6-38 of wild type gD (SEQ ID NO: 3) deleted and replaced by a nucleotide sequence encoding a peptide sequence comprising a RE-derived peptide (GTLEIRAAFLRQRNTALRTEVAELEQEVQRLENEVSQYETRYGPLGGGGSGGGGS GGGGSGNS, SEQ ID NO: 8; GGTACCCTGGAAATCAGAGCCGCTTTCCTGAGACAGCGGAACACCGCCCTGCG GACCGAGGTGGCCGAGCTGGAACAGGAGGTGCAGAGACTGGAAAACGAGGTG TCCCAATACGAGACAAGATACGGCCCTCTGGGCGGCGGCGGAAGCGGCGGAG GCGGCAGCGGCGGCGGCGGATCTGGGAATTCT, SEQ ID NO: 9). The RE-derived peptide is comprised of the synthetic leucine-zipper moiety RE (SEQ ID NO: 6) flanked by linkers. In a yet further aspect, the recombinant HSV disclosed herein has the nucleotide sequence encoding amino acids 6-38 of wild type gD (SEQ ID NO: 3) deleted and replaced by a heterologous nucleotide sequence encoding a peptide sequence comprising the synthetic leucine-zipper moiety ER (LEIEAAFLERENTALETRVAELRQRVQRLRNRVSQYRTRYGPL, SEQ ID NO: 10; CTGGAAATCGAGGCCGCCTTCCTGGAACGGGAAAACACCGCCCTGGAGACAA GAGTCGCCGAGCTGAGACAGCGGGTGCAGAGACTGCGGAATAGAGTGTCCCA ATACCGCACCAGATACGGCCCTCTG, SEQ ID NO: 11). In a yet further aspect, the recombinant HSV disclosed herein has the nucleotide sequence encoding amino acids 6-38 of wild type gD (SEQ ID NO: 3) deleted and replaced by a nucleotide sequence encoding peptide sequence comprising a ER-derived peptide, which is comprised of the synthetic leucine-zipper moiety ER (SEQ NO: 10) flanked by linkers.

In one embodiment, the recombinant HSV disclosed herein is a La-retargeted recombinant HSV, wherein the heterologous ligand peptide is La protein or a fragment or epitope thereof. In such La-retargeted recombinant HSVs, the nucleic sequence encoding amino acids 6-38 of wild type gD (SEQ ID NO: 3) is deleted and replaced by a heterologous nucleotide sequence encoding a peptide sequence comprising nuclear autoantigen La protein or a fragment or an epitope thereof (Kohsaka et al, Fine epitope mapping of the human SS-B/La protein. Identification of a distinct autoepitope homologous to a viral gag polyprotein, J Clin Invest. 1990 May; 85(5):1566-74). In one aspect, the recombinant HSV disclosed herein has the nucleotide sequence encoding amino acids 6-38 of wild type gD (SEQ ID NO: 3) deleted and replaced by a heterologous nucleotide sequence encoding a peptide sequence comprising a La epitope (SKPLPEVTDEY, SEQ ID NO: 12) (See e.g., Koristka, S et al, Retargeting of Regulatory T Cells to Surface-inducible Autoantigen La/SS-B, Journal of Autoimmunity 42 (2013) 105-116). In a further aspect, the recombinant HSV disclosed herein has the nucleotide sequence encoding amino acids 6-38 of wild type gD (SEQ ID NO: 3) deleted and replaced by a nucleotide sequence encoding a peptide sequence comprising a La-derived peptide (GTGSKPLPEVTDEYGGGGSGNS, SEQ ID NO: 13; ACCGGCAGCAAGCCCCTGCCCGAGGTGACCGACGAGTACGGCGGCGGCGGCT CCGGGAATTCT, SEQ ID NO: 14), which is comprised of the La epitope (SEQ ID NO: 12) flanked by linkers.

With such modification, the recombinant HSV can be de-targeted from normal cells and, in combination with the bispecific adaptor protein disclosed below, retargeted to diseased cells (e.g., tumor cells).

Specifically, in order for the recombinant HSV disclosed herein be efficiently retargeted to a cell present in cell culture and possibly to a diseased cell, it is advantageous that the binding sites of the recombinant HSV to natural receptors of gD present on normal cells are inactivated. This allows the efficient targeting to cells which are intended to be infected whereas infection of normal cells which are naturally infected by herpesvirus is reduced. gD is essential for virus entry into host cells and plays an essential role in herpesvirus infectivity. The inactivation of binding sites of gD to their natural receptors favors the retargeting to cells carrying the target molecules of the ligand(s). In accordance with the present disclosure, by deleting the nucleotide sequence encoding amino acids 6-38 of gD (SEQ ID NO: 3), both the natural HVEM binding site (amino acids 6-34 of gD (SEQ ID NO: 3)) and the natural nectin-1 binding site (amino acids 35-39 of gD (SEQ ID NO: 3)) of the recombinant HSV are inactivated, such that the binding to cells carrying these receptors is reduced. This results in efficient detargeting of the recombinant HSV from the natural receptors of gD, and, therefore, in the detargeting of the recombinant HSV of the present disclosure from normal cells.

Moreover, the recombinant HSV also is capable of binding to a bispecific adaptor protein (as described below) and can be used, in combination with the bispecific adaptor protein, as effective therapeutics in treating diseases, such as cancer. This embodiment is described in detail below.

Furthermore, the recombinant HSV disclosed herein can be propagated safely. Suitable techniques and conditions for growing HSV in a cell are well known in the art (Florence et al., 1992; Peterson and Goyal, 1988) and include incubating the HSV with the cell and recovering the HSV from the medium of the infected cell culture.

A “cultured” cell is a cell which is present in an in vitro cell culture which is maintained and propagated, as known in the art. Cultured cells are grown under controlled conditions, generally outside of their natural environment. Usually, cultured cells are derived from multicellular eukaryotes, especially animal cells. “A cell line approved for growth of HSV” is meant to include any cell line which has been already shown that it can be infected by a HSV, i.e., the virus enters the cell, and is able to propagate and produce the virus. A cell line is a population of cells descended from a single cell and containing the same genetic composition. In one embodiment, the cells for propagation and production of the recombinant herpesvirus are Vero, 293, 293T, HEp-2, HeLa, BHK, MRC5, or RS cells.

In accordance with the present disclosure, the cell line for propagation and production are modified to carry a target molecule capable of binding to the recombinant HSV disclosed herein. For example, for the recombinant HSVs having the nucleotide sequence encoding all or part of the HVEM binding site and all or part of the nectin-1 binding site is deleted, the cell line for propagation and production may be modified to carry a target molecule (e.g., an antigen binding fragment) having binding specificity to the recombinant HSV. In one particular aspect, the cell lines may be modified to carry an antigen binding fragment having binding specificity to the truncated gD on the recombinant HSV. Or, for the recombinant HSVs having the nucleotide sequence encoding all or part of the HVEM binding site and all or part of the nectin-1 binding site deleted and replaced by a heterologous nucleotide sequence encoding a ligand peptide, the cell line for propagation and production may be modified to carry a target molecule (e.g., an antigen binding fragment) having binding specificity to the ligand peptide.

In one embodiment, the cell line carries a target molecule capable of binding GCN4 transcription factor or a fragment thereof, or an epitope thereof, and can be used to propagate GCN4-retargeted recombinant HSV. In one embodiment, the cell line carries a target molecule, which is an antigen binding fragment or antigen binding domain, capable of binding GCN4 transcription factor or a fragment thereof, or an epitope thereof. In one embodiment, the cell line used herein carries a target molecule, which is an antigen binding fragment, capable of binding a GCN4 epitope identified by SEQ ID NO: 4 or capable of binding a peptide derived from GCN4 epitope, which is identified by SEQ ID NO: 5. In one aspect, the cell line is the Vero cell line which has been modified to express an antigen binding fragment capable of binding GCN4 transcription factor or a fragment thereof, or an epitope thereof. In another aspect, the Vero cell line has been modified to express an antigen binding fragment capable of binding a GCN4 epitope identified by SEQ ID NO: 4 or capable of binding to a peptide derived from GCN4 epitope, which is identified by SEQ ID NO: 5.

In one embodiment, the cell line carries a target molecule capable of binding the leucine-zipper moiety encoded by the recombinant HSV, and can be used to propagate leucine-zipper-retargeted recombinant HSV. In one aspect, the cell line carries a target molecule which is synthetic leucine-zipper moiety ER (SEQ ID NO: 10) or a fragment thereof capable of binding leucine-zipper moiety RE (SEQ ID NO: 6). In a further aspect, the cell line is the Vero cell line which has been modified to express a peptide comprising leucine-zipper moiety ER (SEQ ID NO: 10) or a fragment thereof, which is capable of binding leucine-zipper moiety RE (SEQ ID NO: 6) or a fragment thereof. In a yet further aspect, the cell line carries a target molecule which is synthetic leucine-zipper moiety RE (SEQ ID NO: 6) or a fragment thereof capable of binding leucine-zipper moiety ER (SEQ ID NO: 10). In a yet further aspect, the cell line is the Vero cell line which has been modified to express a peptide comprising leucine-zipper moiety RE (SEQ ID NO: 6) or a fragment thereof, which is capable of binding leucine-zipper moiety ER (SEQ ID NO: 10) or a fragment thereof.

In one embodiment, the cell line carries a target molecule capable of binding La protein or a fragment or epitope thereof, and can be used to propagate La-retargeted recombinant HSV. In one embodiment, the cell line carries a target molecule, which is an antigen binding fragment, capable of binding La protein or a fragment or epitope thereof. In one embodiment, the cell line used herein carries a target molecule, which is an antigen binding fragment, capable of binding a La epitope identified by SEQ ID NO: 12 or capable of binding a peptide derived from La protein, which is identified by SEQ ID NO: 13. In one aspect, the cell line is the Vero cell line which has been modified to express an antigen binding fragment capable of binding La protein or a fragment thereof, or an epitope thereof. In another aspect, the Vero cell line has been modified to express an antigen binding fragment capable of binding a La protein identified by SEQ ID NO: 12 or capable of binding to a peptide derived from La protein, which is identified by SEQ ID NO: 13.

### Bispecific Adaptor Protein

Further disclosed herein are isolated bispecific adaptor proteins, which are engineered to comprise a first binding domain that specifically binds the ligand peptide encoded by the heterologous nucleotide sequence of the recombinant HSV (as described above) and a second binding domain that specifically binds a target, such as, a tumor associated antigen (TAA), or a human TAA.

As disclosed herein, the bispecific adaptor proteins may comprise the first binding domain and the second binding domain linked by a peptide linker. Also within the scope of the present disclosure, the bispecific adaptor proteins may comprise the first and second binding domains conjugated through a intermolecular bond, such as a disulfide bond.

In one embodiment, the ligand peptide is GCN4 transcription factor or a fragment thereof or an epitope thereof. The first binding domain of the bispecific adaptor protein specifically binds GCN4 transcription factor or a fragment thereof, or an epitope of GCN4, or the epitope of GCN4 as identified by SEQ ID NO: 4, or an epitope of GCN4 flanked by linkers as identified by SEQ ID NO: 5.

In one embodiment, the ligand peptide is a leucine-zipper moiety or a fragment thereof, and the first binding domain of the bispecific adaptor protein comprises a pairing leucine zipper moiety specifically binds the ligand peptide. In one aspect, the first binding domain of the bispecific adaptor protein specifically binds leucine-zipper moiety RE or a fragment thereof, or an epitope of leucine-zipper moiety RE, or the leucine-zipper moiety RE as identified by SEQ ID NO: 6, or the leucine-zipper moiety RE flanked by linkers as identified by SEQ ID NO: 8. In yet another embodiment, the first binding domain of the bispecific adaptor protein specifically binds leucine-zipper moiety ER or a fragment thereof, or an epitope of leucine-zipper moiety ER, or the leucine-zipper moiety ER as identified by SEQ ID NO: 10, or the leucine-zipper moiety ER flanked by linkers.

In one embodiment, the ligand peptide is La protein or a fragment thereof or an epitope thereof. The first binding domain of the bispecific adaptor protein specifically binds La protein or a fragment thereof, or an epitope of La, or the epitope of La as identified by SEQ ID NO: 12, or an epitope of La flanked by linkers as identified by SEQ ID NO:13.

As used herein, a binding domain that “specifically binds a ligand peptide or a fragment thereof or an epitope thereof” refers to a binding domain that binds a ligand peptide or a fragment thereof or an epitope thereof, with a KD of 1×10⁻⁷ M or less, or 1×10⁻⁸ M or less, or 5×10⁻⁹ M or less, or 1×10⁻⁹ M or less, or 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD is, the higher affinity the bonding specificity is.

As used herein, the term “tumor associated antigen (TAA)” refers to any antigen expressed and capable of being recognized by an antibody capable of binding the TAA. Examples of TAAs can include, but are not limited to, prostate specific membrane antigen (PSMA), TMEFF2, ROR1, KLK2, HLA-G, CD70, PD-1, PD-L1, CTLA-4, EGFR, HER-2, CD19, CD20, CD3, mesothelin (MSLN), prostate stem cell antigen (PCSA), B-cell maturation antigen (BCMA or BCM), G-protein coupled receptor family C group 5 member D (GPRC5D), Interleukin-1 receptor accessory protein (IL1RAP), delta-like 3 (DLL3), carbonic anhydrase IX (CAIX), carcinoembryonic antigen (CEA), CD5, CD7, CD10, CD22, CD30, CD33, CD34, CD38, CD41, CD44, CD49f, CD56, CD74, CD123, CD133, CD138, epithelial glycoprotein-2 (EGP 2), epithelial glycoprotein-40 (EGP-40), epithelial adhesion molecule (EpCAM), folate-binding protein (FBP), fetal acetylcholine receptor (AChR), folate receptor a and b (FRa and b), ganglioside G2 (GD2), ganglioside G3 (GD3), epidermal growth factor receptor (EGFR), epidermal growth factor receptor VIII (EGFRvIII), ERB3, ERB4, interleukin-13 receptor subunit alpha-2 (IL-13Ra2), k-light chain, kinase insert domain receptor (KDR), Lewis A (CA19.9), Lewis Y (LeY), L1 cell adhesion molecule (LICAM), melanoma-associated antigen 1 (melanoma antigen family A1, MAGE-A1), Mucin-16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor receptor (VEGFR), vascular endothelial growth factor R2 (VEGF-R2), type 1 tyrosine-protein kinase transmembrane receptor (ROR1), B7-H3 (CD276), B7-H6 (Nkp30), chondroitin sulfate proteoglycan-4 (CSPG4), DNAX accessory molecule (DNAM-1), ephrin type A receptor 2 (EpHA2), fibroblast associated protein (FAP), Gp100/HLA-A2, glypican 3 (GPC3), HA-1H, HERK-V, IL-11Ra, latent membrane protein (LMP1), neural cell-adhesion molecule (N-CAM/CD56), and trail receptor (TRAIL R).

As used herein, a binding domain that “specifically binds” or with “binding specificity to” refers to a binding domain that binds a target, with a KD of 1×10⁻⁷ M or less, or 1×10⁻⁸ M or less, or 5×10⁻⁹ M or less, or 1×10⁻⁹ M or less, or 5×10⁻¹⁰ M or less, or 1×10⁻¹⁰ M or less.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies disclosed herein can be of any of the five major classes or corresponding sub-classes. In one embodiment, the antibodies disclosed herein are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies disclosed herein include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds an epitope of the ligand peptide (e.g, GCN4 or La protein) or a TAA is substantially free of antibodies that do not bind the epitope of the ligand peptide or TAA). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “single-chain antibody” refers to a conventional single-chain antibody in the field. One exemplary single-chain antibody is single-chain variable fragment (scFv) comprising a heavy chain variable region and a light chain variable region connected by a short peptide (e.g., a peptide of about 5 to about 20 amino acids). Another exemplary single-chain antibody is single-chain antigen-binding fragment (scFab) comprising one constant and one variable domain of each of the heavy and the light chains. Yet another exemplary single-chain antibody is VHH (or so called nanobody) corresponding to the variable region of a heavy chain of a camelid antibody.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antigen binding domain derived from one species of mammal (e.g., mouse, rat, rabbit, etc.) having the desired specificity, affinity, and capability, while the constant regions correspond to the sequences of an antigen binding domain derived from another species of mammal (e.g., human) to avoid eliciting an immune response in that species.

As used herein, the term “DARPin” (designed ankyrin repeat protein; see Chapter 5. “Designed Ankyrin Repeat Proteins (DARPins): From Research to Therapy”, Methods in Enzymology, vol 503: 101˜134 (2012); and “Efficient Selection of DARPins with Sub-nanomolar Affinities using SRP Phage Display”, J. Mol. Biol. (2008) 382, 1211-1227, the entire disclosures of which are hereby incorporated by reference) refers to an antibody mimetic protein having high specificity and high binding affinity to a target protein, which is prepared via genetic engineering. A DARPin is originated from natural ankyrin protein, and has a structure comprising at least 2 ankyrin repeat motifs, for example, comprising at least 3, 4 or 5 ankyrin repeat motifs. The DARPin can have any suitable molecular weight depending on the number of repeat motifs. For example, the DARPins including 3, 4 or 5 ankyrin repeat motifs may have a molecular weight of about 10 kDa, about 14 kDa, or about 18 kDa, respectively.

DARPin includes a core part that provides structure and a target binding portion that resides outside of the core and binds to a target. The structural core includes a conserved amino acid sequence and the target binding portion includes an amino acid sequence that differs depending on the target.

In one embodiment, the isolated bispecific adaptor protein disclosed herein is an isolated bispecific antibody, wherein each of the first and second binding domains comprises a single-chain antibody, such as scFv, scFab, or VHH.

In a further embodiment, one or both of the first and second binding domains comprises antigen binding fragment, such as DARPin.

In a yet further embodiment, the isolated bispecific adaptor protein comprises, from N-terminus to C-terminus, the first binding domain, a linker (e.g., a (G₄S)ₙ polypeptide linker (n is an integer of at least 2) (SEQ ID NO: 128)) and the second binding domain. Or, the isolated bispecific adaptor protein comprises from N-terminus to C-terminus, the second binding domain, a linker ((G₄S)ₙ polypeptide linker (n is an integer of at least 2) (SEQ ID NO: 128)), and the first binding domain.

In a yet further embodiment, the isolated bispecific adaptor protein may comprise the first binding domain and the second binding domain conjugated through an intermolecular bond, such as a disulfide bond.

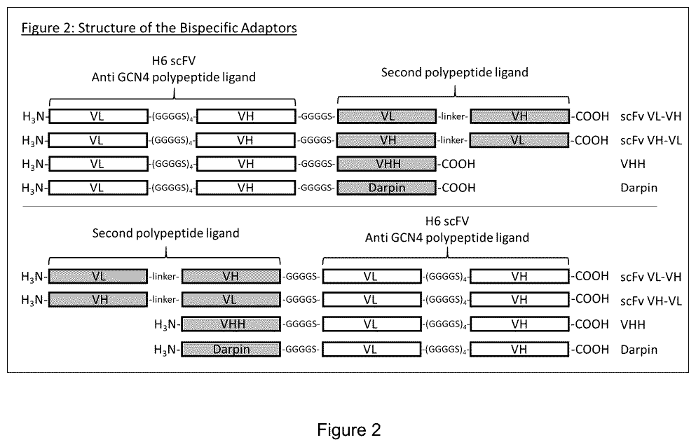

FIG. 2 shows exemplary configurations of bispecific adaptor proteins useful herein. For example, the first binding domain is formed of an anti-GCN4 polypeptide ligand (H6 scFv), which is comprised of, from N-terminus to C-terminus, a light chain variable region (VL) and a heavy chain variable region (HL) linked by a (GGGGS)₄ linker (SEQ ID NO: 15); the second binding domain is formed of single-chain variable fragment scFv, a single-chain antibody VHH, or a polypeptide Darpin having specificity to a target (e.g., tumor cell).

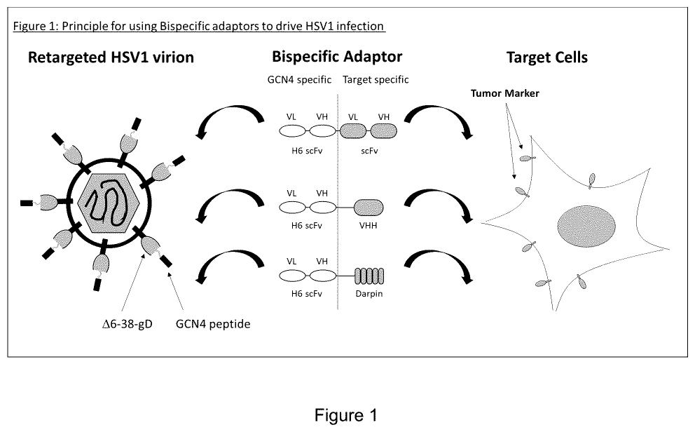

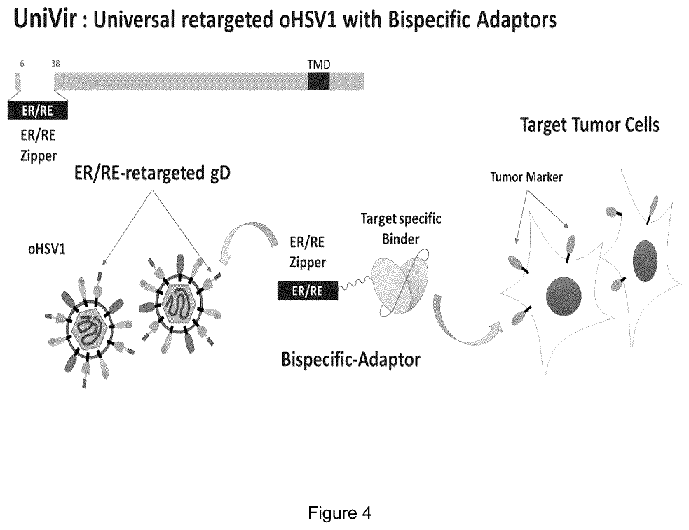

In accordance with the present invention, the bispecific adaptor protein disclosed herein can be used as an adaptor to drive recombinant HSV infection to target cells (such as tumor cells). For example, as shown in FIGS. 1 and 4, with its first binding domain specifically binds the recombinant HSV and a second binding domain specifically binds the target cells (e.g., tumor cells), the bispecific adaptor protein disclosed herein can drive the recombinant HSV virion to the target cells for targeted infection.

### The First Binding Domain

The first binding domain of the bispecific adaptor protein is a ligand-binding domain that specifically binds the ligand peptide encoded by a heterologous nucleotide sequence of the recombinant HSV.

In one embodiment, the first binding domain of the bispecific adaptor protein is a GCN4-binding domain that specifically binds GCN4 transcription factor, or a fragment thereof, or an epitope thereof, or an epitope thereof as identified by SEQ ID NO: 4. The GCN4-binding domain may be an antigen binding fragment. The GCN4-binding domain may comprise a single-chain antibody, such as scFv, scFab, or VHH.

In one embodiment, the GCN4-binding domain comprises a heavy chain variable region (VH) comprising heavy chain complementarity determining region 1 (HCDR1), HCDR2, and HCDR3 and/or a light chain variable region VL comprising light chain complementarity determining region 1 (LCDR1), LCDR2, and LCDR3, the sequences of which are as follows:

HCDR1:
(SEQ ID NO: 16)
GFSLTDYG;
HCDR2:
(SEQ ID NO: 17)
IWGDGIT;
HCDR3:
(SEQ ID NO: 18)
VTGLFDY;
LCDR1:
(SEQ ID NO: 19)
TGAVTTSNY;
LCDR2:
(SEQ ID NO: 20)
GTN;
LCDR3:
(SEQ ID NO: 21)
ALWYSNHWV.

In one aspect, the GCN4-binding domain of the bispecific adaptor protein comprises a VH having a polypeptide sequence at least 95%, or at least 96%, or at least 97%, or at least 98%, or at least 99%, or 100% identical to SEQ ID NO: 22 (DVQLQQSGPGLVAPSQSLSITCTVSGFSLTDYGVNWVRQSPGKGLEWLGVIWGD GITDYNSALKSRLSVTKDNSKSQVFLKMNSLQSGDSARYYCVTGLFDYWGQGTT LTVSS), and/or a VL having a polypeptide sequence at least 95%, or at least 96%, or at least 97%, or at least 98%, or at least 99%, or 100% identical to SEQ ID NO: 23 (DAVVTQESALTTSPGETVTLTCRSSTGAVTTSNYASWVQEKPDHLFTGLIGGTNN RAPGVPARFSGSLIGDKAALTITGAQTEDEAIYFCALWYSNHWVFGGGTKLTVL).

In a further aspect, the GCN4-binding domain of the bispecific adaptor protein is a single chain variable fragment (scFv). The anti-GCN4 scFv may be comprised of a VH domain separated from a VL domain by a (G₄S)ₙ polypeptide linker (n is an integer of at least 2 (SEQ ID NO: 128)). The VH domain has a polypeptide sequence at least 95%, or at least 96%, or at least 97%, or at least 98%, or at least 99%, or 100% identical to SEQ ID NO: 22. The VL domain has a polypeptide sequence at least 95%, or at least 96%, or at least 97%, or at least 98%, or at least 99%, or 100% identical to SEQ ID NO: 23. The anti-GCN4 scFv may be, from N-terminus to C-terminus, in VH-VL orientation or VL-VH orientation. One exemplary anti-GCN4 scFv has, from N-terminus to C-terminus, a VH-VL orientation and a polypeptide sequence at least 95%, or at least 96%, or at least 97%, or at least 98%, or at least 99%, or 100% identical to SEQ ID NO: 24 (DVQLQQSGPGLVAPSQSLSITCTVSGFSLTDYGVNWVRQSPGKGLEWLGVIWGD GITDYNSALKSRLSVTKDNSKSQVFLKMNSLQSGDSARYYCVTGLFDYWGQGTT LTVSSGGGGSGGGGSGGGGSGGGGSDAVVTQESALTTSPGETVTLTCRSSTGAVT TSNYASWVQEKPDHLFTGLIGGTNNRAPGVPARFSGSLIGDKAALTITGAQTEDEA IYFCALWYSNHWVFGGGTKLTVL). Another exemplary anti-GCN4 scFv has, from N-terminus to C-terminus, a VL-VH orientation and a polypeptide sequence at least 95%, or at least 96%, or at least 97%, or at least 98%, or at least 99%, or 100% identical to SEQ ID NO: 25 (DAVVTQESALTTSPGETVTLTCRSSTGAVTTSNYASWVQEKPDHLFTGLIGGTNN RAPGVPARFSGSLIGDKAALTITGAQTEDEAIYFCALWYSNHWVFGGGTKLTVLG GGGSGGGGSGGGGSGGGGSDVQLQQSGPGLVAPSQSLSITCTVSGFSLTDYGVNW VRQSPGKGLEWLGVIWGDGITDYNSALKSRLSVTKDNSKSQVFLKMNSLQSGDS ARYYCVTGLFDYWGQGTTLTVSS) (H6 scFv).

In one embodiment, the first binding domain of the bispecific adaptor protein is a RE-binding domain that specifically binds synthetic leucine-zipper moiety RE (SEQ ID NO: 6) or a fragment thereof. In one aspect, the RE-binding domain comprises an antigen binding fragment capable of binding leucine-zipper moiety RE. In another aspect, the RE-binding domain comprises leucine-zipper moiety ER (SEQ ID NO: 10) or a fragment thereof, which is capable of specifically binds the leucine-zipper moiety RE (SEQ ID NO: 6) or a fragment thereof.

In one embodiment, the first binding domain of the bispecific adaptor protein is an ER-binding domain that specifically binds synthetic leucine-zipper moiety ER (SEQ ID NO: 10) or a fragment thereof. In one aspect, the ER-binding domain comprises an antigen binding fragment capable of binding leucine-zipper moiety ER. In another aspect, the ER-binding domain comprises leucine-zipper moiety RE (SEQ ID NO: 6) or a fragment thereof, which is capable of specifically binds the leucine-zipper moiety ER (SEQ ID NO: 10) or a fragment thereof.

In one embodiment, the first binding domain of the bispecific adaptor protein is a La-binding domain that specifically binds La protein, or a fragment thereof, or an epitope thereof, or an epitope thereof as identified by SEQ ID NO: 12. The La-binding domain may be an antigen binding fragment. The La-binding domain may comprise a single-chain antibody, such as scFv, scFab, or VHH.

In one embodiment, the La-binding domain comprises a VH comprising HCDR1, HCDR2, and HCDR3 and/or a VL comprising LCDR1, LCDR2, and LCDR3, the sequences of which are as follows:

HCDR1:
(SEQ ID NO: 26)
GYTFTHYYIY;
HCDR2:
(SEQ ID NO: 27)
WMGGVNPSNGGTHF;
HCDR3:
(SEQ ID NO: 28)
RSEYDYGLGFAY;
LCDR1:
(SEQ ID NO: 29)
QSLLNSRTPKNYLA;
LCDR2:
(SEQ ID NO: 30)
LLIYWASTRKS;
LCDR3:
(SEQ ID NO: 31)
KQSYNLL.

In one aspect, the La-binding domain of the bispecific adaptor protein comprises a heavy chain variable region (VH) having a polypeptide sequence at least 95%, or at least 96%, or at least 97%, or at least 98%, or at least 99%, or 100% identical to SEQ ID NO: 32 (QVQLVQSGAEVKKPGASVKVSCKASGYTFTHYYIYWVRQAPGQGLEWMGGVN PSNGGTHFNEKFKSRVTMTRDTSISTAYMELSRLRSDDTAVYYCARSEYDYGLGF AYWGQGTLVTVSS), and/or a light chain variable region (VL) having a polypeptide sequence at least 95%, or at least 96%, or at least 97%, or at least 98%, or at least 99%, or 100% identical to SEQ ID NO: 33 (DIVMTQSPDSLAVSLGERATINCKSSQSLLNSRTPKNYLAWYQQKPGQPPKLLIY WASTRKSGVPDRFSGSGSGTDFTLTISSLQAEDVAVYYCKQSYNLLTFGGGTKVEI K)

In a further aspect, the La-binding domain of the bispecific adaptor protein is a single chain variable fragment (scFv). The anti-La scFv may be comprised of a VH domain separated from a VL domain by a (G₄S)ₙ polypeptide linker (n is an integer of at least 2 (SEQ ID NO: 128)). The VH domain has a polypeptide sequence at least 95%, or at least 96%, or at least 97%, or at least 98%, or at least 99%, or 100% identical to SEQ ID NO: 30. The VL domain has a polypeptide sequence at least 95%, or at least 96%, or at least 97%, or at least 98%, or at least 99%, or 100% identical to SEQ ID NO: 31. The anti-La scFv may be, from N-terminus to C-terminus, in VH-VL orientation or VL-VH orientation. One exemplary anti-La scFv has, from N-terminus to C-terminus, a VH-VL orientation and a polypeptide sequence at least 95%, or at least 96%, or at least 97%, or at least 98%, or at least 99%, or 100% identical to SEQ ID NO: 34 (QVQLVQSGAEVKKPGASVKVSCKASGYTFTHYYIYWVRQAPGQGLEWMGGVN PSNGGTHFNEKFKSRVTMTRDTSISTAYMELSRLRSDDTAVYYCARSEYDYGLGF AYWGQGTLVTVSSGGSEGKSSGSGSESKSTGGSDIVMTQSPDSLAVSLGERATINC KSSQSLLNSRTPKNYLAWYQQKPGQPPKLLIYWASTRKSGVPDRFSGSGSGTDFTL TISSLQAEDVAVYYCKQSYNLLTFGGGTKVEIK) (5B9HL).

### The Second Binding Domain

The second binding domain of the bispecific adaptor protein is a TAA-binding domain specifically binds a TAA, such as PSMA, TMEFF2, KLK2, HLA-G, or ROR1. In one aspect, the TAA-binding domain may comprise a single-chain antibody, such as scFv, scFab, or VHH. In another aspect, the TAA-binding domain may comprise an antibody mimetic protein, such as DARPin.

In one embodiment, the second binding domain specifically binds PSMA, such as an anti-PSMA VHH or an anti-PSMA scFv.

In one embodiment, the second binding domain comprises an anti-PSMA VHH. One exemplary anti-PSMA VHH comprises HCDR1 (GSTFSINA, SEQ ID NO: 35), HCDR2 (LSSGGSK, SEQ ID NO: 36), and HCDR3 (NAEIYYSDGVDDGYRGMDY, SEQ ID NO: 37). Or, the exemplary anti-PSMA VHH comprises a polypeptide sequence at least 95%, or at least 96%, or at least 97%, or at least 98%, or at least 99%, or 100% identical to SEQ ID NO: 38 (QLQLVESGGGLVHAGGSLRLSCAASGSTFSINAIGWYRQAPGKQRELVAALSSGG SKNYADSVKGRFTISRDNAKNTVYLQMNRLKPEDTAVYYCNAEIYYSDGVDDGY RGMDYWGKGTQVTVSS (B116)). Another exemplary anti-PSMA VHH comprises HCDR1 (GPPLSSYA, SEQ ID NO: 39), HCDR2 (ISWSGSNT, SEQ ID NO: 40), and HCDR3 (AADRRGGPLSDYEWEDEYAD, SEQ ID NO: 41). Or, the exemplary anti-PSMA VHH comprises a polypeptide sequence at least 95%, or at least 96%, or at least 97%, or at least 98%, or at least 99%, or 100% identical to SEQ ID NO: 42 (EVQVVESGGGLVQTGGSLRLSCAASGPPLSSYAVAWFRQTPGKEREFVAAISWS GSNTYYADSVKGRFTISKDNAKNTVL VYLQMNSLKPEDTAVYYCAADRRGGPLS DYEWEDEYADWGQGTQVTVSS (B110)).

In one embodiment, the second binding domain comprises an anti-PSMA scFv. The anti-PSMA scFv disclosed herein can be, from N-terminus to C-terminus, in VH-VL orientation or VL-VH orientation. In one aspect, the anti-PSMA scFv comprises a VH comprising HCDR1 (GFTFSFYN, SEQ ID NO: 43), HCDR2 (ISTSSSTI, SEQ ID NO: 44), and HCDR3 (AREGSYYDSSGYPYYYYDMDV, SEQ ID NO: 45) and/or a VL comprising LCDR1 (SSNIGAGYD, SEQ ID NO: 46), LCDR2 (GNT, SEQ ID NO: 47), and LCDR3 (QSYDSSLSGTPYVV, SEQ ID NO: 48). In another aspect, the anti-PSMA scFv comprises VH having a polypeptide sequence at least 95%, or at least 96%, or at least 97%, or at least 98%, or at least 99%, or 100% identical to SEQ ID NO: 49 (EVQLVESGGGLVQPGGSLRLSCAASGFTFSFYNMNWVRQAPGKGLEWISYISTSS STIYYADSVKGRFTISRDNAKNSLYLQMNSLRDEDTAVYYCAREGSYYDSSGYPY YYYDMDVWGQGTTVTVSS) and/or VL having a polypeptide sequence at least 95%, or at least 96%, or at least 97%, or at least 98%, or at least 99%, or 100% identical to SEQ ID NO: 50 (QSVLTQPPSVSGAPGQRVTISCTGSSSNIGAGYDVHWYQQLPGTAPKLLIYGNTN RPSGVPDRFSGSKSGTSASLAITGLQAEDEADYYCQSYDSSLSGTPYVVFGGGTKL TVL).

One exemplary anti-PSMA scFv has, from N-terminus to C-terminus, a VH-VL orientation and a polypeptide sequence at least 95%, or at least 96%, or at least 97%, or at least 98%, or at least 99%, or 100% identical to SEQ ID NO: 51 (EVOLVESGGGLVQPGGSLRLSCAASGFTFSFYNMNWVRQAPGKGLEWISYISTSS STIYYADSVKGRFTISRDNAKNSLYLQMNSLRDEDTAVYYCAREGSYYDSSGYPY YYYDMDVWGQGTTVTVSSGGSEGKSSGSGSESKSTGGSQSVLTQPPSVSGAPGQR VTISCTGSSSNIGAGYDVHWYQQLPGTAPKLLIYGNTNRPSGVPDRFSGSKSGTSA SLAITGLQAEDEADYYCQSYDSSLSGTPYVVFGGGTKLTVL (B588HL)). Another exemplary anti-PSMA scFv has, from N-terminus to C-terminus, a VL-VH orientation and a polypeptide sequence at least 95%, or at least 96%, or at least 97%, or at least 98%, or at least 99%, or 100% identical to SEQ ID NO: 52 (QSVLTQPPSVSGAPGQRVTISCTGSSSNIGAGYDVHWYQQLPGTAPKLLIYGNTN RPSGVPDRFSGSKSGTSASLAITGLQAEDEADYYCQSYDSSLSGTPYVVFGGGTKL TVLGGSEGKSSGSGSESKSTGGSEVQLVESGGGLVQPGGSLRLSCAASGFTFSFYN MNWVRQAPGKGLEWISYISTSSSTIYYADSVKGRFTISRDNAKNSLYLQMNSLRD EDTAVYYCAREGSYYDSSGYPYYYYDMDVWGQGTTVTVSS (B588LH)).

In a further embodiment, the second binding domain specifically binds TMEFF2, such as an anti-TMEFF2 scFv. The anti-TMEFF2 scFv disclosed herein may be, from N-terminus to C-terminus, in VH-VL orientation or VL-VH orientation. In one aspect, the anti-TMEFF2 scFv comprises a VH comprising HCDR1 (GFTFSSYS, SEQ ID NO: 53), HCDR2 (ISGSGGFT, SEQ ID NO: 54), and HCDR3 (ARMPLNSPHDY, SEQ ID NO: 55) and/or a VL comprising LCDR1 (QGIRND, SEQ ID NO: 56), LCDR2 (AAS, SEQ ID NO: 57), and LCDR3 (LQDYNYPLT, SEQ ID NO: 58). In one aspect, the anti-TMEFF2 scFv comprises VH having a polypeptide sequence at least 95%, or at least 96%, or at least 97%, or at least 98%, or at least 99%, or 100% identical to SEQ ID NO: 59 (EVQLLESGGGLVQPGGSLRLSCAASGFTFSSYSMSWVRQAPGKGLEWVSVISGSG GFTDYADSVKGRFTISRDNSKNTLYLQMNSLRAEDTAVYYCARMPLNSPHDYWG QGTLVTVSS) and/or VL having a polypeptide sequence at least 95%, or at least 96%, or at least 97%, or at least 98%, or at least 99%, or 100% identical to SEQ ID NO: 60 (DIQMTQSPSSLSASVGDRVTITCRASQGIRNDLGWYQQKPGKAPKLLIYAASSLQS GVPSRFSGSGSGTDFTLTISSLQPEDFATYYCLQDYNYPLTFGGGTKVEIK). In one aspect, the anti-TMEFF2 scFv comprises a VH comprising HCDR1 (GVSISSYF, SEQ ID NO: 61), HCDR2 (ISTSGST, SEQ ID NO: 62), and HCDR3 (VRDWTGFDY, SEQ ID NO: 63) and/or a VL comprising LCDR1 (SSDVGSYNL, SEQ ID NO: 64), LCDR2 (EGS, SEQ ID NO: 65), and LCDR3 (SSYAGSSTYV, SEQ ID NO: 66). In one aspect, the anti-TMEFF2 scFv comprises VH having a polypeptide sequence at least 95%, or at least 96%, or at least 97%, or at least 98%, or at least 99%, or 100% identical to SEQ ID NO: 67 (QVQLQESGPGLVKPSETLSLTCTVSGVSISSYFWSWLRQPAGKGLQWIGRISTSGS TNHNPSLKSRVIMSVDTSKNQFSLKLSSVTAADTAVYYCVRDWTGFDYWGQGTL VTVSS) and/or VL having a polypeptide sequence at least 95%, or at least 96%, or at least 97%, or at least 98%, or at least 99%, or 100% identical to SEQ ID NO: 68 (SYELTQPASVSGSPGQSITISCIGTSSDVGSYNLVSWYQQHPGKVPKLMIYEGSKR PSGVSNRFSGSKSGNTASLTISGLQAEDEADYYCSSYAGSSTYVFGTGTKVTVL).

One exemplary anti-TMEFF2 scFv has, from N-terminus to C-terminus, a VH-VL orientation and a polypeptide sequence at least 95%, or at least 96%, or at least 97%, or at least 98%, or at least 99%, or 100% identical to SEQ ID NO: 69 (QVQLQESGPGLVKPSETLSLTCTVSGVSISSYFWSWLRQPAGKGLQWIGRISTSGS TNHNPSLKSRVIMSVDTSKNQFSLKLSSVTAADTAVYYCVRDWTGFDYWGQGTL VTVSSGGSEGKSSGSGSESKSTGGSSYELTQPASVSGSPGQSITISCIGTSSDVGSYN LVSWYQQHPGKVPKLMIYEGSKRPSGVSNRFSGSKSGNTASLTISGLQAEDEADY YCSSYAGSSTYVFGTGTKVTVL (TMEF9HL)). Another exemplary anti-TMEFF2 scFv has, from N-terminus to C-terminus, a VL-VH orientation and a polypeptide sequence at least 95%, or at least 96%, or at least 97%, or at least 98%, or at least 99%, or 100% identical to SEQ ID NO: 70 (DIQMTQSPSSLSASVGDRVTITCRASQGIRNDLGWYQQKPGKAPKLLIYAASSLQS GVPSRFSGSGSGTDFTLTISSLQPEDFATYYCLQDYNYPLTFGGGTKVEIKGGSEGK SSGSGSESKSTGGSEVQLLESGGGLVQPGGSLRLSCAASGFTFSSYSMSWVRQAPG KGLEWVSVISGSGGFTDYADSVKGRFTISRDNSKNTLYLQMNSLRAEDTAVYYCA RMPLNSPHDYWGQGTLVTVSS (TMEF847LH)). Yet another exemplary anti-TMEFF2 scFv has, from N-terminus to C-terminus, a VL-VH orientation and a polypeptide sequence at least 95%, or at least 96%, or at least 97%, or at least 98%, or at least 99%, or 100% identical to SEQ ID NO: 71 (SYELTQPASVSGSPGQSITISCIGTSSDVGSYNLVSWYQQHPGKVPKLMIYEGSKR PSGVSNRFSGSKSGNTASLTISGLQAEDEADYYCSSYAGSSTYVFGTGTKVTVLGG SEGKSSGSGSESKSTGGSQVQLQESGPGLVKPSETLSLTCTVSGVSISSYFWSWLRQ PAGKGLQWIGRISTSGSTNHNPSLKSRVIMSVDTSKNQFSLKLSSVTAADTAVYYC VRDWTGFDYWGQGTLVTVSS (TMEF9LH)).

In a yet further embodiment, the second binding domain specifically binds KLK2, such as an anti-KLK2 scFv. The anti-KLK2 scFv disclosed herein may be, from N-terminus to C-terminus, in VH-VL orientation or VL-VH orientation. In one aspect, the anti-KLK2 scFv comprises HCDR1 (GNSITSDYA, SEQ ID NO: 72), HCDR2 (ISYSGST, SEQ ID NO: 73), HCDR3 (ATGYYYGSGF, SEQ ID NO: 74), LCDR1 (ESVEYFGTSL, SEQ ID NO: 75), LCDR2 (AAS, SEQ ID NO: 76), and LCDR3 (QQTRKVPYT, SEQ ID NO: 77). In another aspect, the anti-KLK2 scFv comprises VH having a polypeptide sequence at least 95%, or at least 96%, or at least 97%, or at least 98%, or at least 99%, or 100% identical to SEQ ID NO: 78 (QVQLQESGPGLVKPSDTLSLTCAVSGNSITSDYAWNWIRQPPGKGLEWIGYISYS GSTTYNPSLKSRVTMSRDTSKNQFSLKLSSVTAVDTAVYYCATGYYYGSGFWGQ GTLVTVSS) and/or VL having a polypeptide sequence at least 95%, or at least 96%, or at least 97%, or at least 98%, or at least 99%, or 100% identical to SEQ ID NO: 79 (DIVLTQSPDSLAVSLGERATINCKASESVEYFGTSLMHWYQQKPGQPPKLLIYAAS NRESGVPDRFSGSGSGTDFTLTISSLQAEDVAVYYCQQTRKVPYTFGQGTK). In yet another aspect, the anti-KLK2 scFv comprises VH having a polypeptide sequence at least 95%, or at least 96%, or at least 97%, or at least 98%, or at least 99%, or 100% identical to SEQ ID NO: 80 (QVQLQESGPGLVKPSQTLSLTCTVSGNSITSDYAWNWIRQFPGKRLEWIGYISYSG STTYNPSLKSRVTISRDTSKNQFSLKLSSVTAADTAVYYCATGYYYGSGFWGQGT LVTVSS) and/or VL having a polypeptide sequence at least 95%, or at least 96%, or at least 97%, or at least 98%, or at least 99%, or 100% identical to SEQ ID NO: 81 (EIVLTQSPATLSLSPGERATLSCRASESVEYFGTSLMHWYQQKPGQPPRLLIYAAS NVESGIPARFSGSGSGTDFTLTISSVEPEDFAVYFCQQTRKVPYTFGGGTKVEIK).

One exemplary anti-KLK2 scFv has, from N-terminus to C-terminus, a VH-VL orientation and a polypeptide sequence at least 95%, or at least 96%, or at least 97%, or at least 98%, or at least 99%, or 100% identical to SEQ ID NO: 82 (QVQLQESGPGLVKPSDTLSLTCAVSGNSITSDYAWNWIRQPPGKGLEWIGYISYS GSTTYNPSLKSRVTMSRDTSKNQFSLKLSSVTAVDTAVYYCATGYYYGSGFWGQ GTLVTVSSGTEGKSSGSGSESKSTDIVLTQSPDSLAVSLGERATINCKASESVEYFG TSLMHWYQQKPGQPPKLLIYAASNRESGVPDRFSGSGSGTDFTLTISSLQAEDVAV YYCQQTRKVPYTFGQGTKLEIK (11B6HL)). Another exemplary anti-KLK2 scFv has, from N-terminus to C-terminus, a VH-VL orientation and a polypeptide sequence at least 95%, or at least 96%, or at least 97%, or at least 98%, or at least 99%, or 100% identical to SEQ ID NO: 83 (QVQLQESGPGLVKPSQTLSLTCTVSGNSITSDYAWNWIRQFPGKRLEWIGYISYSG STTYNPSLKSRVTISRDTSKNQFSLKLSSVTAADTAVYYCATGYYYGSGFWGQGT LVTVSSGGSEGKSSGSGSESKSTGGSEIVLTQSPATLSLSPGERATLSCRASESVEYF GTSLMHWYQQKPGQPPRLLIYAASNVESGIPARFSGSGSGTDFTLTISSVEPEDFAV YFCQQTRKVPYTFGGGTKVEIK (KL2B359HL)). Yet exemplary anti-KLK2 scFv has, from N-terminus to C-terminus, a VL-VH orientation and a polypeptide sequence at least 95%, or at least 96%, or at least 97%, or at least 98%, or at least 99%, or 100% identical to SEQ ID NO: 84 (DIVLTQSPDSLAVSLGERATINCKASESVEYFGTSLMHWYQQKPGQPPKLLIYAAS NRESGVPDRFSGSGSGTDFTLTISSLQAEDVAVYYCQQTRKVPYTFGQGTKLEIKG TEGKSSGSGSESKSTQVQLQESGPGLVKPSDTLSLTCAVSGNSITSDYAWNWIRQP PGKGLEWIGYISYSGSTTYNPSLKSRVTMSRDTSKNQFSLKLSSVTAVDTAVYYCA TGYYYGSGFWGQGTLVTVSS (11B6LH)). Yet exemplary anti-KLK2 scFv has, from N-terminus to C-terminus, a VL-VH orientation and a polypeptide sequence at least 95%, or at least 96%, or at least 97%, or at least 98%, or at least 99%, or 100% identical to SEQ ID NO: 85 (EIVLTQSPATLSLSPGERATLSCRASESVEYFGTSLMHWYQQKPGQPPRLLIYAAS NVESGIPARFSGSGSGTDFTLTISSVEPEDFAVYFCQQTRKVPYTFGGGTKVEIKGG SEGKSSGSGSESKSTGGSQVQLQESGPGLVKPSQTLSLTCTVSGNSITSDYAWNWI RQFPGKRLEWIGYISYSGSTTYNPSLKSRVTISRDTSKNQFSLKLSSVTAADTAVYY CATGYYYGSGFWGQGTLVTVSS (KL2B359LH)).

In a yet further embodiment, the second binding domain specifically binds HLA-G, such as an anti-HLA-G scFv. The anti-HLA-G scFv disclosed herein may be, from N-terminus to C-terminus, in VH-VL orientation or VL-VH orientation.

In a yet further embodiment, the second binding domain specifically binds ROR1 such as a polypeptide ligand, DARPin. An exemplary DARPin having a specificity for ROR1 has a polypeptide sequence at 95%, or at least 96%, or at least 97%, or at least 98%, or at least 99%, or 100% identical to SEQ ID NO: 86 (GSDLGKKLLEAARAGQDDEVRILMANGADVNASDRYGRTPLHLAAFNGHLEIVE VLLKNGADVNAKDKIGNTPLHLAANHGHLEIVEVLLKYGAVVNATDWLGVTPLH LAAVFGHLEIVEVLLKYGADVNAQDKFGKTAFDISIDNGNEDLAEILQKL (H6w, see e.g., Koch, Characterisation and affinity maturation of DARPins binding human ROR1, Master's Thesis, Submitted at Department of Biotechnology, University of Natural Resources and Life Sciences, Vienna)).

In a yet further embodiment, the invention relates to an isolated polynucleotide comprising a nucleic acid encoding the bispecific adaptor protein or fragment thereof. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding the bispecific adaptor protein or fragment thereof of the invention can be altered without changing the amino acid sequences of the proteins.

In a yet further embodiment of the present disclosure, the invention relates to a vector comprising an isolated polynucleotide comprising the nucleic acid encoding the bispecific adaptor protein or fragment thereof as disclosed herein. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antigen binding domain thereof in the cell. Conventional cloning techniques or artificial gene synthesis can be used to generate a recombinant expression vector according to embodiments of the invention.

In a yet further embodiment, the invention relates to a cell transduced with the vector comprising the isolated polynucleotide comprising a nucleic acid encoding the bispecific adaptor protein or fragment thereof as disclosed herein. The term “transduced” or “transduction” refers to a process by which exogenous nucleic acid is transferred or introduced into the host cell. A “transduced” cell is one which has been transduced with exogenous nucleic acid. The cell includes the primary subject cell and its progeny.

In another general aspect, the invention relates to a method of preparing a transformed cell by transducing a cell with a vector comprising the isolated nucleic acids encoding the bispecific adaptor protein or fragment thereof as disclosed herein.

In another general aspect, the invention relates to a host cell comprising an isolated nucleic acid encoding the bispecific adaptor protein or fragment thereof as disclosed herein. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In a yet further embodiment of the disclosure, the invention relates to a method of producing an isolated bispecific adaptor protein as disclosed herein, comprising culturing a cell comprising a nucleic acid encoding the bispecific adaptor protein as disclosed herein and recovering the bispecific adaptor protein from the cell or cell culture (e.g., from the supernatant). Expressed bispecific adaptor protein can be harvested from the cells and purified according to conventional techniques known in the art and as described herein.

### Pharmaceutical Compositions

Yet further disclosed herein is a pharmaceutical composition comprising a recombinant HSV as disclosed above, an isolated bispecific adaptor protein as disclosed above, and a pharmaceutically acceptable carrier. The term “pharmaceutical composition” as used herein means a product comprising a recombinant HSV as disclosed above, an isolated bispecific adaptor protein as disclosed above, together with one or more pharmaceutically acceptable carriers.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, host cell, virus, and/or engineered immune cell pharmaceutical composition can be used in the invention.

### Methods of Use

In another general aspect, the invention relates to a method of retargeting the recombinant HSV disclosed above to a tumor cell using the bispecific adaptor protein disclosed above. The method comprising administering the recombinant HSV and the bispecific adaptor protein to a subject, wherein, the first binding domain of the bispecific adaptor protein specifically binds the recombinant HSV, the second binding domain of the bispecific adaptor protein specifically binds a TAA of the tumor cell, and thereby recombinant HSV is retargeted to the tumor cell.

In this method, the recombinant HSV and the bispecific adaptor protein are chosen such that the first domain of the bispecific adaptor protein specifically binds the heterologous ligand peptide expressed by the recombinant HSV and the second domain of the bispecific adaptor protein specifically binds a TAA on the surface of a chosen tumor cell. For example, to retarget a recombinant HSV to prostate cancer cell, one may choose a GCN4-retargeted recombinant HSV and a bispecific adaptor protein having a first binding domain comprising an anti-GCN4 scFv and a second binding domain comprising an anti-PSMA scFv.

In another general aspect, the invention relates to a method of treating a cancer in a subject in need thereof, comprising administering to the subject pharmaceutical compositions comprising the recombinant HSV with the matching bispecific adaptor protein as disclosed herein. By this method, the recombinant HSV is retargeted to the cancer cells in a subject by the matching bispecific adaptor protein, and thereby causing oncolysis of the cancer cells. As used herein, “oncolysis” refers to a decrease of viability of the target cancer cells. The viability can be determined by a viable cell count of the treated cells, and the extent of decrease can be determined by comparing the number of viable cells in the treated cells to that in the untreated cells, or by comparing the viable cell count before and after the treatment.

The cancer can, for example, be selected from but not limited to, a prostate cancer, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the pharmaceutical compositions comprising the recombinant HSV and the bispecific adaptor protein comprises a therapeutically effective amount of the recombinant HSV and the bispecific adaptor protein as disclosed herein. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to the recombinant HSV and the bispecific adaptor proteins, a therapeutically effective amount means an amount of the recombinant HSV in combination with the bispecific adaptor protein that modulates an immune response in a subject in need thereof. Also, as used herein with reference to the recombinant HSV, a therapeutically effective amount means an amount of the recombinant HSV with the bispecific adaptor protein that results in treatment of a disease, disorder, or condition; prevents or slows the progression of the disease, disorder, or condition; or reduces or completely alleviates symptoms associated with the disease, disorder, or condition.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the pharmaceutical compositions described herein are formulated to be suitable for the intended route of administration to a subject. For example, the pharmaceutical compositions described herein can be formulated to be suitable for intravenous, subcutaneous, or intramuscular administration.

The pharmaceutical compositions of the invention can be administered in any convenient manner known to those skilled in the art. For example, the pharmaceutical compositions of the invention can be administered to the subject by aerosol inhalation, injection, ingestion, transfusion, implantation, and/or transplantation. The pharmaceutical compositions comprising the recombinant HSVs and the matching bispecific adaptor proteins of the invention can be administered transarterially, subcutaneously, intradermaly, intratumorally, intranodally, intramedullary, intramuscularly, intrapleurally, by intravenous (i.v.) injection, or intraperitoneally. In certain embodiments, the pharmaceutical compositions of the invention can be administered with or without lymphodepletion of the subject.

The pharmaceutical compositions comprising the recombinant HSV and the bispecific adaptor proteins as disclosed herein can be provided in sterile liquid preparations, typically isotonic aqueous solutions with cell suspensions, or optionally as emulsions, dispersions, or the like, which are typically buffered to a selected pH. The pharmaceutical compositions can comprise carriers, for example, water, saline, phosphate buffered saline, and the like, suitable for the integrity and viability of the recombinant HSVs and the bispecific adaptor proteins, and for administration of the pharmaceutical compositions.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are pharmaceutical compositions comprising the recombinant HSVs and the matching bispecific adaptor proteins used in the treatment of a cancer. For cancer therapy, the provided pharmaceutical compositions can be used in combination with another treatment including, but not limited to, a chemotherapy, an anti-CD20 mAb, an anti-TIM-3 mAb, an anti-LAG-3 mAb, an anti-EGFR mAb, an anti-HER-2 mAb, an anti-CD19 mAb, an anti-CD33 mAb, an anti-CD47 mAb, an anti-CD73 mAb, an anti-DLL-3 mAb, an anti-apelin mAb, an anti-TIP-1 mAb, an anti-FOLR1 mAb, an anti-CTLA-4 mAb, an anti-PD-L1 mAb, an anti-PD-1 mAb, other immuno-oncology drugs, an antiangiogenic agent, a radiation therapy, an antibody-drug conjugate (ADC), a targeted therapy, or other anticancer drugs.

According to particular embodiments, the methods of treating cancer in a subject in need thereof comprise administering to the subject the recombinant HSV in combination with the bispecific adaptor protein as disclosed herein.

### Kits

In another general aspect, provided herein are kits, unit dosages, and articles of manufacture comprising the recombinant HSV as disclosed herein, the isolated bispecific adaptor protein as disclosed herein, and optionally a pharmaceutical carrier. In certain embodiments, the kit provides instructions for its use.

In another particular aspect, provided herein are kits comprising (1) a recombinant HSV as disclosed herein and (2) an isolated bispecific adaptor protein or fragment thereof as disclosed herein. The recombinant HSV and the isolated bispecific adaptor protein may be included in the kits as separate component or as a pre-mix.

In another particular aspect, provided herein are kits comprising (1) a recombinant HSV as disclosed herein and (2) an isolated nucleic acid encoding a bispecific adaptor protein or fragment thereof as disclosed herein. The recombinant HSV and the isolated nucleic acid may be included in the kits as separate component or as a pre-mix.

### Examples

HSV Retargeting by GCN4/H6 scFv

### Material & Methods

### Cell Culture

Vero cells (Vero ATCC CCL-81) were maintained in Dulbecco's Modification of Eagle's Medium (DMEM) supplemented with 4.5 g/L glucose, sodium pyruvate, Glutamax (Gibco) and Penicillin/Streptomycin (Lonza, 100 U/mL). Serum-free Vero (VERO-SF-ACF MCB from BioReliance cGMP Biomaterial Repository) were maintained is VP-SFM (ThermoFisher) supplemented with Glutamax (Gibco) and Penicillin/Streptomycin (Lonza, 100U/mL). HEK293T were maintained in Dulbecco's Modification of Eagle's Medium (DMEM) supplemented with 4.5 g/L glucose, sodium pyruvate, Glutamax (Gibco) and Penicillin/Streptomycin (Lonza, 100 U/mL). 22Rv1 cells were maintained in Roswell Park Memorial Institute 1460 Medium (RPMI-1460) supplemented with 4.5 g/L glucose, sodium pyruvate, Glutamax (Gibco) and Penicillin/Streptomycin (Lonza, 100U/mL). LNCaP were maintained in Dulbecco's Modification of Eagle's Medium (DMEM) without phenol red, supplemented with 4.5 g/L glucose, sodium pyruvate, Glutamax (Gibco) and Penicillin/Streptomycin (Lonza, 100U/mL). DU145 were maintained in Eagle's Minimal Essential Medium (EMEM) with EBSS and 25 mM Hepes supplemented with MEM Nonessential Amino Acids (Corning Cellgro), sodium pyruvate, Glutamax (Gibco) and Penicillin/Streptomycin (Lonza, 100U/mL).

GCN4-Retargeted oHISV1 Bacterial Artificial Chromosome (BAC)

The GCN4-retargeted HSV1 BAC (or recombinant HSV1) contains the HSV1 Patton strain genome (see e.g., Mulvey et al., J Virol. 2007 April; 81(7):3377-90 for full description) into which an EGFP-FRT-KAN-FRT-T2A-1XGCN-d6-38gD cassette was inserted between the start codon and the stop codon of the US6 gene (genebank MF959544.1 nucleotide 138309 to 139493). The cassette contains an in frame fusion between the enhanced Green Fluorescent Protein (EGFP) amino acid sequence (Uniprot P42212, F64L and S65T mutations), a peptide linker (AA sequence:

(SEQ ID NO: 129)
SGLEQLESIINFEKLTEWTSHMGSSYSLESIGTSHM),

containing an OVA peptide (underlined) and an in frame FRT site (italic bold, nucleotide sequence gaagttcctattctctagaaagtataggaacttc) (SEQ ID NO: 130), a T2A self-cleaving peptide (AA sequence: GSGEGRGSLLTCGDVEENPGP) (SEQ ID NO: 131), the US6 amino acids 1 to 30 containing the endogenous US6 signal peptide (AA sequence

(SEQ ID NO: 132)
MGGAAARLGAVILFVVIVGLHGVRGKYALA,

signal peptide is underlined), a 30 AA insertion containing the GCN4 epitope peptide (sequence

(SEQ ID NO: 5)
TSGSKNYHLENEVARLKKLVGSGGGGSGNS,

epitope underlines (SEQ ID NO: 4)) and US6 AA 39-369 (Uniprot P57083).

### GCN4-Retargeted HSV1

The GCN4-retargeted virus was obtained by transfection of 1e6 cells of the gD complementing VSF cell line eF9 with 1 μg of GCN4-retargeted HSV1 BAC with lipofectamine 3000. The virus was subsequently amplified by passaging on Vero H6-nectin1 cell.

### gD Complementing VSF Cell Line

Serum-Free Vero cells (VERO-SF-ACF MCB from BioReliance cGMP Biomaterial Repository) were transduced with a lentivirus carrying a 5.7 kb fragment of the HSV1 Patton strain genome containing an EGFP-T2A-US6 (glycoprotein D) cassette inserted in place of the endogenous US6 gene. The EGFP-T2A-US6 ORF is flanked by 1.5 kb of genomic sequences upstream of US6 ORF and 2.2 kb of genomic sequences downstream of the US6 ORF. After selection with blasticidin (2 ug/mL), single cell clones were isolated by limit dilution. Clones were screened for their ability to rescue the growth of a gD deficient HSV1 BAC clone.

H6-nectin1 Cell Lines

Vero cells (ATCC CCL-81) and B16-F10 cells (ATCC, cat no. CRL-6475TM) were transduced with a lentivirus expressing the anti-GCN4 H6 scFv fused to the AA 146-517 of human Nectin-1 (Uniprot Q15223) separated by a G₄S linker (SEQ ID NO: 124). After blasticidin selection (7.5 μg/mL and 10 μg/mL respectively), single cell clones were isolated by limit dilution and screened for H6-nectin1 expression by western blot.

### PSMA Cell Lines

HEK-293T were transduced with a lentivirus expressing the human PSMA (Genecopoeia, Catalog #: LPP-G0050-Lv105-050-S). After puromycin selection (2.5 μg/mL), single cell clones were isolated by limit dilution and screened for PSMA expression by western blot and FACS analysis.

### TMEFF2 Cell Line

Vero cells (ATCC CCL-81) were transduced with a lentivirus expressing human TMEFF2. After puromycin selection (5 μg/mL), a stable population was enriched for PSMA expression by cell sorting.

KLK2-nectin1 Cell Line

Vero cells (ATCC CCL-81) were transduced with a lentivirus expressing human KLK2 (AA 25-261, uniport P20151) bearing the S195A mutation (catalytic dead mutant) fused to the AA 337-517 of human Nectin-1 (Uniprot Q15223, transmembrane+cytoplasmic domains). After puromycin selection (5 μg/mL), a stable population was enriched for KLK2-nectin1 expression by cell sorting.

### Transfection and Expression of Bispecific Adaptor Proteins

All bispecific adaptor proteins used in this study (see Table 1) were cloned into the pCDNA3.1(+)-myc-HisA vector (ThermoFischer).

For transfection, HEK293T cells were seeded in 24 wells in complete DMEM. 24 hour after seeding, cells were transfected with 500 ng of each bispecific adaptor expression plasmid using lipofectamine 3000 (ThermoFischer) according to manufacturer's instructions. 48 hour post transfection, the supernatants were harvested and used immediately for GCN4-retargeted HSV1 Infection Assay.

### GCN4-Retargeted HSV1 Infection Assay

Target cells were seeded in 96 well plates treated with poly-L lysine (Sigma, 0.01%, 30 min at RT, washed twice with DPBS) 24 hr prior to infection. On the day of infection, medium was removed and replaced by 50 uL of conditioned supernatants containing the bispecific adaptor proteins. One untreated well was trypsinized and cells counted. After 2 hr incubation at 37° C., the conditioned medium was removed, cells were washed with 100 uL PBS (except HEK293T cells) and 50 uL of fresh complete medium containing the retargeted virus diluted at MOI=0.1 is added. Cells were incubated at 37° C. for 3 Hr. Viral supernatants were removed, wells were washed with 100 uL PBS (except HEK293T cells) and 100 uL of fresh complete medium was added. After 24 hr, GFP fluorescence and cytopathic effect were monitored by microscopy.

### Western Blot

75 μL of supernatant were mixed with 25 uL 4× Laemmli buffer (Biorad+100 mM DTT) and denatured 5 min at 95° C. 20 uL of each denatured supernatant were run on a 4-15% Mini-PROTEAN® TGX Stain-Free™ Protein Gel (Biorad) and transferred to a low fluorescent PVDF membrane (Biorad, Trans-Blot Turbo Transfer System RTA Transfer kit). Intercept (PBS) blocking buffer (Li-CoR) was used as a blocking buffer. Myc-tagged Bispecific adaptors were detected with c-Myc mouse Monoclonal Antibody (9E10, Invitrogen) as a primary antibody and IRDye 800 CW Goat anti-mouse (Licor) as a secondary antibody. Blots were scanned with Odyssey CLX scanner (Licor).

### FACS Staining

Stable cell lines and their parental counterparts were stained with the following antibodies: PE-labeled anti-ROR1 (Biolegend, 357803), JF646 labeled Anti-TMEFF2 (J4B6, NOVUSBIO), PE labeled anti-PSMA antibody (abcam, ab77228), PE labeled mouse IgG1, K Isotype Ctrl (eBioscience), PE labeled anti-DYDDDDK (SEQ ID NO: 133) (Biolegend). Briefly, 1e6 cells were used per staining in a 100 uL volume. After washing in PBS, cells were stained according to the antibody manufacturer's specifications in PBS+0.5% BSA (SigmaAldrich) for 30 min at 4° C. After washing in PBS, the cells were fixed with 4% PFA (Alfa Aesar) in PBS. Samples were analyzed on a MACSQuant Analyzer 10 (Miltenyi Biotec).

### In Vitro Fusion Assay

In this assay, dual split protein (DSP) reporter (see e.g., Kondo N, Miyauchi K, Meng F, Iwamoto A, Matsuda Z. Conformational changes of the HIV-1 envelope protein during membrane fusion are inhibited by the replacement of its membrane-spanning domain. J Biol Chem. 2010 May 7; 285(19): 14681-8) was used. For the seeding of the effector cells, HEK293T cells were split ⅙ into a 96-well clear bottom/white wall plate. For the seeding of the target cells, HEK293T or HEK293T-PSMA were split ¼ into 12-well plates. The next day, effector cells in 96-well were each transfected using lipofectamine 3000 (ThermoFischer) in OptiMEM with a mixture of 180 ng plasmids expressing HSV1 glycoproteins gB, gH, gL and gD (or the corresponding gD fusion) as well as the split-protein reporter cDSP in a 1:2:2:1:3 mass ratio. The target cells in 12-well were transfected similarly with 1 μg of a 1:1:1 mixture plasmids expressing the corresponding target proteins (except for 293T-PSMA receiving the same amount of an empty vector), the corresponding adaptors (control samples receive the same amount of an empty expression vector) and the split-protein reporter nDSP. The next day, culture medium in the 96-well plate was replaced with Phenol red-free culture medium containing 60 μM Enduren (live cell-permeable luciferase substrate, Promega), target cells were detached with versene solution (Gibco), washed in with phenol red free culture medium, resuspended in phenol red-free culture medium containing 60 μM Enduren and added to the effector cells. Seven hours after addition of the target cells to the effector cells, luciferase activity resulting from cell fusion is measured using a cytation5 multimode plate reader (Biotek) in luminometer mode.

### Results

Oncolytic HSV1 (oHSV1) was retargeted by replacing the amino acids 6-38 of gD (SEQ ID NO: 3) by a 30 AA peptide (SEQ ID NO: 5) containing a 16 AA epitope (SEQ ID NO: 4) from the GCN4 yeast transcription factor for which a picomolar affinity single chain antibody fragment (H6 scFv, referred to as H6 herein) was available (see, e.g., Zahnd et al., J Biol Chem. 2004 Apr. 30; 279(18):18870-7). The resulting polypeptide is also referred to as 1XGCN-d6-38-gD herein. The genetic modification was obtained by recombination at the endogenous glycoprotein D locus between the oHSV1 genome in a bacterial artificial chromosome (1) and an expression cassette containing the Enhanced Green Fluorescent Protein (EGFP) sequence separated from 1XGCN-d6-38-gD by a T2A self-cleaving peptide (see material and methods). The resulting virus hence uses the 5′ and 3′ UTRs of the endogenous US6 locus to control the expression of the EGFP-T2A-1XGCN-d6-38-gD cassette leading to expression of the retargeted 1XGCN-d6-38-gD at the virus surface and of EGFP in the infected cells.

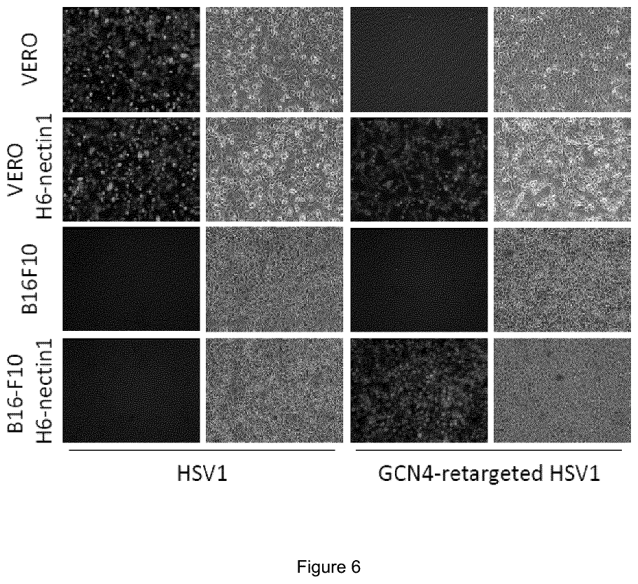

The GCN4/H6 retargeting and the specificity of the virus were first tested by infecting B16-F10 and Vero cell lines stably expressing an H6-nectin1 fusion protein at their surface. As shown in FIG. 6, the GCN4/H6 retargeted virus could infect both Vero and B16-F10 cell lines expressing H6-nectin1 but was unable to infect their parental counterparts. Conversely, an oHSV1 expressing the wild-type gD glycoprotein could infect the parental Vero cell line that expresses nectin-1 at its surface but was unable to infect the B16-F10 parental line that lacks nectin-1 expression. Altogether these results confirmed the GCN4-retargeted virus had lost its ability to infect cells using nectin1 as a receptor but was able to use the H6-nectin1 fusion as its receptor for cell entry.

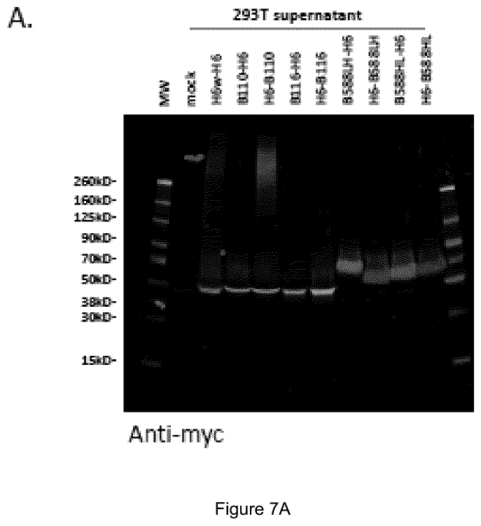

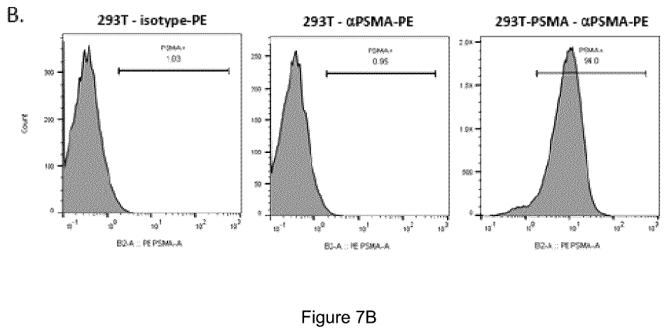

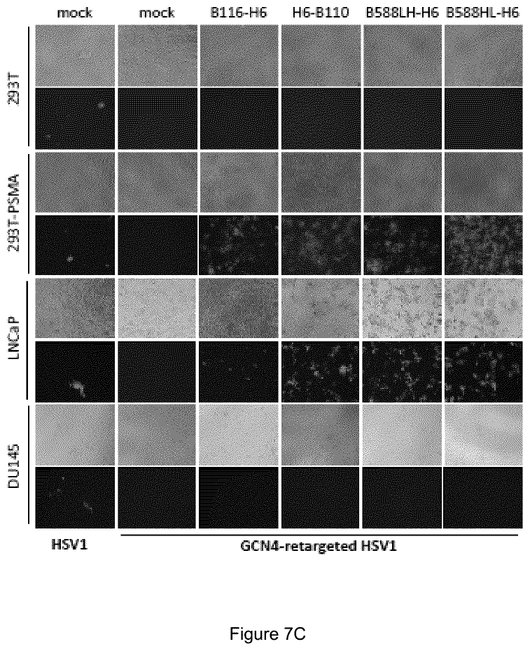

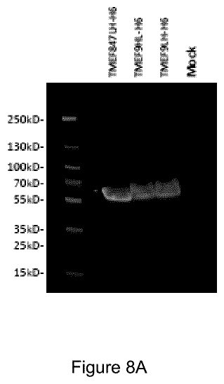

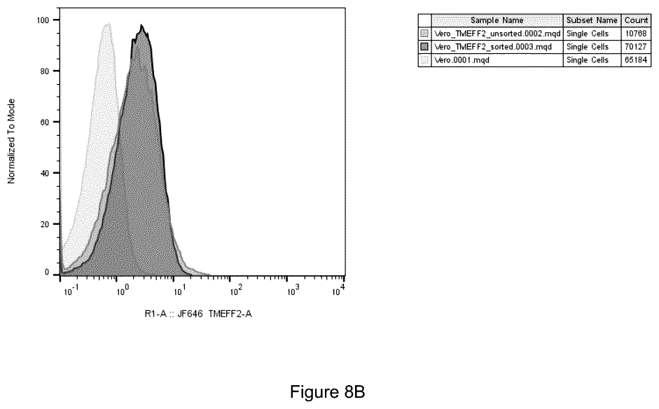

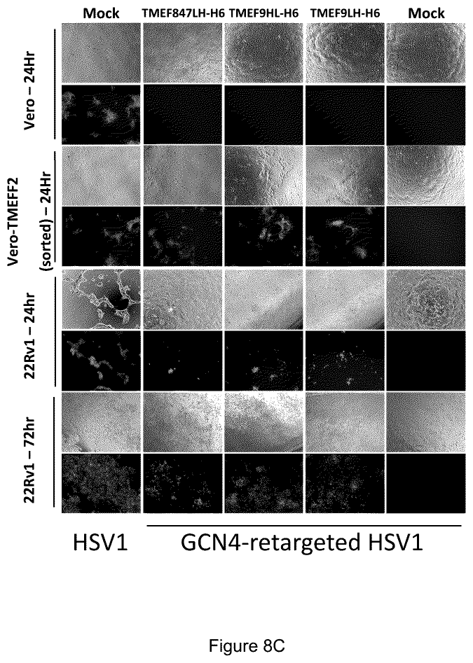

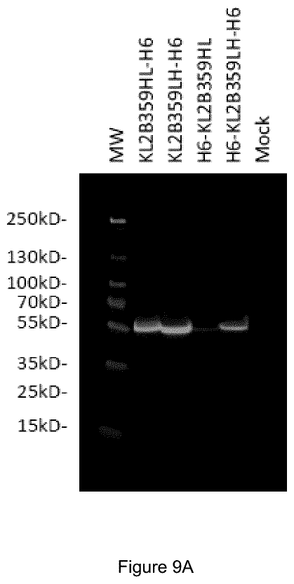

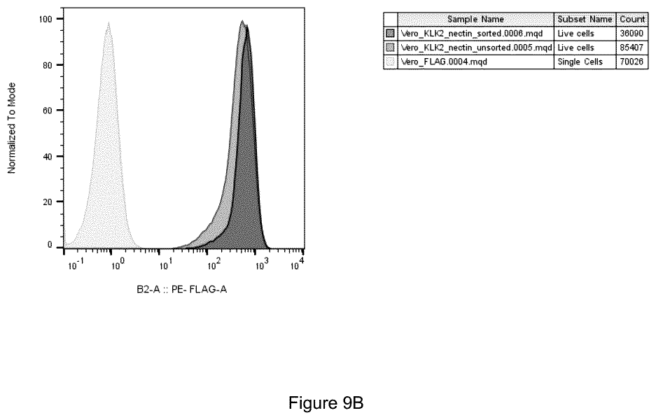

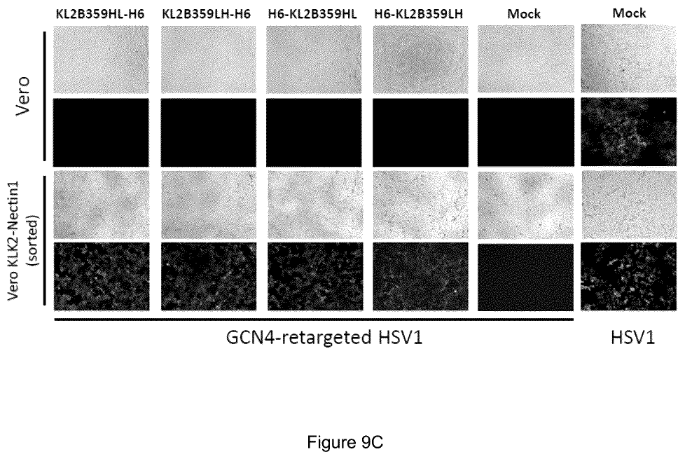

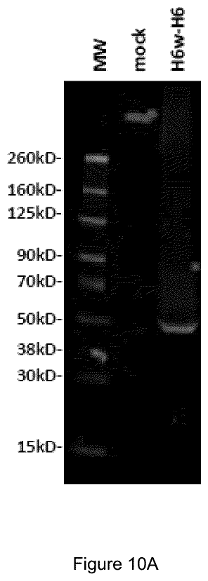

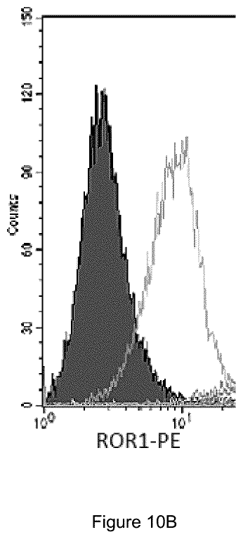

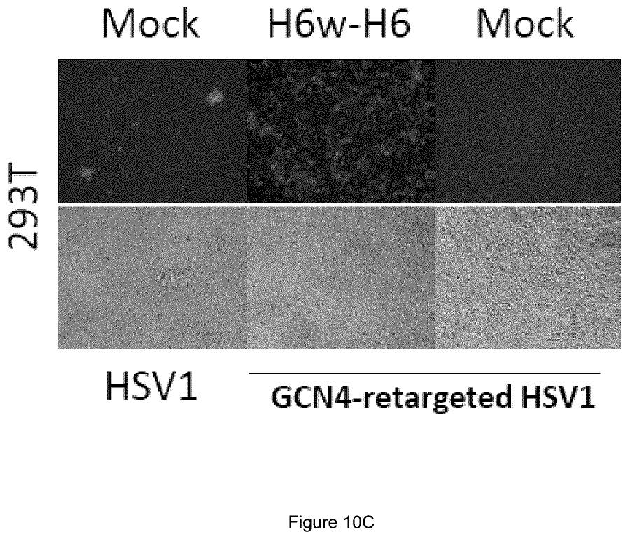

For retargeting to tumor markers, bispecific adaptor proteins were designed by fusing the anti-GCN4 H6 scFv to different single chain binders directed against the following targets: PSMA (FIG. 7), TMEFF2 (FIG. 8), KLK2 (FIG. 9) and ROR1 (FIG. 10). A list of all constructs is given in table 1. In the case of PSMA, it was demonstrated that supernatants of HEK293T cells transiently transfected by PSMA-H6 bispecific expression vectors (FIG. 7A) successfully retarget the infection of HEK293T expressing PSMA (FIGS. 7B and 7C) as well as of the PSMA positive prostate cancer cell line LNCaP, as monitored by GFP expression 24 hr post infection (FIG. 7C). Conversely, the bispecific adaptor proteins failed to retarget infection to the parental HEK293T cell line or to a PSMA negative prostate cancer cell line, DU145. For TMEFF2 (FIG. 8) similar results were observed. Supernatant of HEK293T cells transiently transfected by bispecific expression vectors (FIG. 8A) were able to redirect infection to Vero cell stably expressing TMEFF2 at their surface (FIGS. 8B and 8C) or to the TMEFF2 positive prostate cancer cell line 22Rv1 (FIG. 8C). The parental Vero cell line, lacking human TMEFF2 expression, was resistant to infection by the GCN4-retargeted virus. A Vero cell line expressing KLK2 tethered to the cell surface by the transmembrane and cytoplasmic domain of nectin1 (FIGS. 9B and 9C) was rendered sensitive to infection by a GCN4-retargeted HSV1 in presence of supernatants of HEK293T cell transfected with KLK2-H6 adaptor expression construct (FIG. 9A). In contrast, the parental Vero cell line was resistant. In another example, HEK293T cells, which express ROR1 at their surface (FIG. 10B), were susceptible to infection by the GCN4-retargeted HSV1 in presence of a supernatant of HEK293T cell transfected with an ROR1-H6 adaptor expression construct (FIG. 10C).

Altogether, these results demonstrate that retargeting HSV1 using the GCN4 peptide/H6 scFv pair is efficient and versatile. This could be easily adapted to different formats of binders (scFv, VHH, Darpin) with minimal engineering to a variety of tumor markers.

### HSV Retargeting by Leucine-Zipper RE/ER

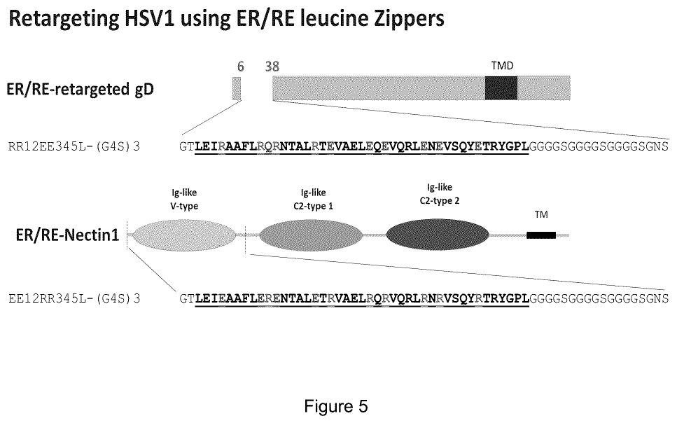

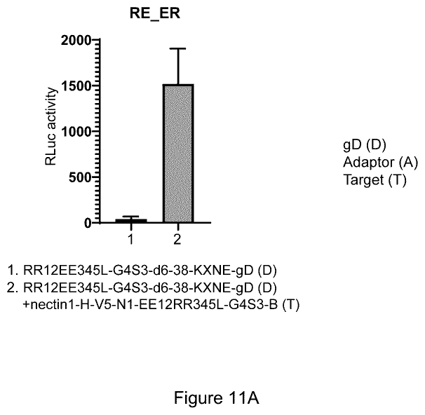

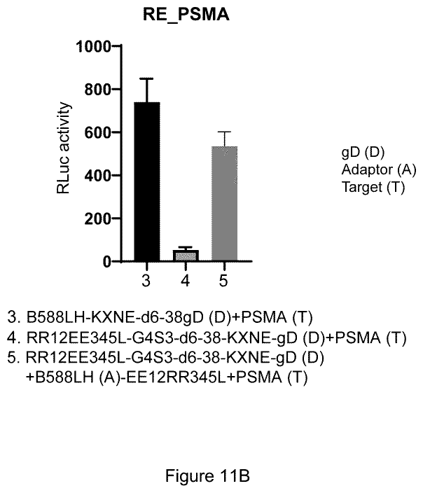

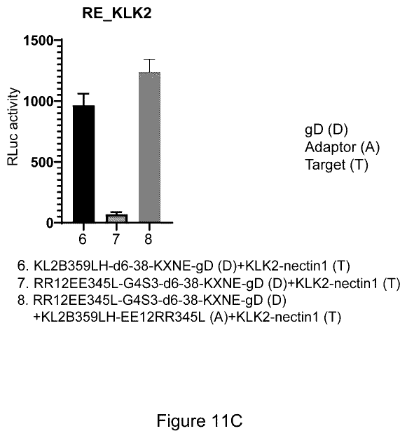

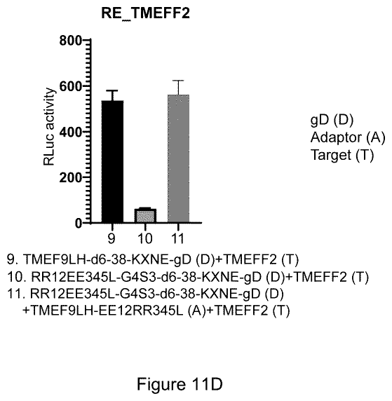

To demonstrate HSV1 retargeting using a leucine zipper pair (see FIG. 5), a direct in vitro fusion assay using a split-protein reporter system was developed. Briefly, a population of cells (effector cells) were transfected with i) a modified gD glycoprotein where the amino acids 6-36 were replaced with a leucine zipper of sequence (SEQ ID NO: 6) followed by a (G₄S)₃ linker (SEQ ID NO: 126) (referred to as RR12EE345L-(G₄S)₃-d6-38gD), with ii) the three other wild type glycoprotein components of the HSV1 membrane fusion machinery (gB, gH and gL) and with iii) one of the component of the split-protein reporter system pair (cDSP). Another population of cell (target cells) were transfected with a protein fusion where the EE12RR345L leucine zipper complementary to the RR12EE345L leucine zipper above (SEQ ID NO: 10) and a (G₄S)₃ linker (SEQ ID NO: 126) replace the AA 31-145 of human Nectin1 (referred to as EE12RR345L-(G₄S)₃-nectin1) and with the second component of the split-protein reporter system pair (nDSP). When the target and effector cells are put in contact, a robust luciferase activity can be measured indicating membrane fusion between effector and target cells and the subsequent reconstitution of the luciferase reporter (FIG. 11A). In comparison, when the EE12RR345L-(G₄S)₃-nectin1 receptor is omitted from the reaction, no fusion is detected indicating that fusion requires the presence of EE12RR345L-(G₄S)₃-nectin1. As HEK293T cells naturally express human Nectin1, the control reaction also showed that RR12EE345L-(G₄S)₃-d6-38gD has lost its tropism for its natural receptor nectin1.

In order to demonstrate HSV1 retargeting to specific tumor markers using bispecific adaptors, the in vitro fusion assay was then repeated in an experiment where transfection of EE12RR345L-(G₄S)₃-nectin1 in the target cells was replaced by transfection of the specific tumor marker of interest (PSMA, KLK2-nectin1 fusion, and TMEFF2) and a secreted bispecific adaptor composed of the corresponding binding protein (B588LH, KL2B359LH, and TMEF9LH respectively) fused to the EE12RR345L leucine zipper by a GGGGS linker (SEQ ID NO: 124) (See Table 1). As a negative control, the bispecific adaptor was omitted from the target cell reactions. As a positive control effector cells were transfected with a modified gD glycoprotein where the amino acids 6-36 were replaced by the corresponding tumor marker binding protein (B588LH-d6-38gD, KL2B359LH-d6-38gD, and TMEF9LH-d6-38gD respectively) instead of RR12EE345L-(G₄S)₃-d6-38gD and the bispecific adaptor was omitted from the target cell transfection. As shown in FIGS. 11B to 11D, the presence of the bispecific adaptor efficiently induces membrane fusion between the target and effector cells as measured by luciferase activity (right column) in a comparable fashion as their respective control (left column). On the contrary, in the absence of bispecific adaptor no fusion is detected (middle column). This confirms that the fusion is specific and mediated by the bispecific adaptor.

All together the data in FIGS. 11A-11D demonstrate that membrane fusion through HSV1 glycoprotein fusion machinery can be retargeted in the same way as with the GCN4 peptide/H6 scFV pair using a pair of complementary leucine zippers instead, broadening the scope of the HSV1 retargeting strategy using bispecific adaptors.

HSV Retargeting by La Epitope/5B9HL scFv

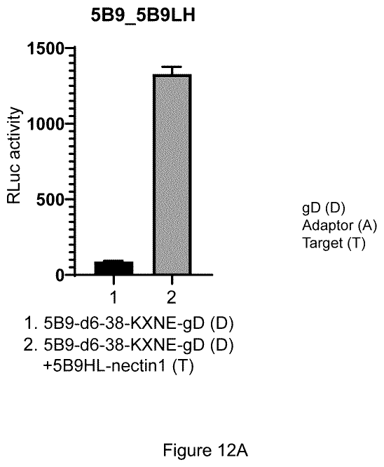

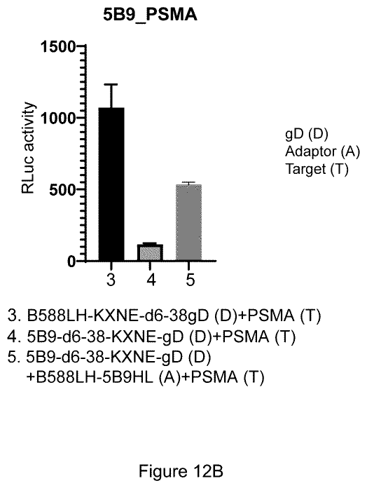

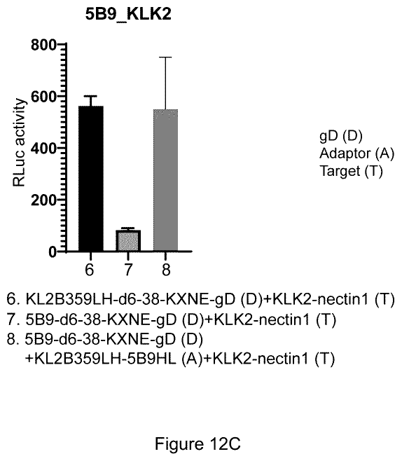

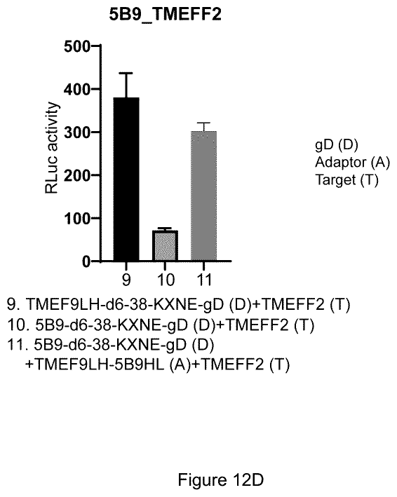

To demonstrate HSV1 retargeting using a different peptide/scFv pair, a direct in vitro fusion assay using a split-protein reporter system was developed. Briefly, a population of cells (effector cells) were transfected with i) a modified gD glycoprotein where the amino acids 6-36 were replaced with an La epitope (SEQ ID NO: 12)) flanked by two linkers (final sequence: GTGSKPLPEVTDEYGGGGSGNS (SEQ ID NO: 13)) and referred to as La-d6-38gD, with ii) the three other wild type glycoprotein components of the HSV1 membrane fusion machinery (gB, gH and gL) and with iii) one of the component of the split-protein reporter system pair (cDSP). Another population of cell (target cells) were transfected with a protein fusion where the 5B9HL scFv (SEQ: QVQLVQSGAEVKKPGASVKVSCKASGYTFTHYYIYWVRQAPGQGLEWMGGVNP SNGGTHFNEKFKSRVTMTRDTSISTAYMELSRLRSDDTAVYYCARSEYDYGLGFA YWGQGTLVTVSSGGSEGKSSGSGSESKSTGGSDIVMTQSPDSLAVSLGERATINCK SSQSLLNSRTPKNYLAWYQQKPGQPPKLLIYWASTRKSGVPDRFSGSGSGTDFTLT ISSLQAEDVAVYYCKQSYNLLTFGGGTKVEIK (SEQ ID NO: 34)) followed by a G₄S linker (SEQ ID NO: 124) replace the AA 31-145 of human Nectin1 (referred to as 5B9HL-nectin1) and with the second component of the split-protein reporter system pair (nDSP). When the target and effector cells are put in contact, a robust luciferase activity can be measured indicating membrane fusion between effector and target cells and the subsequent reconstitution of the luciferase reporter (FIG. 12A). In comparison, when the 5B9HL-nectin 1 receptor is omitted from the target cell transfection, no fusion is detected indicating that fusion requires the presence of 5B9HL-nectin1. As HEK293T cells naturally express human Nectin1, the control reaction also shows that La-d6-38gD has lost its tropism for its natural receptor nectin1.

In order to demonstrate HSV1 retargeting to specific tumor markers using bispecific adaptors, the in vitro fusion assay was then repeated in an experiment where transfection of 5B9HL-nectin 1 in the target cells was replaced by transfection of the specific tumor marker of interest (PSMA, KLK2-nectin1 fusion, and TMEFF2) and a secreted bispecific adaptor comprised of the corresponding binding protein (B588LH, KL2B359LH, and TMEF9LH respectively) fused to the 5B9HL scFv by a GGGGS linker (SEQ ID NO: 124) (see Table 1). As a negative control, the bispecific adaptor was omitted from the target cell reactions. As a positive control effector cells were transfected with a modified gD glycoprotein where the amino acids 6-36 were replaced by the corresponding tumor marker binding protein (B588LH-d6-38gD, KL2B359LH-d6-38gD, and TMEF9LH-d6-38gD respectively) instead of La-d6-38gD and the bispecific adaptor was omitted from the target cell transfection. As shown in FIGS. 12B-12D, the presence of the bispecific adaptor efficiently induces membrane fusion between the target and effector cells as measured by luciferase activity (right column) in a comparable fashion as their respective control (left column). On the contrary, in the absence of bispecific adaptor no fusion is detected (middle column). This confirms that the fusion is specific and mediated by the bispecific adaptor.

All together the data in FIGS. 12A-12D demonstrate that membrane fusion through HSV1 glycoprotein fusion machinery can be retargeted in the same way as with the GCN4 peptide/H6 scFV pair using a different peptide/scFv pair, here La/5B9HL scFv, broadening the scope of the HSV1 retargeting strategy using bispecific adaptors.

TABLE 1
Bispecific tested herein

| | | Nterm | | Cterm | |
|---|---|---|---|---|---|
| Name | Target | binder | Linker | binder | SEQ ID NOS |
| B116-H6 | PSMA | VHH | NSGGGGS | H6 scFv | METDTLLLWVLLLWVPGSTGDQLQLVESGGGLVHAGGSLRLS |
| | | | (SEQ ID NO: | | CAASGSTFSINAIGWYRQAPGKQRELVAALSSGGSKNYADSV |
| | | | 123 | | KGRFTISRDNAKNTVYLQMNRLKPEDTAVYYCNAEIYYSDGVD |
| | | | | | DGYRGMDYWGKGTQVTVSSNSGGGGSDAVVTQESALTTSP |
| | | | | | GETVTLTCRSSTGAVTTSNYASWVQEKPDHLFTGLIGGTNNR |
| | | | | | APGVPARFSGSLIGDKAALTITGAQTEDEAIYFCALWYSNHWV |
| | | | | | FGGGTKLTVLGGGGGSGGGGSGGGGSGGGGSDVQLQQSG |
| | | | | | PGLVAPSQSLSITCTVSGFSLTDYGVNWVRQSPGKGLEWLGV |
| | | | | | IWGDGITDYNSALKSRLSVTKDNSKSQVFLKMNSLQSGDSAR |
| | | | | | YYCVTGLFDYWGQGTTLTVSSGGGGSLESRGPFEQKLISEED |
| | | | | | LNMHTGHHHHHH (SEQ ID NO: 87) |
| | | | | | ATGGAGACCGACACACTGCTGCTGTGGGTGCTGCTGCTGT |
| | | | | | GGGTGCCCGGCTCTACAGGCGATCAGCTGCAGCTGGTGG |
| | | | | | AGAGCGGAGGAGGCCTGGTGCACGCAGGAGGCAGCCTGA |
| | | | | | GGCTGTCCTGCGCAGCATCTGGCAGCACCTTCAGCATCAA |
| | | | | | CGCAATCGGATGGTACAGGCAGGCACCTGGCAAGCAGAG |
| | | | | | GGAGCTGGTGGCCGCCCTGAGCTCCGGCGGCAGCAAGAA |
| | | | | | TTACGCCGACTCCGTGAAGGGCCGGTTTACAATCAGCAGA |
| | | | | | GATAACGCCAAGAATACCGTGTATCTGCAGATGAACAGGCT |
| | | | | | GAAGCCAGAGGACACCGCCGTGTACTATTGCAATGCCGAG |
| | | | | | ATCTACTATTCCGACGGAGTGGACGATGGCTACCGCGGAA |
| | | | | | TGGATTATTGGGGCAAGGGCACACAGGTGACCGTGTCTTC |
| | | | | | GAATTCTGGAGGAGGAGGCTCTGACGCAGTGGTGACACAG |
| | | | | | GAGAGCGCCCTGACCACATCCCCTGGAGAGACCGTGACAC |
| | | | | | TGACCTGTCGCTCCTCTACCGGCGCCGTGACCACATCTAAT |
| | | | | | TATGCCAGCTGGGTGCAGGAGAAGCCAGATCACCTGTTCA |
| | | | | | CAGGCCTGATCGGAGGCACCAACAATAGGGCACCAGGCGT |
| | | | | | GCCTGCAAGATTTTCCGGCTCTCTGATCGGCGACAAGGCC |
| | | | | | GCCCTGACAATCACCGGAGCACAGACCGAGGATGAGGCCA |
| | | | | | TCTACTTCTGCGCCCTGTGGTATAGCAACCACTGGGTGTTT |
| | | | | | GGCGGCGGCACAAAGCTGACCGTGCTGGGAGGAGGAGGA |
| | | | | | GGCTCTGGAGGAGGAGGCAGCGGCGGCGGCGGCTCCGG |
| | | | | | CGGCGGCGGCTCTGACGTGCAGCTGCAGCAGTCCGGACC |
| | | | | | AGGCCTGGTGGCACCCAGCCAGTCCCTGTCTATCACATGT |
| | | | | | ACCGTGTCTGGCTTCAGCCTGACCGATTACGGAGTGAACT |
| | | | | | GGGTGCGGCAGTCCCCAGGCAAGGGACTGGAGTGGCTGG |
| | | | | | GCGTGATCTGGGGCGACGGCATCACAGATTATAATTCTGC |
| | | | | | CCTGAAGTCCCGGCTGTCTGTGACCAAGGATAACAGCAAG |
| | | | | | TCCCAGGTGTTCCTGAAGATGAATAGCCTGCAGTCCGGCG |
| | | | | | ACTCTGCCAGATACTATTGCGTGACAGGCCTGTTTGATTAC |
| | | | | | TGGGGCCAGGGCACCACACTGACCGTGAGCTCCGGAGGA |
| | | | | | GGAGGCTCCCTCGAGTCTAGAGGGCCCTTCGAACAAAAAC |
| | | | | | TCATCTCAGAAGAGGATCTGAATATGCATACCGGTCATCAT |
| | | | | | CACCATCACCATTGA (SEQ ID NO: 88) |
| H6-B110 | PSMA | H6 scFv | NSGGGGS | VHH | METDTLLLWVLLLWVPGSTGDTGDAVVTQESALTTSPGETVT |
| | | | (SEQ ID NO: | | LTCRSSTGAVTTSNYASWVQEKPDHLFTGLIGGTNNRAPGVP |
| | | | 123) | | ARFSGSLIGDKAALTITGAQTEDEAIYFCALWYSNHWVFGGGT |
| | | | | | KLTVLGGGGGSGGGGSGGGGSGGGGSDVQLQQSGPGLVA |
| | | | | | PSQSLSITCTVSGFSLTDYGVNWVRQSPGKGLEWLGVIWGD |
| | | | | | GITDYNSALKSRLSVTKDNSKSQVFLKMNSLQSGDSARYYCV |
| | | | | | TGLFDYWGQGTTLTVSSNSGGGGSEVQVVESGGGLVQTGG |
| | | | | | SLRLSCAASGPPLSSYAVAWFRQTPGKEREFVAAISWSGSNT |
| | | | | | YYADSVKGRFTISKDNAKNTVLVYLQMNSLKPEDTAVYYCAAD |
| | | | | | RRGGPLSDYEWEDEYADWGQGTQVTVSSGGGGSLESRGPF |
| | | | | | EQKLISEEDLNMHTGHHHHHH (SEQ ID NO: 89) |
| | | | | | ATGGAGACCGACACACTGCTGCTGTGGGTGCTGCTGCTGT |
| | | | | | GGGTGCCAGGCAGCACAGGCGACACCGGCGATGCAGTGG |
| | | | | | TGACACAGGAGAGCGCCCTGACCACATCCCCAGGAGAGAC |
| | | | | | CGTGACACTGACCTGCAGGAGCTCCACCGGAGCAGTGACC |
| | | | | | ACATCCAACTACGCCTCTTGGGTGCAGGAGAAGCCCGATC |
| | | | | | ACCTGTTCACAGGCCTGATCGGCGGCACCAACAATAGGGC |
| | | | | | ACCAGGCGTGCCCGCACGCTTTTCTGGCAGCCTGATCGGC |
| | | | | | GACAAGGCCGCCCTGACAATCACCGGAGCACAGACAGAGG |
| | | | | | ATGAGGCCATCTACTTCTGCGCCCTGTGGTATAGCAATCAC |
| | | | | | TGGGTGTTTGGCGGCGGCACAAAGCTGACCGTGCTGGGA |
| | | | | | GGAGGAGGAGGCTCTGGAGGAGGAGGCAGCGGCGGCGG |
| | | | | | CGGCTCCGGCGGCGGCGGCTCTGACGTGCAGCTGCAGCA |
| | | | | | GTCCGGACCTGGCCTGGTGGCACCATCCCAGTCTCTGAGC |
| | | | | | ATCACATGTACCGTGAGCGGCTTCTCCCTGACCGATTACGG |
| | | | | | AGTGAACTGGGTGCGGCAGTCCCCTGGCAAGGGACTGGA |
| | | | | | GTGGCTGGGCGTGATCTGGGGCGACGGCATCACAGATTAT |
| | | | | | AATTCTGCCCTGAAGTCTAGGCTGAGCGTGACCAAGGACA |
| | | | | | ACTCCAAGTCTCAGGTGTTCCTGAAGATGAACAGCCTGCAG |
| | | | | | TCTGGCGACAGCGCCCGCTACTATTGCGTGACAGGCCTGT |
| | | | | | TTGATTACTGGGGCCAGGGCACCACACTGACCGTGTCTTC |
| | | | | | GAATTCTGGAGGAGGAGGCTCCGAGGTGCAGGTGGTGGA |
| | | | | | GAGCGGAGGAGGCCTGGTGCAGACCGGAGGCAGCCTGCG |
| | | | | | GCTGTCCTGTGCAGCATCTGGACCACCTCTGTCCTCTTATG |
| | | | | | CAGTGGCATGGTTCAGGCAGACACCAGGCAAGGAGAGAGA |
| | | | | | GTTTGTGGCCGCCATCAGCTGGTCCGGCTCTAACACCTACT |
| | | | | | ATGCCGACTCTGTGAAGGGCCGGTTCACCATCAGCAAGGA |
| | | | | | TAACGCCAAGAATACCGTGCTGGTGTACCTGCAGATGAATA |
| | | | | | GCCTGAAGCCCGAGGATACCGCCGTGTACTATTGTGCAGC |
| | | | | | AGACAGGAGAGGAGGACCTCTGTCCGATTACGAGTGGGAG |
| | | | | | GACGAGTATGCCGATTGGGGCCAGGGCACACAGGTGACC |
| | | | | | GTGAGCTCCGGAGGAGGAGGCTCCCTCGAGTCTAGAGGG |
| | | | | | CCCTTCGAACAAAAACTCATCTCAGAAGAGGATCTGAATAT |
| | | | | | GCATACCGGTCATCATCACCATCACCATTGA (SEQ ID NO: |
| | | | | | 90) |
| B588LH-H6 | PSMA | VL-VH | GGGGS | H6 scFv | METDTLLLWVLLLWVPGSTGDTGQSVLTQPPSVSGAPGQRV |
| | | scFv | (SEQ ID NO | | TISCTGSSSNIGAGYDVHWYQQLPGTAPKLLIYGNTNRPSGVP |
| | | | 124) | | DRFSGSKSGTSASLAITGLQAEDEADYYCQSYDSSLSGTPYV |
| | | | | | VFGGGTKLTVLGGSEGKSSGSGSESKSTGGSEVQLVESGGG |
| | | | | | LVQPGGSLRLSCAASGFTFSFYNMNWVRQAPGKGLEWISYIS |
| | | | | | TSSSTIYYADSVKGRFTISRDNAKNSLYLQMNSLRDEDTAVYY |
| | | | | | CAREGSYYDSSGYPYYYYDMDVWGQGTTVTVSSGGGGSDA |
| | | | | | WTQESALTTSPGETVTLTCRSSTGAVTTSNYASWVQEKPDH |
| | | | | | LFTGLIGGTNNRAPGVPARFSGSLIGDKAALTITGAQTEDEAIY |
| | | | | | FCALWYSNHWVFGGGTKLTVLGGGGGSGGGGSGGGGSGG |
| | | | | | GGSDVQLQQSGPGLVAPSQSLSITCTVSGFSLTDYGVNWVR |
| | | | | | QSPGKGLEWLGVIWGDGITDYNSALKSRLSVTKDNSKSQVFL |
| | | | | | KMNSLQSGDSARYYCVTGLFDYWGQGTTLTVSSLESRGPFE |
| | | | | | QKLISEEDLNMHTGHHHHHH (SEQ ID NO: 91) |
| | | | | | ATGGAGACCGACACACTGCTGCTGTGGGTGCTGCTGCTGT |
| | | | | | GGGTGCCTGGCTCCACAGGCGATACCGGACAGTCTGTGCT |
| | | | | | GACCCAGCCACCTAGCGTGTCCGGAGCACCAGGCCAGCG |
| | | | | | GGTGACAATCTCCTGCACCGGCAGCTCCTCTAACATCGGC |
| | | | | | GCCGGCTACGACGTGCACTGGTATCAGCAGCTGCCTGGCA |
| | | | | | CAGCCCCAAAGCTGCTGATCTACGGCAACACCAATAGGCC |
| | | | | | CAGCGGCGTGCCTGATCGCTTTTCTGGCAGCAAGTCCGGC |
| | | | | | ACATCTGCCAGCCTGGCAATCACCGGACTGCAGGCAGAGG |
| | | | | | ACGAGGCCGATTACTATTGCCAGTCTTACGACAGCTCCCTG |
| | | | | | AGCGGCACACCTTATGTGGTGTTCGGAGGAGGCACAAAGC |
| | | | | | TGACCGTGCTGGGAGGCAGCGAGGGCAAGTCTAGCGGCT |
| | | | | | CCGGCTCTGAGAGCAAGTCCACCGGAGGCAGCGAGGTGC |
| | | | | | AGCTGGTGGAGTCCGGAGGAGGCCTGGTGCAGCCAGGAG |
| | | | | | GCAGCCTGCGGCTGTCCTGTGCCGCCTCTGGCTTCACCTT |
| | | | | | TTCCTTCTACAACATGAATTGGGTGAGACAGGCACCTGGCA |
| | | | | | AGGGCCTGGAGTGGATCAGCTATATCTCCACATCCTCTAGC |
| | | | | | ACCATCTACTATGCCGACAGCGTGAAGGGCCGGTTTACAAT |
| | | | | | CAGCCGGGACAACGCCAAGAATAGCCTGTACCTGCAGATG |
| | | | | | AACAGCCTGAGGGACGAGGATACCGCCGTGTACTATTGCG |
| | | | | | CCCGCGAGGGCTCCTACTATGACTCCTCTGGCTATCCATAC |
| | | | | | TATTACTATGACATGGACGTGTGGGGCCAGGGCACCACAG |
| | | | | | TGACAGTGAGCTCCGGCGGAGGAGGCAGCGATGCAGTGG |
| | | | | | TGACCCAGGAGTCTGCCCTGACCACAAGCCCAGGCGAGAC |
| | | | | | CGTGACACTGACCTGTCGGTCTAGCACCGGCGCCGTGACC |
| | | | | | ACAAGCAACTACGCCTCCTGGGTGCAGGAGAAGCCCGACC |
| | | | | | ACCTGTTTACAGGCCTGATCGGAGGCACCAACAATAGGGC |
| | | | | | ACCAGGCGTGCCCGCAAGATTCTCTGGCAGCCTGATCGGC |
| | | | | | GACAAGGCCGCCCTGACAATCACCGGAGCACAGACCGAG |
| | | | | | GATGAGGCCATCTACTTTTGCGCCCTGTGGTATTCCAATCA |
| | | | | | CTGGGTGTTCGGCGGCGGCACAAAGCTGACCGTGCTGGG |
| | | | | | TGGAGGAGGAGGCTCCGGAGGAGGAGGCTCTGGCGGCGG |
| | | | | | CGGCAGCGGAGGCGGCGGCTCCGACGTGCAGCTGCAGCA |
| | | | | | GAGCGGACCAGGCCTGGTGGCACCATCCCAGTCTCTGAGC |
| | | | | | ATCACATGTACCGTGTCTGGCTTCAGCCTGACCGATTACGG |
| | | | | | CGTGAACTGGGTGAGACAGTCTCCAGGCAAGGGCCTGGAG |
| | | | | | TGGCTGGGCGTGATCTGGGGCGACGGCATCACAGATTATA |
| | | | | | ATAGCGCCCTGAAGTCCAGGCTGTCTGTGACCAAGGATAA |
| | | | | | CTCCAAGTCTCAGGTGTTTCTGAAGATGAATAGCCTGCAGT |
| | | | | | CCGGCGACTCTGCCCGCTACTATTGCGTGACAGGCCTGTT |
| | | | | | CGATTACTGGGGACAGGGCACCACACTGACCGTGTCCTCT |
| | | | | | CTCGAGTCTAGAGGGCCCTTCGAACAAAAACTCATCTCAGA |
| | | | | | AGAGGATCTGAATATGCATACCGGTCATCATCACCATCACC |
| | | | | | ATTGA (SEQ ID NO: 92) |
| B588HL-H6 | PSMA | VH-VL | GGGGS | H6 scFv | METDTLLLWVLLLWVPGSTGDTGEVQLVESGGGLVQPGGSL |
| | | scFv | (SEQ ID | | RLSCAASGFTFSFYNMNWVRQAPGKGLEWISYISTSSSTIYYA |
| | | | NO: 124) | | DSVKGRFTISRDNAKNSLYLQMNSLRDEDTAVYYCAREGSYY |
| | | | | | DSSGYPYYYYDMDVWGQGTTVTVSSGGSEGKSSGSGSESK |
| | | | | | STGGSQSVLTQPPSVSGAPGQRVTISCTGSSSNIGAGYDVHW |
| | | | | | YQQLPGTAPKLLIYGNTNRPSGVPDRFSGSKSGTSASLAITGL |
| | | | | | QAEDEADYYCQSYDSSLSGTPYVVFGGGTKLTVLGGGGSDA |
| | | | | | VVTQESALTTSPGETVTLTCRSSTGAVTTSNYASWVQEKPDH |
| | | | | | LFTGLIGGTNNRAPGVPARFSGSLIGDKAALTITGAQTEDEAIY |
| | | | | | FCALWYSNHWVFGGGTKLTVLGGGGGSGGGGGGGGSGG |
| | | | | | GGSDVQLQQSGPGLVAPSQSLSITCTVSGFSLTDYGVNWVR |
| | | | | | QSPGKGLEWLGVIWGDGITDYNSALKSRLSVTKDNSKSQVFL |
| | | | | | KMNSLQSGDSARYYCVTGLFDYWGQGTTLTVSSLESRGPFE |
| | | | | | QKLISEEDLNMHTGHHHHHH (SEQ ID NO: 93) |
| | | | | | ATGGAGACCGACACACTGCTGCTGTGGGTGCTGCTGCTGT |
| | | | | | GGGTGCCTGGCAGCACAGGCGATACCGGAGAGGTGCAGC |
| | | | | | TGGTGGAGTCCGGAGGAGGCCTGGTGCAGCCAGGAGGCT |
| | | | | | CTCTGAGGCTGAGCTGCGCAGCATCCGGCTTCACCTTTTC |
| | | | | | CTTCTACAACATGAATTGGGTGAGACAGGCACCAGGCAAG |
| | | | | | GGCCTGGAGTGGATCTCTTATATCAGCACAAGCTCCTCTAC |
| | | | | | CATCTACTATGCCGACAGCGTGAAGGGCCGGTTTACAATCA |
| | | | | | GCAGAGATAACGCCAAGAACAGCCTGTACCTGCAGATGAA |
| | | | | | CTCTCTGAGGGACGAGGATACCGCCGTGTACTATTGTGCC |
| | | | | | CGCGAGGGCTCCTACTATGACAGCTCCGGCTATCCATACTA |
| | | | | | TTACTATGACATGGACGTGTGGGGCCAGGGCACCACAGTG |
| | | | | | ACCGTGTCTAGCGGAGGCAGCGAGGGCAAGTCCTCTGGCA |
| | | | | | GCGGCTCCGAGTCTAAGAGCACAGGAGGCTCCCAGTCTGT |
| | | | | | GCTGACCCAGCCACCTAGCGTGTCCGGAGCACCAGGCCA |
| | | | | | GCGGGTGACAATCTCCTGCACCGGCAGCTCCTCTAATATC |
| | | | | | GGCGCCGGCTACGACGTGCACTGGTATCAGCAGCTGCCTG |
| | | | | | GCACAGCCCCAAAGCTGCTGATCTACGGCAACACCAATAG |
| | | | | | GCCCAGCGGCGTGCCTGATCGCTTTTCTGGCAGCAAGTCC |
| | | | | | GGCACATCTGCCAGCCTGGCAATCACCGGACTGCAGGCAG |
| | | | | | AGGACGAGGCCGATTACTATTGCCAGTCCTACGACAGCTC |
| | | | | | CCTGTCTGGCACCCCTTATGTGGTGTTCGGCGGCGGCACA |
| | | | | | AAGCTGACCGTGCTGGGAGGAGGAGGCAGCGATGCAGTG |
| | | | | | GTGACACAGGAGTCCGCCCTGACCACATCTCCAGGAGAGA |
| | | | | | CCGTGACACTGACCTGTAGATCTAGCACCGGCGCCGTGAC |
| | | | | | CACATCTAACTACGCCAGCTGGGTGCAGGAGAAGCCTGAC |
| | | | | | CACCTGTTTACAGGCCTGATCGGAGGCACCAACAATAGGG |
| | | | | | CACCAGGCGTGCCCGCAAGATTCTCCGGCTCTCTGATCGG |
| | | | | | CGACAAGGCCGCCCTGACAATCACCGGAGCACAGACCGAG |
| | | | | | GATGAGGCCATCTACTTTTGCGCCCTGTGGTATTCCAATCA |
| | | | | | CTGGGTCTTTGGAGGAGGCACAAAGCTGACCGTGCTGGGT |
| | | | | | GGAGGAGGAGGCAGCGGCGGAGGAGGCTCCGGAGGCGG |
| | | | | | CGGCTCTGGCGGCGGCGGCAGCGACGTGCAGCTGCAGCA |
| | | | | | GAGCGGACCAGGCCTGGTGGCACCCAGCCAGTCCCTGTCT |
| | | | | | ATCACATGTACCGTGTCCGGCTTCTCTCTGACCGATTACGG |
| | | | | | CGTGAACTGGGTGCGGCAGTCTCCTGGCAAGGGCCTGGA |
| | | | | | GTGGCTGGGCGTGATCTGGGGCGACGGCATCACAGATTAT |
| | | | | | AATAGCGCCCTGAAGAGCAGGCTGTCCGTGACCAAGGATA |
| | | | | | ACAGCAAGTCCCAGGTGTTTCTGAAGATGAACAGCCTGCA |
| | | | | | GAGCGGCGACTCCGCCCGCTACTATTGCGTGACAGGCCTG |
| | | | | | TTCGATTACTGGGGACAGGGCACCACACTGACCGTGTCCT |
| | | | | | CTCTCGAGTCTAGAGGGCCCTTCGAACAAAAACTCATCTCA |
| | | | | | GAAGAGGATCTGAATATGCATACCGGTCATCATCACCATCA |
| | | | | | CCATTGA (SEQ ID NO: 94) |
| TMEF847LH- | TMEFF2 | VL-VH | NSGGGGS | H6 scFv | MAWVWTLLFLMAAAQSIQADIQMTQSPSSLSASVGDRVTITC |
| H6 | | scFv | (SEQ ID NO: | | RASQGIRNDLGWYQQKPGKAPKLLIYAASSLQSGVPSRFSGS |
| | | | 123) | | GSGTDFTLTISSLQPEDFATYYCLQDYNYPLTFGGGTKVEIKG |
| | | | | | GSEGKSSGSGSESKSTGGSEVQLLESGGGLVQPGGSLRLSC |
| | | | | | AASGFTFSSYSMSWVRQAPGKGLEWVSVISGSGGFTDYADS |
| | | | | | VKGRFTISRDNSKNTLYLQMNSLRAEDTAVYYCARMPLNSPH |
| | | | | | DYWGQGTLVTVSSNSGGGGSDAVVTQESALTTSPGETVTLT |
| | | | | | CRSSTGAVTTSNYASWVQEKPDHLFTGLIGGTNNRAPGVPAR |
| | | | | | FSGSLIGDKAALTITGAQTEDEAIYFCALWYSNHWVFGGGTKL |
| | | | | | TVLGGGGGSGGGGSGGGGSGGGGSDVQLQQSGPGLVAPS |
| | | | | | QSLSITCTVSGFSLTDYGVNWVRQSPGKGLEWLGVIWGDGIT |
| | | | | | DYNSALKSRLSVTKDNSKSQVFLKMNSLQSGDSARYYCVTGL |
| | | | | | FDYWGQGTTLTVSSLESRGPFEQKLISEEDLNMHTGHHHHHH |
| | | | | | (SEQ ID NO: 95) |
| | | | | | ATGGCCTGGGTGTGGACCCTGCTGTTCCTGATGGCAGCAG |
| | | | | | CACAGTCCATCCAGGCCGACATCCAGATGACACAGTCTCC |
| | | | | | AAGCTCCCTGAGCGCCTCCGTGGGCGACAGGGTGACCATC |
| | | | | | ACATGCAGGGCAAGCCAGGGCATCCGGAACGATCTGGGCT |
| | | | | | GGTACCAGCAGAAGCCCGGCAAGGCCCCTAAGCTGCTGAT |
| | | | | | CTATGCAGCATCTAGCCTGCAGTCCGGAGTGCCATCTCGG |
| | | | | | TTCTCTGGCAGCGGCTCCGGAACCGACTTCACCCTGACAA |
| | | | | | TCTCCTCTCTGCAGCCTGAGGACTTCGCCACATACTATTGC |
| | | | | | CTGCAGGATTACAATTATCCACTGACCTTTGGCGGCGGCAC |
| | | | | | AAAGGTGGAGATCAAGGGAGGCTCCGAGGGCAAGAGCTC |
| | | | | | CGGCTCTGGCAGCGAGTCCAAGTCTACCGGCGGCTCTGAG |
| | | | | | GTGCAGCTGCTGGAGAGCGGAGGAGGACTGGTGCAGCCA |
| | | | | | GGAGGCAGCCTGCGCCTGTCCTGTGCCGCCTCTGGCTTCA |
| | | | | | CCTTTTCTAGCTACAGCATGTCCTGGGTGCGGCAGGCACC |
| | | | | | TGGCAAGGGACTGGAGTGGGTGAGCGTGATCTCTGGCAGC |
| | | | | | GGCGGCTTCACAGACTACGCCGATTCCGTGAAGGGCCGGT |
| | | | | | TTACCATCAGCAGAGACAACTCCAAGAATACACTGTATCTG |
| | | | | | CAGATGAACAGCCTGAGAGCCGAGGACACCGCCGTGTACT |
| | | | | | ATTGTGCCAGGATGCCACTGAACTCTCCCCACGATTATTGG |
| | | | | | GGCCAGGGCACCCTGGTGACAGTGTCCTCTAATTCCGGCG |
| | | | | | GCGGCGGATCCGATGCAGTGGTGACACAGGAGTCCGCCC |
| | | | | | TGACCACATCTCCAGGAGAGACCGTGACACTGACCTGTAG |
| | | | | | ATCTAGCACCGGCGCCGTGACCACATCTAACTACGCCAGC |
| | | | | | TGGGTGCAGGAGAAGCCTGACCACCTGTTTACAGGCCTGA |
| | | | | | TCGGAGGCACCAACAATAGGGCACCAGGCGTGCCCGCAA |
| | | | | | GATTCTCCGGCTCTCTGATCGGCGACAAGGCCGCCCTGAC |
| | | | | | AATCACCGGAGCACAGACCGAGGATGAGGCCATCTACTTTT |
| | | | | | GCGCCCTGTGGTATTCCAATCACTGGGTCTTTGGAGGAGG |
| | | | | | CACAAAGCTGACCGTGCTGGGTGGAGGAGGAGGCAGCGG |
| | | | | | CGGAGGAGGCTCCGGAGGCGGCGGCTCTGGCGGCGGCG |
| | | | | | GCAGCGACGTGCAGCTGCAGCAGAGCGGACCAGGCCTGG |
| | | | | | TGGCACCCAGCCAGTCCCTGTCTATCACATGTACCGTGTCC |
| | | | | | GGCTTCTCTCTGACCGATTACGGCGTGAACTGGGTGCGGC |
| | | | | | AGTCTCCTGGCAAGGGCCTGGAGTGGCTGGGCGTGATCTG |
| | | | | | GGGCGACGGCATCACAGATTATAATAGCGCCCTGAAGAGC |
| | | | | | AGGCTGTCCGTGACCAAGGATAACAGCAAGTCCCAGGTGT |
| | | | | | TTCTGAAGATGAACAGCCTGCAGAGCGGCGACTCCGCCCG |
| | | | | | CTACTATTGCGTGACAGGCCTGTTCGATTACTGGGGACAG |
| | | | | | GGCACCACACTGACCGTGTCCTCTCTCGAGTCTAGAGGGC |
| | | | | | CCTTCGAACAAAAACTCATCTCAGAAGAGGATCTGAATATG |
| | | | | | CATACCGGTCATCATCACCATCACCATTGA (SEQ ID |
| | | | | | NO: 96) |
| TMEF9HL- | TMEFF2 | VH-VL | NSGGGGS | H6 scFv | MAWVWTLLFLMAAAQSIQAQVQLQESGPGLVKPSETLSLTCT |
| H6 | | scFv | (SEQ ID NO: | | VSGVSISSYFWSWLRQPAGKGLQWIGRISTSGSTNHNPSLKS |
| | | | 123) | | RVIMSVDTSKNQFSLKLSSVTAADTAVYYCVRDWTGFDYWG |
| | | | | | QGTLVTVSSGGSEGKSSGSGSESKSTGGSSYELTQPASVSG |
| | | | | | SPGQSITISCIGTSSDVGSYNLVSWYQQHPGKVPKLMIYEGSK |
| | | | | | RPSGVSNRFSGSKSGNTASLTISGLQAEDEADYYCSSYAGSS |
| | | | | | TYVFGTGTKVTVLNSGGGGSDAVVTQESALTTSPGETVTLTC |
| | | | | | RSSTGAVTTSNYASWVQEKPDHLFTGLIGGTNNRAPGVPARF |
| | | | | | SGSLIGDKAALTITGAQTEDEAIYFCALWYSNHWVFGGGTKLT |
| | | | | | VLGGGGGSGGGGSGGGGSGGGGSDVQLQQSGPGLVAPSQ |
| | | | | | SLSITCTVSGFSLTDYGVNWVRQSPGKGLEWLGVIWGDGITD |
| | | | | | YNSALKSRLSVTKDNSKSQVFLKMNSLQSGDSARYYCVTGLF |
| | | | | | DYWGQGTTLTVSSLESRGPFEQKLISEEDLNMHTGHHHHHH |
| | | | | | (SEQ ID NO: 97) |
| | | | | | ATGGCCTGGGTGTGGACCCTGCTGTTCCTGATGGCAGCAG |
| | | | | | CACAGTCCATCCAGGCACAGGTGCAGCTGCAGGAGAGCG |
| | | | | | GACCAGGACTGGTGAAGCCATCTGAGACCCTGAGCCTGAC |
| | | | | | CTGCACAGTGTCTGGCGTGAGCATCAGCTCCTACTTTTGGA |
| | | | | | GCTGGCTGAGGCAGCCAGCAGGCAAGGGACTGCAGTGGA |
| | | | | | TCGGCCGCATCTCCACCTCTGGCAGCACAAACCACAATCCT |
| | | | | | TCCCTGAAGTCTAGAGTGATCATGTCCGTGGACACCTCTAA |
| | | | | | GAACCAGTTCTCCCTGAAGCTGTCTAGCGTGACCGCCGCC |
| | | | | | GATACAGCCGTGTACTATTGCGTGAGGGACTGGACAGGCT |
| | | | | | TTGATTACTGGGGCCAGGGCACCCTGGTGACAGTGTCCTC |
| | | | | | TGGAGGCAGCGAGGGCAAGAGCTCCGGCTCCGGCTCTGA |
| | | | | | GAGCAAGTCCACCGGCGGCTCTAGCTATGAGCTGACACAG |
| | | | | | CCTGCATCTGTGAGCGGCTCCCCAGGACAGAGCATCACCA |
| | | | | | TCTCCTGTATCGGCACATCCTCTGACGTGGGCTCCTACAAC |
| | | | | | CTGGTGTCTTGGTATCAGCAGCACCCCGGCAAGGTGCCTA |
| | | | | | AGCTGATGATCTATGAGGGCTCCAAGAGGCCAAGCGGCGT |
| | | | | | GTCCAACAGATTCTCTGGCAGCAAGTCCGGCAATACCGCC |
| | | | | | TCTCTGACAATCAGCGGACTGCAGGCAGAGGACGAGGCAG |
| | | | | | ATTACTATTGTAGCTCCTACGCCGGCTCTAGCACCTACGTG |
| | | | | | TTCGGCACCGGCACAAAGGTGACAGTGCTGAATAGCGGCG |
| | | | | | GCGGCGGATCCGATGCAGTGGTGACACAGGAGTCCGCCC |
| | | | | | TGACCACATCTCCAGGAGAGACCGTGACACTGACCTGTAG |
| | | | | | ATCTAGCACCGGCGCCGTGACCACATCTAACTACGCCAGC |
| | | | | | TGGGTGCAGGAGAAGCCTGACCACCTGTTTACAGGCCTGA |
| | | | | | TCGGAGGCACCAACAATAGGGCACCAGGCGTGCCCGCAA |
| | | | | | GATTCTCCGGCTCTCTGATCGGCGACAAGGCCGCCCTGAC |
| | | | | | AATCACCGGAGCACAGACCGAGGATGAGGCCATCTACTTTT |
| | | | | | GCGCCCTGTGGTATTCCAATCACTGGGTCTTTGGAGGAGG |
| | | | | | CACAAAGCTGACCGTGCTGGGTGGAGGAGGAGGCAGCGG |
| | | | | | CGGAGGAGGCTCCGGAGGCGGCGGCTCTGGCGGCGGCG |
| | | | | | GCAGCGACGTGCAGCTGCAGCAGAGCGGACCAGGCCTGG |
| | | | | | TGGCACCCAGCCAGTCCCTGTCTATCACATGTACCGTGTCC |
| | | | | | GGCTTCTCTCTGACCGATTACGGCGTGAACTGGGTGCGGC |
| | | | | | AGTCTCCTGGCAAGGGCCTGGAGTGGCTGGGCGTGATCTG |
| | | | | | GGGCGACGGCATCACAGATTATAATAGCGCCCTGAAGAGC |
| | | | | | AGGCTGTCCGTGACCAAGGATAACAGCAAGTCCCAGGTGT |
| | | | | | TTCTGAAGATGAACAGCCTGCAGAGCGGCGACTCCGCCCG |
| | | | | | CTACTATTGCGTGACAGGCCTGTTCGATTACTGGGGACAG |
| | | | | | GGCACCACACTGACCGTGTCCTCTCTCGAGTCTAGAGGGC |
| | | | | | CCTTCGAACAAAAACTCATCTCAGAAGAGGATCTGAATATG |
| | | | | | CATACCGGTCATCATCACCATCACCATTGA (SEQ ID |
| | | | | | NO: 98) |
| TMEF9LH- | TMEFF2 | VL-VH | NSGGGGS | H6 scFv | MAWVWTLLFLMAAAQSIQASYELTQPASVSGSPGQSITISCIG |
| H6 | | scFv | (SEQ ID NO: | | TSSDVGSYNLVSWYQQHPGKVPKLMIYEGSKRPSGVSNRFS |
| | | | 123) | | GSKSGNTASLTISGLQAEDEADYYCSSYAGSSTYVFGTGTKV |
| | | | | | TVLGGSEGKSSGSGSESKSTGGSQVQLQESGPGLVKPSETL |
| | | | | | SLTCTVSGVSISSYFWSWLRQPAGKGLQWIGRISTSGSTNHN |
| | | | | | PSLKSRVIMSVDTSKNQFSLKLSSVTAADTAVYYCVRDWTGF |
| | | | | | DYWGQGTLVTVSSNSGGGGSDAVVTQESALTTSPGETVTLT |
| | | | | | CRSSTGAVTTSNYASWVQEKPDHLFTGLIGGTNNRAPGVPAR |
| | | | | | FSGSLIGDKAALTITGAQTEDEAIYFCALWYSNHWVFGGGTKL |
| | | | | | TVLGGGGGSGGGGSGGGGSGGGGSDVQLQQSGPGLVAPS |
| | | | | | QSLSITCTVSGFSLTDYGVNWVRQSPGKGLEWLGVIWGDGIT |
| | | | | | DYNSALKSRLSVTKDNSKSQVFLKMNSLQSGDSARYYCVTGL |
| | | | | | FDYWGQGTTLTVSSLESRGPFEQKLISEEDLNMHTGHHHHHH |
| | | | | | (SEQ ID NO: 99) |
| | | | | | ATGGCCTGGGTGTGGACCCTGCTGTTCCTGATGGCAGCAG |
| | | | | | CACAGAGCATCCAGGCCTCCTACGAGCTGACACAGCCTGC |
| | | | | | ATCTGTGAGCGGCTCCCCAGGACAGTCTATCACCATCAGCT |
| | | | | | GCATCGGCACAAGCTCCGACGTGGGCTCCTACAACCTGGT |
| | | | | | GTCTTGGTATCAGCAGCACCCCGGCAAGGTGCCTAAGCTG |
| | | | | | ATGATCTATGAGGGCAGCAAGAGGCCAAGCGGCGTGTCCA |
| | | | | | ACAGATTCTCTGGCAGCAAGTCCGGCAATACCGCCTCCCT |
| | | | | | GACAATCTCTGGACTGCAGGCAGAGGACGAGGCAGATTAC |
| | | | | | TATTGCTCTAGCTACGCCGGCTCCTCTACCTACGTGTTCGG |
| | | | | | CACCGGCACAAAGGTGACAGTGCTGGGAGGCTCCGAGGG |
| | | | | | CAAGAGCTCCGGCTCTGGCAGCGAGTCCAAGTCTACCGGA |
| | | | | | GGCTCCCAGGTGCAGCTGCAGGAGAGCGGACCAGGACTG |
| | | | | | GTGAAGCCAAGCGAGACACTGTCCCTGACCTGTACAGTGT |
| | | | | | CTGGCGTGAGCATCTCTAGCTACTTTTGGAGCTGGCTGAG |
| | | | | | GCAGCCAGCAGGCAAGGGACTGCAGTGGATCGGCCGCAT |
| | | | | | CAGCACCTCCGGCTCTACAAACCACAATCCTTCCCTGAAGT |
| | | | | | CTAGAGTGATCATGTCTGTGGACACCAGCAAGAACCAGTTC |
| | | | | | TCCCTGAAGCTGTCCTCTGTGACCGCCGCCGATACAGCCG |
| | | | | | TGTACTATTGCGTGCGGGACTGGACCGGCTTTGATTATTGG |
| | | | | | GGCCAGGGCACCCTGGTGACAGTGAGCTCCAATAGCGGC |
| | | | | | GGCGGCGGATCCGATGCAGTGGTGACACAGGAGTCCGCC |
| | | | | | CTGACCACATCTCCAGGAGAGACCGTGACACTGACCTGTA |
| | | | | | GATCTAGCACCGGCGCCGTGACCACATCTAACTACGCCAG |
| | | | | | CTGGGTGCAGGAGAAGCCTGACCACCTGTTTACAGGCCTG |
| | | | | | ATCGGAGGCACCAACAATAGGGCACCAGGCGTGCCCGCAA |
| | | | | | GATTCTCCGGCTCTCTGATCGGCGACAAGGCCGCCCTGAC |
| | | | | | AATCACCGGAGCACAGACCGAGGATGAGGCCATCTACTTTT |
| | | | | | GCGCCCTGTGGTATTCCAATCACTGGGTCTTTGGAGGAGG |
| | | | | | CACAAAGCTGACCGTGCTGGGTGGAGGAGGAGGCAGCGG |
| | | | | | CGGAGGAGGCTCCGGAGGCGGCGGCTCTGGCGGCGGCG |
| | | | | | GCAGCGACGTGCAGCTGCAGCAGAGCGGACCAGGCCTGG |
| | | | | | TGGCACCCAGCCAGTCCCTGTCTATCACATGTACCGTGTCC |
| | | | | | GGCTTCTCTCTGACCGATTACGGCGTGAACTGGGTGCGGC |
| | | | | | AGTCTCCTGGCAAGGGCCTGGAGTGGCTGGGCGTGATCTG |
| | | | | | GGGCGACGGCATCACAGATTATAATAGCGCCCTGAAGAGC |
| | | | | | AGGCTGTCCGTGACCAAGGATAACAGCAAGTCCCAGGTGT |
| | | | | | TTCTGAAGATGAACAGCCTGCAGAGCGGCGACTCCGCCCG |
| | | | | | CTACTATTGCGTGACAGGCCTGTTCGATTACTGGGGACAG |
| | | | | | GGCACCACACTGACCGTGTCCTCTCTCGAGTCTAGAGGGC |
| | | | | | CCTTCGAACAAAAACTCATCTCAGAAGAGGATCTGAATATG |
| | | | | | CATACCGGTCATCATCACCATCACCATTGA (SEQ ID NO: |
| | | | | | 100) |
| KL2B359HL- | KLK2 | VH-VL | GGGGS | H6 scFv | MAWVWTLLFLMAAAQSIQAQVQLQESGPGLVKPSQTLSLTCT |
| H6 | | scFv | (SEQ ID NO: | | VSGNSITSDYAWNWIRQFPGKRLEWIGYISYSGSTTYNPSLKS |
| | | | 124) | | RVTISRDTSKNQFSLKLSSVTAADTAVYYCATGYYYGSGFWG |
| | | | | | QGTLVTVSSGGSEGKSSGSGSESKSTGGSEIVLTQSPATLSL |
| | | | | | SPGERATLSCRASESVEYFGTSLMHWYQQKPGQPPRLLIYAA |
| | | | | | SNVESGIPARFSGSGSGTDFTLTISSVEPEDFAVYFCQQTRKV |
| | | | | | PYTFGGGTKVEIKGGGGSDAVVTQESALTTSPGETVTLTCRS |
| | | | | | STGAVTTSNYASWVQEKPDHLFTGLIGGTNNRAPGVPARFSG |
| | | | | | SLIGDKAALTITGAQTEDEAIYFCALWYSNHWVFGGGTKLTVL |
| | | | | | GGGGGSGGGGSGGGGSGGGGSDVQLQQSGPGLVAPSQSL |
| | | | | | SITCTVSGFSLTDYGVNWVRQSPGKGLEWLGVIWGDGITDYN |
| | | | | | SALKSRLSVTKDNSKSQVFLKMNSLQSGDSARYYCVTGLFDY |
| | | | | | WGQGTTLTVSSGGGGSLESRGPFEQKLISEEDLNMHTGHHH |
| | | | | | HHH (SEQ ID NO: 101) |
| | | | | | ATGGCTTGGGTTTGGACCCTGCTGTTCCTGATGGCCGCTG |
| | | | | | CTCAGTCTATCCAGGCTCAGGTGCAGCTGCAGGAGTCCGG |
| | | | | | ACCAGGCCTGGTGAAGCCAAGCCAGACCCTGTCCCTGACC |
| | | | | | TGCACAGTGTCCGGCAACTCTATCACAAGCGACTATGCCTG |
| | | | | | GAATTGGATCAGGCAGTTCCCTGGCAAGCGCCTGGAGTGG |
| | | | | | ATCGGCTATATCTCTTACAGCGGCTCCACCACATACAACCC |
| | | | | | CTCCCTGAAGTCTCGGGTGACCATCAGCCGGGACACAAGC |
| | | | | | AAGAATCAGTTCAGCCTGAAGCTGAGCTCCGTGACCGCAG |
| | | | | | CAGATACAGCCGTGTACTATTGCGCCACCGGCTACTATTAC |
| | | | | | GGCTCCGGATTTTGGGGACAGGGCACCCTGGTGACAGTGT |
| | | | | | CTAGCGGAGGCAGCGAGGGCAAGTCCTCTGGCTCTGGCA |
| | | | | | GCGAGTCCAAGTCTACCGGCGGCAGCGAGATCGTGCTGAC |
| | | | | | CCAGTCCCCTGCCACACTGAGCCTGTCCCCAGGAGAGAGG |
| | | | | | GCCACCCTGTCTTGTAGAGCCTCTGAGAGCGTGGAGTATTT |
| | | | | | CGGCACAAGCCTGATGCACTGGTATCAGCAGAAGCCAGGC |
| | | | | | CAGCCCCCTAGGCTGCTGATCTATGCCGCCTCCAACGTGG |
| | | | | | AGTCTGGCATCCCCGCACGCTTCTCCGGCTCTGGCAGCGG |
| | | | | | CACCGACTTTACCCTGACAATCAGCTCCGTGGAGCCCGAG |
| | | | | | GATTTCGCCGTGTATTTTTGTCAGCAGACACGGAAGGTGCC |
| | | | | | TTACACCTTTGGCGGCGGCACAAAGGTGGAGATCAAGGGA |
| | | | | | GGAGGAGGATCCGACGCAGTGGTGACACAGGAGTCTGCC |
| | | | | | CTGACCACAAGCCCAGGCGAGACCGTGACACTGACCTGTA |
| | | | | | GGTCCTCTACCGGCGCCGTGACCACATCCAATTACGCCTC |
| | | | | | TTGGGTGCAGGAGAAGCCCGATCACCTGTTCACAGGCCTG |
| | | | | | ATCGGAGGCACCAACAATAGGGCACCAGGCGTGCCCGCCA |
| | | | | | GATTTTCTGGCAGCCTGATCGGCGACAAGGCCGCCCTGAC |
| | | | | | AATCACCGGAGCACAGACCGAGGATGAGGCCATCTACTTC |
| | | | | | TGCGCCCTGTGGTATAGCAACCACTGGGTGTTTGGCGGCG |
| | | | | | GCACAAAGCTGACCGTGCTGGGAGGAGGAGGAGGCTCCG |
| | | | | | GCGGAGGAGGCTCTGGCGGCGGCGGCAGCGGAGGCGGC |
| | | | | | GGCTCCGACGTGCAGCTGCAGCAGTCCGGACCTGGCCTG |
| | | | | | GTGGCACCATCCCAGTCTCTGAGCATCACATGTACCGTGA |
| | | | | | GCGGCTTTTCCCTGACCGATTACGGAGTGAACTGGGTGCG |
| | | | | | GCAGAGCCCAGGCAAGGGACTGGAGTGGCTGGGCGTGAT |
| | | | | | CTGGGGCGACGGCATCACAGATTATAATTCCGCCCTGAAG |
| | | | | | TCTAGGCTGAGCGTGACCAAGGATAACTCCAAGTCTCAGGT |
| | | | | | GTTCCTGAAGATGAACAGCCTGCAGTCTGGCGACAGCGCC |
| | | | | | CGCTACTATTGCGTGACAGGCCTGTTTGATTACTGGGGCCA |
| | | | | | GGGCACCACACTGACCGTGAGCTCCGGCGGCGGCGGCAG |
| | | | | | CCTCGAGTCTAGAGGGCCCTTCGAACAAAAACTCATCTCAG |
| | | | | | AAGAGGATCTGAATATGCATACCGGTCATCATCACCATCAC |
| | | | | | CATTGA (SEQ ID NO: 102) |
| KL2B359LH- | KLK2 | VL-VH | GGGGS | H6 scFv | MAWVWTLLFLMAAAQSIQAEIVLTQSPATLSLSPGERATLSCR |
| H6 | | scFv | (SEQ ID NO: | | ASESVEYFGTSLMHWYQQKPGQPPRLLIYAASNVESGIPARF |
| | | | 124) | | SGSGSGTDFTLTISSVEPEDFAVYFCQQTRKVPYTFGGGTKV |
| | | | | | EIKGGSEGKSSGSGSESKSTGGSQVQLQESGPGLVKPSQTL |
| | | | | | SLTCTVSGNSITSDYAWNWIRQFPGKRLEWIGYISYSGSTTYN |
| | | | | | PSLKSRVTISRDTSKNQFSLKLSSVTAADTAVYYCATGYYYGS |
| | | | | | GFWGQGTLVTVSSGGGGSDAVVTQESALTTSPGETVTLTCR |
| | | | | | SSTGAVTTSNYASWVQEKPDHLFTGLIGGTNNRAPGVPARFS |
| | | | | | GSLIGDKAALTITGAQTEDEAIYFCALWYSNHWVFGGGTKLTV |
| | | | | | LGGGGGSGGGGSGGGGSGGGGSDVQLQQSGPGLVAPSQS |
| | | | | | LSITCTVSGFSLTDYGVNWVRQSPGKGLEWLGVIWGDGITDY |
| | | | | | NSALKSRLSVTKDNSKSQVFLKMNSLQSGDSARYYCVTGLFD |
| | | | | | YWGQGTTLTVSSGGGGSLESRGPFEQKLISEEDLNMHTGHH |
| | | | | | HHHH (SEQ ID NO: 103) |
| | | | | | ATGGCTTGGGTTTGGACCCTGCTGTTCCTGATGGCCGCTG |
| | | | | | CTCAGTCTATCCAGGCTGAGATCGTGCTGACCCAGTCCCCT |
| | | | | | GCCACACTGTCTCTGAGCCCAGGAGAGAGGGCCACCCTGT |
| | | | | | CTTGCAGGGCATCCGAGTCTGTGGAGTATTTCGGCACAAG |
| | | | | | CCTGATGCACTGGTATCAGCAGAAGCCAGGCCAGCCCCCT |
| | | | | | AGGCTGCTGATCTATGCAGCAAGCAACGTGGAGTCCGGCA |
| | | | | | TCCCCGCACGCTTCAGCGGCTCCGGCTCTGGCACCGACTT |
| | | | | | TACCCTGACAATCAGCTCCGTGGAGCCCGAGGATTTCGCC |
| | | | | | GTGTATTTTTGCCAGCAGACACGGAAGGTGCCTTACACCTT |
| | | | | | TGGCGGCGGCACAAAGGTGGAGATCAAGGGAGGCTCCGA |
| | | | | | GGGCAAGTCTAGCGGCAGCGGCTCCGAGTCTAAGAGCACC |
| | | | | | GGAGGCAGCCAGGTGCAGCTGCAGGAGTCCGGACCAGGC |
| | | | | | CTGGTGAAGCCATCTCAGACCCTGAGCCTGACCTGTACAG |
| | | | | | TGTCCGGCAACTCTATCACAAGCGACTATGCCTGGAATTGG |
| | | | | | ATCAGACAGTTCCCTGGCAAGAGACTGGAGTGGATCGGCT |
| | | | | | ATATCTCCTACTCTGGCAGCACCACATACAACCCCTCCCTG |
| | | | | | AAGTCTCGGGTGACCATCTCCAGAGACACATCTAAGAATCA |
| | | | | | GTTCAGCCTGAAGCTGTCCTCTGTGACCGCCGCCGATACA |
| | | | | | GCCGTGTACTATTGTGCCACCGGCTACTATTACGGCTCCG |
| | | | | | GATTTTGGGGACAGGGCACCCTGGTGACAGTGAGCTCCGG |
| | | | | | AGGAGGAGGATCCGACGCAGTGGTGACACAGGAGTCTGC |
| | | | | | CCTGACCACAAGCCCAGGCGAGACCGTGACACTGACCTGT |
| | | | | | AGGTCCTCTACCGGCGCCGTGACCACATCCAATTACGCCT |
| | | | | | CTTGGGTGCAGGAGAAGCCCGATCACCTGTTCACAGGCCT |
| | | | | | GATCGGAGGCACCAACAATAGGGCACCAGGCGTGCCCGC |
| | | | | | CAGATTTTCTGGCAGCCTGATCGGCGACAAGGCCGCCCTG |
| | | | | | ACAATCACCGGAGCACAGACCGAGGATGAGGCCATCTACT |
| | | | | | TCTGCGCCCTGTGGTATAGCAACCACTGGGTGTTTGGCGG |
| | | | | | CGGCACAAAGCTGACCGTGCTGGGAGGAGGAGGAGGCTC |
| | | | | | CGGCGGAGGAGGCTCTGGCGGCGGCGGCAGCGGAGGCG |
| | | | | | GCGGCTCCGACGTGCAGCTGCAGCAGTCCGGACCTGGCC |
| | | | | | TGGTGGCACCATCCCAGTCTCTGAGCATCACATGTACCGTG |
| | | | | | AGCGGCTTTTCCCTGACCGATTACGGAGTGAACTGGGTGC |
| | | | | | GGCAGAGCCCAGGCAAGGGACTGGAGTGGCTGGGCGTGA |
| | | | | | TCTGGGGCGACGGCATCACAGATTATAATTCCGCCCTGAA |
| | | | | | GTCTAGGCTGAGCGTGACCAAGGATAACTCCAAGTCTCAG |
| | | | | | GTGTTCCTGAAGATGAACAGCCTGCAGTCTGGCGACAGCG |
| | | | | | CCCGCTACTATTGCGTGACAGGCCTGTTTGATTACTGGGGC |
| | | | | | CAGGGCACCACACTGACCGTGAGCTCCGGCGGCGGCGGC |
| | | | | | AGCCTCGAGTCTAGAGGGCCCTTCGAACAAAAACTCATCTC |
| | | | | | AGAAGAGGATCTGAATATGCATACCGGTCATCATCACCATC |
| | | | | | ACCATTGA (SEQ ID NO: 104) |
| H6- | KLK2 | H6 scFv | GGGGS | VH-VL | MAWVWTLLFLMAAAQSIQADAVVTQESALTTSPGETVTLTCR |
| KL2B359HL | | | (SEQ ID NO: | scFv | SSTGAVTTSNYASWVQEKPDHLFTGLIGGTNNRAPGVPARFS |
| | | | 124) | | GSLIGDKAALTITGAQTEDEAIYFCALWYSNHWVFGGGTKLTV |
| | | | | | LGGGGGSGGGGSGGGGSGGGGSDVQLQQSGPGLVAPSQS |
| | | | | | LSITCTVSGFSLTDYGVNWVRQSPGKGLEWLGVIWGDGITDY |
| | | | | | NSALKSRLSVTKDNSKSQVFLKMNSLQSGDSARYYCVTGLFD |
| | | | | | YWGQGTTLTVSSGGGGSQVQLQESGPGLVKPSQTLSLTCTV |
| | | | | | SGNSITSDYAWNWIRQFPGKRLEWIGYISYSGSTTYNPSLKSR |
| | | | | | VTISRDTSKNQFSLKLSSVTAADTAVYYCATGYYYGSGFWGQ |
| | | | | | GTLVTVSSGGSEGKSSGSGSESKSTGGSEIVLTQSPATLSLSP |
| | | | | | GERATLSCRASESVEYFGTSLMHWYQQKPGQPPRLLIYAASN |
| | | | | | VESGIPARFSGSGSGTDFTLTISSVEPEDFAVYFCQQTRKVPY |
| | | | | | TFGGGTKVEIKLESRGPFEQKLISEEDLNMHTGHHHHHH |
| | | | | | (SEQ ID NO: 105) |
| | | | | | ATGGCTTGGGTTTGGACCCTGCTGTTCCTGATGGCCGCTG |
| | | | | | CTCAGTCTATCCAGGCTGATGCAGTGGTGACACAGGAGAG |
| | | | | | CGCCCTGACCACATCCCCAGGAGAGACCGTGACACTGACC |
| | | | | | TGCAGGAGCTCCACCGGAGCAGTGACCACATCCAACTACG |
| | | | | | CCTCTTGGGTGCAGGAGAAGCCCGATCACCTGTTCACAGG |
| | | | | | CCTGATCGGCGGCACCAACAATAGGGCACCAGGCGTGCCC |
| | | | | | GCACGCTTTTCTGGCAGCCTGATCGGCGACAAGGCCGCCC |
| | | | | | TGACAATCACCGGAGCACAGACAGAGGATGAGGCCATCTA |
| | | | | | CTTCTGCGCCCTGTGGTATAGCAATCACTGGGTGTTTGGCG |
| | | | | | GCGGCACAAAGCTGACCGTGCTGGGAGGAGGAGGAGGCT |
| | | | | | CTGGAGGAGGAGGCAGCGGCGGCGGCGGCTCCGGCGGC |
| | | | | | GGCGGCTCTGACGTGCAGCTGCAGCAGTCCGGACCTGGC |
| | | | | | CTGGTGGCACCATCCCAGTCTCTGAGCATCACATGTACCGT |
| | | | | | GAGCGGCTTCTCCCTGACCGATTACGGAGTGAACTGGGTG |
| | | | | | CGGCAGTCCCCTGGCAAGGGACTGGAGTGGCTGGGCGTG |
| | | | | | ATCTGGGGCGACGGCATCACAGATTATAATTCTGCCCTGAA |
| | | | | | GTCTAGGCTGAGCGTGACCAAGGACAACTCCAAGTCTCAG |
| | | | | | GTGTTCCTGAAGATGAACAGCCTGCAGTCTGGCGACAGCG |
| | | | | | CCCGCTACTATTGCGTGACAGGCCTGTTTGATTACTGGGGC |
| | | | | | CAGGGCACCACACTGACCGTGTCTTCGGGAGGAGGAGGAT |
| | | | | | CCCAGGTGCAGCTGCAGGAGTCCGGACCAGGCCTGGTGA |
| | | | | | AGCCAAGCCAGACCCTGTCCCTGACCTGCACAGTGTCCGG |
| | | | | | CAACTCTATCACAAGCGACTATGCCTGGAATTGGATCAGGC |
| | | | | | AGTTCCCTGGCAAGCGCCTGGAGTGGATCGGCTATATCTC |
| | | | | | TTACAGCGGCTCCACCACATACAACCCCTCCCTGAAGTCTC |
| | | | | | GGGTGACCATCAGCCGGGACACAAGCAAGAATCAGTTCAG |
| | | | | | CCTGAAGCTGAGCTCCGTGACCGCAGCAGATACAGCCGTG |
| | | | | | TACTATTGCGCCACCGGCTACTATTACGGCTCCGGATTTTG |
| | | | | | GGGACAGGGCACCCTGGTGACAGTGTCTAGCGGAGGCAG |
| | | | | | CGAGGGCAAGTCCTCTGGCTCTGGCAGCGAGTCCAAGTCT |
| | | | | | ACCGGCGGCAGCGAGATCGTGCTGACCCAGTCCCCTGCCA |
| | | | | | CACTGAGCCTGTCCCCAGGAGAGAGGGCCACCCTGTCTTG |
| | | | | | TAGAGCCTCTGAGAGCGTGGAGTATTTCGGCACAAGCCTG |
| | | | | | ATGCACTGGTATCAGCAGAAGCCAGGCCAGCCCCCTAGGC |
| | | | | | TGCTGATCTATGCCGCCTCCAACGTGGAGTCTGGCATCCC |
| | | | | | CGCACGCTTCTCCGGCTCTGGCAGCGGCACCGACTTTACC |
| | | | | | CTGACAATCAGCTCCGTGGAGCCCGAGGATTTCGCCGTGT |
| | | | | | ATTTTTGTCAGCAGACACGGAAGGTGCCTTACACCTTTGGC |
| | | | | | GGCGGCACAAAGGTGGAGATCAAGCTCGAGTCTAGAGGGC |
| | | | | | CCTTCGAACAAAAACTCATCTCAGAAGAGGATCTGAATATG |
| | | | | | CATACCGGTCATCATCACCATCACCATTGA (SEQ ID NO: |
| | | | | | 106) |
| H6- | KLK2 | H6 scFv | GGGGS | VL-VH | MAWVWTLLFLMAAAQSIQADAVVTQESALTTSPGETVTLTCR |
| KL2B359LH | | | (SEQ ID NO: | scFv | SSTGAVTTSNYASWVQEKPDHLFTGLIGGTNNRAPGVPARFS |
| | | | 124) | | GSLIGDKAALTITGAQTEDEAIYFCALWYSNHWVFGGGTKLTV |
| | | | | | LGGGGGSGGGGSGGGGSGGGGSDVQLQQSGPGLVAPSQS |
| | | | | | LSITCTVSGFSLTDYGVNWVRQSPGKGLEWLGVIWGDGITDY |
| | | | | | NSALKSRLSVTKDNSKSQVFLKMNSLQSGDSARYYCVTGLFD |
| | | | | | YWGQGTTLTVSSGGGGSEIVLTQSPATLSLSPGERATLSCRA |
| | | | | | SESVEYFGTSLMHWYQQKPGQPPRLLIYAASNVESGIPARFS |
| | | | | | GSGSGTDFTLTISSVEPEDFAVYFCQQTRKVPYTFGGGTKVEI |
| | | | | | KGGSEGKSSGSGSESKSTGGSQVQLQESGPGLVKPSQTLSL |
| | | | | | TCTVSGNSITSDYAWNWIRQFPGKRLEWIGYISYSGSTTYNPS |
| | | | | | LKSRVTISRDTSKNQFSLKLSSVTAADTAVYYCATGYYYGSGF |
| | | | | | WGQGTLVTVSSLESRGPFEQKLISEEDLNMHTGHHHHHH |
| | | | | | (SEQ ID NO: 107) |
| | | | | | ATGGCTTGGGTTTGGACCCTGCTGTTCCTGATGGCCGCTG |
| | | | | | CTCAGTCTATCCAGGCTGATGCAGTGGTGACACAGGAGAG |
| | | | | | CGCCCTGACCACATCCCCAGGAGAGACCGTGACACTGACC |
| | | | | | TGCAGGAGCTCCACCGGAGCAGTGACCACATCCAACTACG |
| | | | | | CCTCTTGGGTGCAGGAGAAGCCCGATCACCTGTTCACAGG |
| | | | | | CCTGATCGGCGGCACCAACAATAGGGCACCAGGCGTGCCC |
| | | | | | GCACGCTTTTCTGGCAGCCTGATCGGCGACAAGGCCGCCC |
| | | | | | TGACAATCACCGGAGCACAGACAGAGGATGAGGCCATCTA |
| | | | | | CTTCTGCGCCCTGTGGTATAGCAATCACTGGGTGTTTGGCG |
| | | | | | GCGGCACAAAGCTGACCGTGCTGGGAGGAGGAGGAGGCT |
| | | | | | CTGGAGGAGGAGGCAGCGGCGGCGGCGGCTCCGGCGGC |
| | | | | | GGCGGCTCTGACGTGCAGCTGCAGCAGTCCGGACCTGGC |
| | | | | | CTGGTGGCACCATCCCAGTCTCTGAGCATCACATGTACCGT |
| | | | | | GAGCGGCTTCTCCCTGACCGATTACGGAGTGAACTGGGTG |
| | | | | | CGGCAGTCCCCTGGCAAGGGACTGGAGTGGCTGGGCGTG |
| | | | | | ATCTGGGGCGACGGCATCACAGATTATAATTCTGCCCTGAA |
| | | | | | GTCTAGGCTGAGCGTGACCAAGGACAACTCCAAGTCTCAG |
| | | | | | GTGTTCCTGAAGATGAACAGCCTGCAGTCTGGCGACAGCG |
| | | | | | CCCGCTACTATTGCGTGACAGGCCTGTTTGATTACTGGGGC |
| | | | | | CAGGGCACCACACTGACCGTGTCTTCGGGAGGAGGAGGAT |
| | | | | | CCGAGATCGTGCTGACCCAGTCCCCTGCCACACTGTCTCT |
| | | | | | GAGCCCAGGAGAGAGGGCCACCCTGTCTTGCAGGGCATC |
| | | | | | CGAGTCTGTGGAGTATTTCGGCACAAGCCTGATGCACTGG |
| | | | | | TATCAGCAGAAGCCAGGCCAGCCCCCTAGGCTGCTGATCT |
| | | | | | ATGCAGCAAGCAACGTGGAGTCCGGCATCCCCGCACGCTT |
| | | | | | CAGCGGCTCCGGCTCTGGCACCGACTTTACCCTGACAATC |
| | | | | | AGCTCCGTGGAGCCCGAGGATTTCGCCGTGTATTTTTGCCA |
| | | | | | GCAGACACGGAAGGTGCCTTACACCTTTGGCGGCGGCACA |
| | | | | | AAGGTGGAGATCAAGGGAGGCTCCGAGGGCAAGTCTAGC |
| | | | | | GGCAGCGGCTCCGAGTCTAAGAGCACCGGAGGCAGCCAG |
| | | | | | GTGCAGCTGCAGGAGTCCGGACCAGGCCTGGTGAAGCCAT |
| | | | | | CTCAGACCCTGAGCCTGACCTGTACAGTGTCCGGCAACTC |
| | | | | | TATCACAAGCGACTATGCCTGGAATTGGATCAGACAGTTCC |
| | | | | | CTGGCAAGAGACTGGAGTGGATCGGCTATATCTCCTACTCT |
| | | | | | GGCAGCACCACATACAACCCCTCCCTGAAGTCTCGGGTGA |
| | | | | | CCATCTCCAGAGACACATCTAAGAATCAGTTCAGCCTGAAG |
| | | | | | CTGTCCTCTGTGACCGCCGCCGATACAGCCGTGTACTATTG |
| | | | | | TGCCACCGGCTACTATTACGGCTCCGGATTTTGGGGACAG |
| | | | | | GGCACCCTGGTGACAGTGAGCTCCCTCGAGTCTAGAGGGC |
| | | | | | CCTTCGAACAAAAACTCATCTCAGAAGAGGATCTGAATATG |
| | | | | | CATACCGGTCATCATCACCATCACCATTGA (SEQ ID NO: |
| | | | | | 108) |
| H6W-H6 | ROR1 | DARPin | NSGGGGSTG | H6 scFv | METDTLLLWVLLLWVPGSTGDGSDLGKKLLEAARAGQDDEVR |
| | | | (SEQ ID NO: | | ILMANGADVNASDRYGRTPLHLAAFNGHLEIVEVLLKNGADVN |
| | | | 125) | | AKDKIGNTPLHLAANHGHLEIVEVLLKYGAVVNATDWLGVTPL |
| | | | | | HLAAVFGHLEIVEVLLKYGADVNAQDKFGKTAFDISIDNGNEDL |
| | | | | | AEILQKLNSGGGGSTGDAVVTQESALTTSPGETVTLTCRSSTG |
| | | | | | AVTTSNYASWVQEKPDHLFTGLIGGTNNRAPGVPARFSGSLI |
| | | | | | GDKAALTITGAQTEDEAIYFCALWYSNHWVFGGGTKLTVLGG |
| | | | | | GGGSGGGGSGGGGSGGGGSDVQLQQSGPGLVAPSQSLSIT |
| | | | | | CTVSGFSLTDYGVNWVRQSPGKGLEWLGVIWGDGITDYNSA |
| | | | | | LKSRLSVTKDNSKSQVFLKMNSLQSGDSARYYCVTGLFDYW |
| | | | | | GQGTTLTVSSGGGGSLESRGPFEQKLISEEDLNMHTGHHHH |
| | | | | | HH (SEQ ID NO: 109) |
| | | | | | ATGGAGACCGACACACTGCTGCTGTGGGTGCTGCTGCTGT |
| | | | | | GGGTGCCCGGCTCTACCGGCGACGGCAGCGATCTGGGCA |
| | | | | | AGAAGCTGCTGGAGGCAGCCAGAGCCGGACAGGACGATG |
| | | | | | AGGTGAGAATCCTGATGGCCAACGGCGCCGACGTGAATGC |
| | | | | | CAGCGATCGGTACGGCAGAACACCACTGCACCTGGCAGCC |
| | | | | | TTCAACGGACACCTGGAGATCGTGGAGGTGCTGCTGAAGA |
| | | | | | ATGGAGCCGACGTGAATGCCAAGGATAAGATCGGCAACAC |
| | | | | | CCCTCTGCACCTGGCAGCAAATCATGGCCACCTGGAGATT |
| | | | | | GTCGAGGTGCTGCTGAAGTACGGCGCCGTGGTGAATGCCA |
| | | | | | CAGACTGGCTGGGAGTGACCCCCCTGCACCTGGCCGCCG |
| | | | | | TGTTTGGCCACCTGGAGATCGTCGAAGTCCTGCTGAAGTAT |
| | | | | | GGCGCCGACGTGAACGCCCAGGATAAGTTCGGCAAGACAG |
| | | | | | CCTTTGACATCTCCATCGATAACGGCAATGAGGACCTGGCC |
| | | | | | GAGATCCTGCAGAAGCTGAATTCTGGAGGAGGAGGCTCTA |
| | | | | | CAGGCGATGCAGTGGTGACCCAGGAGAGCGCCCTGACCA |
| | | | | | CATCCCCTGGAGAGACCGTGACACTGACCTGCCGGAGCTC |
| | | | | | CACCGGAGCAGTGACCACAAGCAACTATGCCTCCTGGGTG |
| | | | | | CAGGAGAAGCCAGATCACCTGTTCACAGGCCTGATCGGAG |
| | | | | | GCACCAACAATAGGGCACCAGGCGTGCCTGCACGCTTTTC |
| | | | | | CGGCTCTCTGATCGGCGACAAGGCCGCCCTGACAATCACC |
| | | | | | GGAGCACAGACCGAGGATGAGGCCATCTACTTCTGCGCCC |
| | | | | | TGTGGTATTCTAATCACTGGGTGTTTGGCGGCGGCACAAAG |
| | | | | | CTGACCGTGCTGGGAGGAGGAGGAGGCTCTGGAGGAGGA |
| | | | | | GGCAGCGGCGGCGGCGGCTCCGGCGGCGGCGGCTCTGA |
| | | | | | CGTGCAGCTGCAGCAGTCCGGACCAGGCCTGGTGGCACC |
| | | | | | CAGCCAGTCCCTGTCTATCACATGTACCGTGTCTGGCTTCA |
| | | | | | GCCTGACCGATTACGGAGTGAACTGGGTGCGGCAGAGCCC |
| | | | | | TGGCAAGGGACTGGAGTGGCTGGGCGTGATCTGGGGCGA |
| | | | | | CGGCATCACAGATTATAATTCCGCCCTGAAGTCCAGGCTGT |
| | | | | | CTGTGACCAAGGATAACAGCAAGTCCCAGGTGTTCCTGAA |
| | | | | | GATGAATAGCCTGCAGTCCGGCGACTCTGCCCGCTACTATT |
| | | | | | GCGTGACAGGCCTGTTTGATTACTGGGGCCAGGGCACCAC |
| | | | | | ACTGACCGTGTCTAGCGGCGGCGGCGGCAGCCTCGAGTC |
| | | | | | TAGAGGGCCCTTCGAACAAAAACTCATCTCAGAAGAGGATC |
| | | | | | TGAATATGCATACCGGTCATCATCACCATCACCATTGA |
| | | | | | (SEQ ID NO: 110) |
| B588LH- | PMSA | VL-VH | GGGGSGGG | EE12RR | METDTLLLWVLLLWVPGSTGQSVLTQPPSVSGAPGQRVTISC |
| EE12RR345L | | scFv | GSGGGGS | 345L | TGSSSNIGAGYDVHWYQQLPGTAPKLLIYGNTNRPSGVPDRF |
| | | | (SEQ ID NO: | | SGSKSGTSASLAITGLQAEDEADYYCQSYDSSLSGTPYVVFG |
| | | | 126) | | GGTKLTVLGGSEGKSSGSGSESKSTGGSEVQLVESGGGLVQ |
| | | | | | PGGSLRLSCAASGFTFSFYNMNWVRQAPGKGLEWISYISTSS |
| | | | | | STIYYADSVKGRFTISRDNAKNSLYLQMNSLRDEDTAVYYCAR |
| | | | | | EGSYYDSSGYPYYYYDMDVWGQGTTVTVSSGGGGGGGGS |
| | | | | | GGGGSLEIEAAFLERENTALETRVAELRQRVQRLRNRVSQYR |
| | | | | | TRYGPLGGGGSLESRGPFEQKLISEEDLNMHTGHHHHHH |
| | | | | | (SEQ ID NO: 111) |
| | | | | | ATGGAGACAGACACACTGCTGCTGTGGGTGCTGCTGCTGT |
| | | | | | GGGTACCAGGCAGCACAGGCCAGTCTGTGCTGACCCAGCC |
| | | | | | ACCTAGCGTGTCCGGAGCACCAGGCCAGCGGGTGACAATC |
| | | | | | TCCTGCACCGGCAGCTCCTCTAACATCGGCGCCGGCTACG |
| | | | | | ACGTGCACTGGTATCAGCAGCTGCCTGGCACAGCCCCAAA |
| | | | | | GCTGCTGATCTACGGCAACACCAATAGGCCCAGCGGCGTG |
| | | | | | CCTGATCGCTTTTCTGGCAGCAAGTCCGGCACATCTGCCA |
| | | | | | GCCTGGCAATCACCGGACTGCAGGCAGAGGACGAGGCCG |
| | | | | | ATTACTATTGCCAGTCTTACGACAGCTCCCTGAGCGGCACA |
| | | | | | CCTTATGTGGTGTTCGGAGGAGGCACAAAGCTGACCGTGC |
| | | | | | TGGGAGGCAGCGAGGGCAAGTCTAGCGGCTCCGGCTCTG |
| | | | | | AGAGCAAGTCCACCGGAGGCAGCGAGGTGCAGCTGGTGG |
| | | | | | AGTCCGGAGGAGGCCTGGTGCAGCCAGGAGGCAGCCTGC |
| | | | | | GGCTGTCCTGTGCCGCCTCTGGCTTCACCTTTTCCTTCTAC |
| | | | | | AACATGAATTGGGTGAGACAGGCACCTGGCAAGGGCCTGG |
| | | | | | AGTGGATCAGCTATATCTCCACATCCTCTAGCACCATCTAC |
| | | | | | TATGCCGACAGCGTGAAGGGCCGGTTTACAATCAGCCGGG |
| | | | | | ACAACGCCAAGAATAGCCTGTACCTGCAGATGAACAGCCT |
| | | | | | GAGGGACGAGGATACCGCCGTGTACTATTGCGCCCGCGAG |
| | | | | | GGCTCCTACTATGACTCCTCTGGCTATCCATACTATTACTAT |
| | | | | | GACATGGACGTGTGGGGCCAGGGCACCACAGTGACAGTG |
| | | | | | AGCTCCGGAGGAGGAGGATCCGGCGGCGGAGGATCTGGC |
| | | | | | GGCGGAGGCAGCCTGGAAATCGAGGCCGCCTTCCTGGAA |
| | | | | | CGGGAAAACACCGCCCTGGAGACAAGAGTCGCCGAGCTGA |
| | | | | | GACAGCGGGTGCAGAGACTGCGGAATAGAGTGTCCCAATA |
| | | | | | CCGCACCAGATACGGCCCTCTGGGGGGGGGGGGCAGCCT |
| | | | | | CGAGTCTAGAGGGCCCTTCGAACAAAAACTCATCTCAGAAG |
| | | | | | AGGATCTGAATATGCATACCGGTCATCATCACCATCACCAT |
| | | | | | TGA (SEQ ID NO: 112) |
| KL2B359LH- | KLK2 | VL-VH | GGGGSGGG | EE12RR | MAWVWTLLFLMAAAQSIQAEIVLTQSPATLSLSPGERATLSCR |
| EE12RR345L | | scFv | GSGGGGS | 345L | ASESVEYFGTSLMHWYQQKPGQPPRLLIYAASNVESGIPARF |
| | | | (SEQ ID NO: | | SGSGSGTDFTLTISSVEPEDFAVYFCQQTRKVPYTFGGGTKV |
| | | | 126) | | EIKGGSEGKSSGSGSESKSTGGSQVQLQESGPGLVKPSQTL |
| | | | | | SLTCTVSGNSITSDYAWNWIRQFPGKRLEWIGYISYSGSTTYN |
| | | | | | PSLKSRVTISRDTSKNQFSLKLSSVTAADTAVYYCATGYYYGS |
| | | | | | GFWGQGTLVTVSSGGGGSGGGGSGGGGSLEIEAAFLERENT |
| | | | | | ALETRVAELRQRVQRLRNRVSQYRTRYGPLGGGGSLESRGP |
| | | | | | FEQKLISEEDLNMHTGHHHHHH (SEQ ID NO: 113) |
| | | | | | ATGGCTTGGGTTTGGACCCTGCTGTTCCTGATGGCCGCTG |
| | | | | | CTCAGTCTATCCAGGCTGAGATCGTGCTGACCCAGTCCCCT |
| | | | | | GCCACACTGTCTCTGAGCCCAGGAGAGAGGGCCACCCTGT |
| | | | | | CTTGCAGGGCATCCGAGTCTGTGGAGTATTTCGGCACAAG |
| | | | | | CCTGATGCACTGGTATCAGCAGAAGCCAGGCCAGCCCCCT |
| | | | | | AGGCTGCTGATCTATGCAGCAAGCAACGTGGAGTCCGGCA |
| | | | | | TCCCCGCACGCTTCAGCGGCTCCGGCTCTGGCACCGACTT |
| | | | | | TACCCTGACAATCAGCTCCGTGGAGCCCGAGGATTTCGCC |
| | | | | | GTGTATTTTTGCCAGCAGACACGGAAGGTGCCTTACACCTT |
| | | | | | TGGCGGCGGCACAAAGGTGGAGATCAAGGGAGGCTCCGA |
| | | | | | GGGCAAGTCTAGCGGCAGCGGCTCCGAGTCTAAGAGCACC |
| | | | | | GGAGGCAGCCAGGTGCAGCTGCAGGAGTCCGGACCAGGC |
| | | | | | CTGGTGAAGCCATCTCAGACCCTGAGCCTGACCTGTACAG |
| | | | | | TGTCCGGCAACTCTATCACAAGCGACTATGCCTGGAATTGG |
| | | | | | ATCAGACAGTTCCCTGGCAAGAGACTGGAGTGGATCGGCT |
| | | | | | ATATCTCCTACTCTGGCAGCACCACATACAACCCCTCCCTG |
| | | | | | AAGTCTCGGGTGACCATCTCCAGAGACACATCTAAGAATCA |
| | | | | | GTTCAGCCTGAAGCTGTCCTCTGTGACCGCCGCCGATACA |
| | | | | | GCCGTGTACTATTGTGCCACCGGCTACTATTACGGCTCCG |
| | | | | | GATTTTGGGGACAGGGCACCCTGGTGACAGTGAGCTCCGG |
| | | | | | AGGAGGAGGATCCGGCGGCGGAGGATCTGGCGGCGGAG |
| | | | | | GCAGCCTGGAAATCGAGGCCGCCTTCCTGGAACGGGAAAA |
| | | | | | CACCGCCCTGGAGACAAGAGTCGCCGAGCTGAGACAGCG |
| | | | | | GGTGCAGAGACTGCGGAATAGAGTGTCCCAATACCGCACC |
| | | | | | AGATACGGCCCTCTGGGGGGGGGGGGCAGCCTCGAGTCT |
| | | | | | AGAGGGCCCTTCGAACAAAAACTCATCTCAGAAGAGGATCT |
| | | | | | GAATATGCATACCGGTCATCATCACCATCACCATTGA (SEQ |
| | | | | | ID NO: 114) |
| TMEF9LH- | TMEFF2 | VL-VH | NSGGGGSG | EE12R345L | MAWVWTLLFLMAAAQSIQASYELTQPASVSGSPGQSITISCIG |
| EE12RR345L | | scFv | GGGSGGGG | | TSSDVGSYNLVSWYQQHPGKVPKLMIYEGSKRPSGVSNRFS |
| | | | S (SEQ ID | | GSKSGNTASLTISGLQAEDEADYYCSSYAGSSTYVFGTGTKV |
| | | | NO: 127) | | TVLGGSEGKSSGSGSESKSTGGSQVQLQESGPGLVKPSETL |
| | | | | | SLTCTVSGVSISSYFWSWLRQPAGKGLQWIGRISTSGSTNHN |
| | | | | | PSLKSRVIMSVDTSKNQFSLKLSSVTAADTAVYYCVRDWTGF |
| | | | | | DYWGQGTLVTVSSNSGGGGSGGGGSGGGGSLEIEAAFLERE |
| | | | | | NTALETRVAELRQRVQRLRNRVSQYRTRYGPLGGGGSLESR |
| | | | | | GPFEQKLISEEDLNMHTGHHHHHH (SEQ ID NO: 115) |
| | | | | | ATGGCCTGGGTGTGGACCCTGCTGTTCCTGATGGCAGCAG |
| | | | | | CACAGAGCATCCAGGCCTCCTACGAGCTGACACAGCCTGC |
| | | | | | ATCTGTGAGCGGCTCCCCAGGACAGTCTATCACCATCAGCT |
| | | | | | GCATCGGCACAAGCTCCGACGTGGGCTCCTACAACCTGGT |
| | | | | | GTCTTGGTATCAGCAGCACCCCGGCAAGGTGCCTAAGCTG |
| | | | | | ATGATCTATGAGGGCAGCAAGAGGCCAAGCGGCGTGTCCA |
| | | | | | ACAGATTCTCTGGCAGCAAGTCCGGCAATACCGCCTCCCT |
| | | | | | GACAATCTCTGGACTGCAGGCAGAGGACGAGGCAGATTAC |
| | | | | | TATTGCTCTAGCTACGCCGGCTCCTCTACCTACGTGTTCGG |
| | | | | | CACCGGCACAAAGGTGACAGTGCTGGGAGGCTCCGAGGG |
| | | | | | CAAGAGCTCCGGCTCTGGCAGCGAGTCCAAGTCTACCGGA |
| | | | | | GGCTCCCAGGTGCAGCTGCAGGAGAGCGGACCAGGACTG |
| | | | | | GTGAAGCCAAGCGAGACACTGTCCCTGACCTGTACAGTGT |
| | | | | | CTGGCGTGAGCATCTCTAGCTACTTTTGGAGCTGGCTGAG |
| | | | | | GCAGCCAGCAGGCAAGGGACTGCAGTGGATCGGCCGCAT |
| | | | | | CAGCACCTCCGGCTCTACAAACCACAATCCTTCCCTGAAGT |
| | | | | | CTAGAGTGATCATGTCTGTGGACACCAGCAAGAACCAGTTC |
| | | | | | TCCCTGAAGCTGTCCTCTGTGACCGCCGCCGATACAGCCG |
| | | | | | TGTACTATTGCGTGCGGGACTGGACCGGCTTTGATTATTGG |
| | | | | | GGCCAGGGCACCCTGGTGACAGTGAGCTCCAATAGCGGC |
| | | | | | GGCGGCGGATCCGGCGGCGGAGGATCTGGCGGCGGAGG |
| | | | | | CAGCCTGGAAATCGAGGCCGCCTTCCTGGAACGGGAAAAC |
| | | | | | ACCGCCCTGGAGACAAGAGTCGCCGAGCTGAGACAGCGG |
| | | | | | GTGCAGAGACTGCGGAATAGAGTGTCCCAATACCGCACCA |
| | | | | | GATACGGCCCTCTGGGGGGGGGGGGCAGCCTCGAGTCTA |
| | | | | | GAGGGCCCTTCGAACAAAAACTCATCTCAGAAGAGGATCTG |
| | | | | | AATATGCATACCGGTCATCATCACCATCACCATTGA (SEQ ID |
| | | | | | NO: 116) |
| B588LH- | PMSA | VL-VH | GGGGS (SEQ | La5B9 | METDTLLLWVLLLWVPGSTGQSVLTQPPSVSGAPGQRVTISC |
| 5B9HL | | scFv | ID NO: 124) | scFv | TGSSSNIGAGYDVHWYQQLPGTAPKLLIYGNTNRPSGVPDRF |
| | | | | | SGSKSGTSASLAITGLQAEDEADYYCQSYDSSLSGTPYVVFG |
| | | | | | GGTKLTVLGGSEGKSSGSGSESKSTGGSEVQLVESGGGLVQ |
| | | | | | PGGSLRLSCAASGFTFSFYNMNWVRQAPGKGLEWISYISTSS |
| | | | | | STIYYADSVKGRFTISRDNAKNSLYLQMNSLRDEDTAVYYCAR |
| | | | | | EGSYYDSSGYPYYYYDMDVWGQGTTVTVSSGGGGSQVQLV |
| | | | | | QSGAEVKKPGASVKVSCKASGYTFTHYYIYWVRQAPGQGLE |
| | | | | | WMGGVNPSNGGTHFNEKFKSRVTMTRDTSISTAYMELSRLR |
| | | | | | SDDTAVYYCARSEYDYGLGFAYWGQGTLVTVSSGGSEGKSS |
| | | | | | GSGSESKSTGGSDIVMTQSPDSLAVSLGERATINCKSSQSLLN |
| | | | | | SRTPKNYLAWYQQKPGQPPKLLIYWASTRKSGVPDRFSGSG |
| | | | | | SGTDFTLTISSLQAEDVAVYYCKQSYNLLTFGGGTKVEIKLESR |
| | | | | | GPFEQKLISEEDLNMHTGHHHHHH (SEQ ID NO: 117) |
| | | | | | ATGGAGACAGACACACTGCTGCTGTGGGTGCTGCTGCTGT |
| | | | | | GGGTACCAGGCAGCACAGGCCAGTCTGTGCTGACCCAGCC |
| | | | | | ACCTAGCGTGTCCGGAGCACCAGGCCAGCGGGTGACAATC |
| | | | | | TCCTGCACCGGCAGCTCCTCTAACATCGGCGCCGGCTACG |
| | | | | | ACGTGCACTGGTATCAGCAGCTGCCTGGCACAGCCCCAAA |
| | | | | | GCTGCTGATCTACGGCAACACCAATAGGCCCAGCGGCGTG |
| | | | | | CCTGATCGCTTTTCTGGCAGCAAGTCCGGCACATCTGCCA |
| | | | | | GCCTGGCAATCACCGGACTGCAGGCAGAGGACGAGGCCG |
| | | | | | ATTACTATTGCCAGTCTTACGACAGCTCCCTGAGCGGCACA |
| | | | | | CCTTATGTGGTGTTCGGAGGAGGCACAAAGCTGACCGTGC |
| | | | | | TGGGAGGCAGCGAGGGCAAGTCTAGCGGCTCCGGCTCTG |
| | | | | | AGAGCAAGTCCACCGGAGGCAGCGAGGTGCAGCTGGTGG |
| | | | | | AGTCCGGAGGAGGCCTGGTGCAGCCAGGAGGCAGCCTGC |
| | | | | | GGCTGTCCTGTGCCGCCTCTGGCTTCACCTTTTCCTTCTAC |
| | | | | | AACATGAATTGGGTGAGACAGGCACCTGGCAAGGGCCTGG |
| | | | | | AGTGGATCAGCTATATCTCCACATCCTCTAGCACCATCTAC |
| | | | | | TATGCCGACAGCGTGAAGGGCCGGTTTACAATCAGCCGGG |
| | | | | | ACAACGCCAAGAATAGCCTGTACCTGCAGATGAACAGCCT |
| | | | | | GAGGGACGAGGATACCGCCGTGTACTATTGCGCCCGCGAG |
| | | | | | GGCTCCTACTATGACTCCTCTGGCTATCCATACTATTACTAT |
| | | | | | GACATGGACGTGTGGGGCCAGGGCACCACAGTGACAGTG |
| | | | | | AGCTCCGGAGGAGGAGGATCCCAAGTGCAACTGGTCCAGT |
| | | | | | CAGGTGCTGAGGTGAAAAAACCCGGAGCCAGTGTCAAAGT |
| | | | | | AAGCTGCAAGGCCTCTGGGTATACTTTCACCCATTACTATA |
| | | | | | TATACTGGGTTCGTCAAGCTCCAGGTCAGGGGCTTGAGTG |
| | | | | | GATGGGTGGAGTCAACCCTTCGAACGGTGGCACTCACTTC |
| | | | | | AATGAAAAGTTTAAAAGCCGCGTAACCATGACGCGAGATAC |
| | | | | | TTCCATTTCCACAGCTTATATGGAACTTAGTAGGTTACGCAG |
| | | | | | TGATGACACGGCCGTTTATTACTGTGCTAGAAGTGAATATG |
| | | | | | ATTATGGGTTGGGTTTCGCTTACTGGGGCCAGGGAACCCT |
| | | | | | CGTCACCGTGTCCAGTGGAGGCAGCGAGGGCAAGTCTAGC |
| | | | | | GGCTCCGGCTCTGAGAGCAAGTCCACCGGAGGCAGCGAC |
| | | | | | ATTGTTATGACGCAGAGCCCTGATTCACTCGCAGTGTCCCT |
| | | | | | AGGAGAGCGGGCCACCATCAACTGTAAAAGTTCTCAGTCC |
| | | | | | CTGCTGAACAGCAGGACGCCTAAGAATTACCTGGCATGGT |
| | | | | | ACCAACAGAAACCTGGACAGCCGCCTAAGCTGCTCATTTAC |
| | | | | | TGGGCCTCCACACGGAAGAGCGGCGTGCCCGACCGGTTTT |
| | | | | | CCGGGAGCGGCTCCGGCACCGACTTTACCTTGACCATCAG |
| | | | | | TTCCCTGCAGGCAGAAGACGTGGCCGTATACTATTGCAAG |
| | | | | | CAATCTTACAATCTCCTGACATTTGGCGGCGGCACAAAAGT |
| | | | | | GGAGATCAAACTCGAGTCTAGAGGGCCCTTCGAACAAAAA |
| | | | | | CTCATCTCAGAAGAGGATCTGAATATGCATACCGGTCATCA |
| | | | | | TCACCATCACCATTGA (SEQ ID NO: 118) |
| KL2B359LH- | KLK2 | VL-VH | GGGGS (SEQ | La5B9 | MAWVWTLLFLMAAAQSIQAEIVLTQSPATLSLSPGERATLSCR |
| 5B9HL | | scFv | ID NO: 124) | scFv | ASESVEYFGTSLMHWYQQKPGQPPRLLIYAASNVESGIPARF |
| | | | | | SGSGSGTDFTLTISSVEPEDFAVYFCQQTRKVPYTFGGGTKV |
| | | | | | EIKGGSEGKSSGSGSESKSTGGSQVQLQESGPGLVKPSQTL |
| | | | | | SLTCTVSGNSITSDYAWNWIRQFPGKRLEWIGYISYSGSTTYN |
| | | | | | PSLKSRVTISRDTSKNQFSLKLSSVTAADTAVYYCATGYYYGS |
| | | | | | GFWGQGTLVTVSSGGGGSQVQLVQSGAEVKKPGASVKVSC |
| | | | | | KASGYTFTHYYIYWVRQAPGQGLEWMGGVNPSNGGTHFNEK |
| | | | | | FKSRVTMTRDTSISTAYMELSRLRSDDTAVYYCARSEYDYGL |
| | | | | | GFAYWGQGTLVTVSSGGSEGKSSGSGSESKSTGGSDIVMTQ |
| | | | | | SPDSLAVSLGERATINCKSSQSLLNSRTPKNYLAWYQQKPGQ |
| | | | | | PPKLLIYWASTRKSGVPDRFSGSGSGTDFTLTISSLQAEDVAV |
| | | | | | YYCKQSYNLLTFGGGTKVEIKLESRGPFEQKLISEEDLNMHTG |
| | | | | | HHHHHH (SEQ ID NO: 119) |
| | | | | | ATGGCTTGGGTTTGGACCCTGCTGTTCCTGATGGCCGCTG |
| | | | | | CTCAGTCTATCCAGGCTGAGATCGTGCTGACCCAGTCCCCT |
| | | | | | GCCACACTGTCTCTGAGCCCAGGAGAGAGGGCCACCCTGT |
| | | | | | CTTGCAGGGCATCCGAGTCTGTGGAGTATTTCGGCACAAG |
| | | | | | CCTGATGCACTGGTATCAGCAGAAGCCAGGCCAGCCCCCT |
| | | | | | AGGCTGCTGATCTATGCAGCAAGCAACGTGGAGTCCGGCA |
| | | | | | TCCCCGCACGCTTCAGCGGCTCCGGCTCTGGCACCGACTT |
| | | | | | TACCCTGACAATCAGCTCCGTGGAGCCCGAGGATTTCGCC |
| | | | | | GTGTATTTTTGCCAGCAGACACGGAAGGTGCCTTACACCTT |
| | | | | | TGGCGGCGGCACAAAGGTGGAGATCAAGGGAGGCTCCGA |
| | | | | | GGGCAAGTCTAGCGGCAGCGGCTCCGAGTCTAAGAGCACC |
| | | | | | GGAGGCAGCCAGGTGCAGCTGCAGGAGTCCGGACCAGGC |
| | | | | | CTGGTGAAGCCATCTCAGACCCTGAGCCTGACCTGTACAG |
| | | | | | TGTCCGGCAACTCTATCACAAGCGACTATGCCTGGAATTGG |
| | | | | | ATCAGACAGTTCCCTGGCAAGAGACTGGAGTGGATCGGCT |
| | | | | | ATATCTCCTACTCTGGCAGCACCACATACAACCCCTCCCTG |
| | | | | | AAGTCTCGGGTGACCATCTCCAGAGACACATCTAAGAATCA |
| | | | | | GTTCAGCCTGAAGCTGTCCTCTGTGACCGCCGCCGATACA |
| | | | | | GCCGTGTACTATTGTGCCACCGGCTACTATTACGGCTCCG |
| | | | | | GATTTTGGGGACAGGGCACCCTGGTGACAGTGAGCTCCGG |
| | | | | | AGGAGGAGGATCCCAAGTGCAACTGGTCCAGTCAGGTGCT |
| | | | | | GAGGTGAAAAAACCCGGAGCCAGTGTCAAAGTAAGCTGCA |
| | | | | | AGGCCTCTGGGTATACTTTCACCCATTACTATATATACTGG |
| | | | | | GTTCGTCAAGCTCCAGGTCAGGGGCTTGAGTGGATGGGTG |
| | | | | | GAGTCAACCCTTCGAACGGTGGCACTCACTTCAATGAAAAG |
| | | | | | TTTAAAAGCCGCGTAACCATGACGCGAGATACTTCCATTTC |
| | | | | | CACAGCTTATATGGAACTTAGTAGGTTACGCAGTGATGACA |
| | | | | | CGGCCGTTTATTACTGTGCTAGAAGTGAATATGATTATGGG |
| | | | | | TTGGGTTTCGCTTACTGGGGCCAGGGAACCCTCGTCACCG |
| | | | | | TGTCCAGTGGAGGCAGCGAGGGCAAGTCTAGCGGCTCCG |
| | | | | | GCTCTGAGAGCAAGTCCACCGGAGGCAGCGACATTGTTAT |
| | | | | | GACGCAGAGCCCTGATTCACTCGCAGTGTCCCTAGGAGAG |
| | | | | | CGGGCCACCATCAACTGTAAAAGTTCTCAGTCCCTGCTGAA |
| | | | | | CAGCAGGACGCCTAAGAATTACCTGGCATGGTACCAACAG |
| | | | | | AAACCTGGACAGCCGCCTAAGCTGCTCATTTACTGGGCCTC |
| | | | | | CACACGGAAGAGCGGCGTGCCCGACCGGTTTTCCGGGAG |
| | | | | | CGGCTCCGGCACCGACTTTACCTTGACCATCAGTTCCCTGC |
| | | | | | AGGCAGAAGACGTGGCCGTATACTATTGCAAGCAATCTTAC |
| | | | | | AATCTCCTGACATTTGGCGGCGGCACAAAAGTGGAGATCAA |
| | | | | | ACTCGAGTCTAGAGGGCCCTTCGAACAAAAACTCATCTCAG |
| | | | | | AAGAGGATCTGAATATGCATACCGGTCATCATCACCATCAC |
| | | | | | CATTGA (SEQ ID NO: 120) |
| TIMEF9LH- | TMEFF2 | VL-VH | NSGGGGS | La5B9 | MAWVWTLLFLMAAAQSIQASYELTQPASVSGSPGQSITISCIG |
| 5B9HL | | scFv | (SEQ ID NO: | scFv | TSSDVGSYNLVSWYQQHPGKVPKLMIYEGSKRPSGVSNRFS |
| | | | 123) | | GSKSGNTASLTISGLQAEDEADYYCSSYAGSSTYVFGTGTKV |
| | | | | | TVLGGSEGKSSGSGSESKSTGGSQVQLQESGPGLVKPSETL |
| | | | | | SLTCTVSGVSISSYFWSWLRQPAGKGLQWIGRISTSGSTNHN |
| | | | | | PSLKSRVIMSVDTSKNQFSLKLSSVTAADTAVYYCVRDWTGF |
| | | | | | DYWGQGTLVTVSSNSGGGGSQVQLVQSGAEVKKPGASVKV |
| | | | | | SCKASGYTFTHYYIYWVRQAPGQGLEWMGGVNPSNGGTHF |
| | | | | | NEKFKSRVTMTRDTSISTAYMELSRLRSDDTAVYYCARSEYD |
| | | | | | YGLGFAYWGQGTLVTVSSGGSEGKSSGSGSESKSTGGSDIV |
| | | | | | MTQSPDSLAVSLGERATINCKSSQSLLNSRTPKNYLAWYQQK |
| | | | | | PGQPPKLLIYWASTRKSGVPDRFSGSGSGTDFTLTISSLQAED |
| | | | | | VAVYYCKQSYNLLTFGGGTKVEIKLESRGPFEQKLISEEDLNM |
| | | | | | HTGHHHHHH (SEQ ID NO: 121) |
| | | | | | ATGGCCTGGGTGTGGACCCTGCTGTTCCTGATGGCAGCAG |
| | | | | | CACAGAGCATCCAGGCCTCCTACGAGCTGACACAGCCTGC |
| | | | | | ATCTGTGAGCGGCTCCCCAGGACAGTCTATCACCATCAGCT |
| | | | | | GCATCGGCACAAGCTCCGACGTGGGCTCCTACAACCTGGT |
| | | | | | GTCTTGGTATCAGCAGCACCCCGGCAAGGTGCCTAAGCTG |
| | | | | | ATGATCTATGAGGGCAGCAAGAGGCCAAGCGGCGTGTCCA |
| | | | | | ACAGATTCTCTGGCAGCAAGTCCGGCAATACCGCCTCCCT |
| | | | | | GACAATCTCTGGACTGCAGGCAGAGGACGAGGCAGATTAC |
| | | | | | TATTGCTCTAGCTACGCCGGCTCCTCTACCTACGTGTTCGG |
| | | | | | CACCGGCACAAAGGTGACAGTGCTGGGAGGCTCCGAGGG |
| | | | | | CAAGAGCTCCGGCTCTGGCAGCGAGTCCAAGTCTACCGGA |
| | | | | | GGCTCCCAGGTGCAGCTGCAGGAGAGCGGACCAGGACTG |
| | | | | | GTGAAGCCAAGCGAGACACTGTCCCTGACCTGTACAGTGT |
| | | | | | CTGGCGTGAGCATCTCTAGCTACTTTTGGAGCTGGCTGAG |
| | | | | | GCAGCCAGCAGGCAAGGGACTGCAGTGGATCGGCCGCAT |
| | | | | | CAGCACCTCCGGCTCTACAAACCACAATCCTTCCCTGAAGT |
| | | | | | CTAGAGTGATCATGTCTGTGGACACCAGCAAGAACCAGTTC |
| | | | | | TCCCTGAAGCTGTCCTCTGTGACCGCCGCCGATACAGCCG |
| | | | | | TGTACTATTGCGTGCGGGACTGGACCGGCTTTGATTATTGG |
| | | | | | GGCCAGGGCACCCTGGTGACAGTGAGCTCCAATAGCGGC |
| | | | | | GGCGGCGGATCCCAAGTGCAACTGGTCCAGTCAGGTGCTG |
| | | | | | AGGTGAAAAAACCCGGAGCCAGTGTCAAAGTAAGCTGCAA |
| | | | | | GGCCTCTGGGTATACTTTCACCCATTACTATATATACTGGGT |
| | | | | | TCGTCAAGCTCCAGGTCAGGGGCTTGAGTGGATGGGTGGA |
| | | | | | GTCAACCCTTCGAACGGTGGCACTCACTTCAATGAAAAGTT |
| | | | | | TAAAAGCCGCGTAACCATGACGCGAGATACTTCCATTTCCA |
| | | | | | CAGCTTATATGGAACTTAGTAGGTTACGCAGTGATGACACG |
| | | | | | GCCGTTTATTACTGTGCTAGAAGTGAATATGATTATGGGTT |
| | | | | | GGGTTTCGCTTACTGGGGCCAGGGAACCCTCGTCACCGTG |
| | | | | | TCCAGTGGAGGCAGCGAGGGCAAGTCTAGCGGCTCCGGC |
| | | | | | TCTGAGAGCAAGTCCACCGGAGGCAGCGACATTGTTATGA |
| | | | | | CGCAGAGCCCTGATTCACTCGCAGTGTCCCTAGGAGAGCG |
| | | | | | GGCCACCATCAACTGTAAAAGTTCTCAGTCCCTGCTGAACA |
| | | | | | GCAGGACGCCTAAGAATTACCTGGCATGGTACCAACAGAA |
| | | | | | ACCTGGACAGCCGCCTAAGCTGCTCATTTACTGGGCCTCC |
| | | | | | ACACGGAAGAGCGGCGTGCCCGACCGGTTTTCCGGGAGC |
| | | | | | GGCTCCGGCACCGACTTTACCTTGACCATCAGTTCCCTGCA |
| | | | | | GGCAGAAGACGTGGCCGTATACTATTGCAAGCAATCTTACA |
| | | | | | ATCTCCTGACATTTGGCGGCGGCACAAAAGTGGAGATCAAA |
| | | | | | CTCGAGTCTAGAGGGCCCTTCGAACAAAAACTCATCTCAGA |
| | | | | | AGAGGATCTGAATATGCATACCGGTCATCATCACCATCACC |
| | | | | | ATTGA (SEQ ID NO: 122) |

It is claimed:

1. A method of retargeting a recombinant herpes simplex virus (HSV) to a tumor cell expressing a TAA, the method comprising administering to a subject having the tumor cell,
(a) the recombinant HSV, wherein the recombinant HSV comprises a nucleotide sequence encoding a heterologous ligand peptide; and
(b) an isolated bispecific adaptor protein, wherein the bispecific adaptor protein comprises a first binding domain with binding specificity to the heterologous ligand peptide expressed by the recombinant HSV and a second binding domain with binding specificity to the TAA expressed by the tumor cell,
wherein, the first binding domain of the bispecific adaptor protein binds the heterologous ligand peptide expressed by the recombinant HSV and the second binding domain of the bispecific adaptor protein binds the TAA expressed by the tumor cell, thereby retargeting the recombinant HSV to the tumor cell.

2. The method of claim 1, wherein the nucleotide sequence encoding the heterologous ligand peptide is inserted into the recombinant HSV by inserting into or replacing a portion of the nucleotide sequence encoding the wild type glycoprotein D (gD).

3. The method of claim 2, wherein the nucleotide sequence encoding the heterologous ligand peptide is inserted into the recombinant HSV replacing a nucleotide sequence encoding the amino acids 6-38 of the wild type glycoprotein D (gD).

4. The method of claim 1-3, wherein the first binding domain of the bispecific adaptor protein comprises an antigen binding fragment with binding specificity to the heterologous ligand peptide expressed by the recombinant HSV.

5. The method of claim 4, wherein the antigen binding fragment with binding specificity to the heterologous ligand peptide is selected from the group consisting of single chain variable region (scFv), single chain antibody VHH, and polypeptide DARPin.

6. The method of claim 1-3, wherein the second binding domain of the bispecific adaptor protein comprises an antigen binding fragment with binding specificity to the TAA expressed by the tumor cell.

7. The method of claim 6, wherein the antigen binding fragment with binding specificity to the TAA is selected from the group consisting of scFv, single chain antibody VHH, polypeptide DARPin.

8. The method of any one of claims 1-7, wherein the heterologous ligand peptide expressed by the recombinant HSV comprises GCN4 transcription factor or a fragment thereof.

9. The method of claim 8, wherein the GCN4 transcription factor or fragment thereof comprises the amino acid sequence of SEQ ID NO: 4.

10. The method of claim 8 or 9, wherein the first binding domain of the bispecific adaptor protein comprises an antigen binding fragment with binding specificity to the GCN4 transcription factor or a fragment thereof.

11. The method of claim 10, wherein the antigen binding fragment with binding specificity to the GCN4 transcription factor or a fragment thereof is an anti-GCN4 scFv comprising a heavy chain variable region (VH) comprised of HCDR1 (SEQ ID NO: 16), HCDR2 (SEQ ID NO: 17), and HCDR3 (SEQ ID NO: 18) and/or a light chain variable region (VL) comprised of LCDR1 (SEQ ID NO: 19), LCDR2 (SEQ ID NO: 20), and LCDR3 (SEQ ID NO: 21).

12. The method of claim 10, wherein the antigen binding fragment with binding specificity to the GCN4 transcription factor or a fragment thereof is an anti-GCN4 scFv comprising a VH having a polypeptide sequence at least 95%, or at least 96%, or at least 97%, or at least 98%, or at least 99%, or 100% identical to SEQ ID NO: 22 and/or a VL having a polypeptide sequence at least 95%, or at least 96%, or at least 97%, or at least 98%, or at least 99%, or 100% identical to SEQ ID NO: 23.

13. The method of any one of claims 1-7, wherein the heterologous ligand peptide expressed by the recombinant HSV comprises La protein or a fragment thereof.

14. The method of claim 13, wherein the La protein or fragment thereof comprises the amino acid sequence of SEQ ID NO: 12.

15. The method of claim 13 or 14, wherein the first binding domain of the bispecific adaptor protein comprises an antigen binding fragment with binding specificity to the La protein or fragment thereof.

16. The method of claim 15, wherein the antigen binding fragment with binding specificity to the La protein or fragment thereof is an anti-La scFv comprising a VH comprised of HCDR1 (SEQ ID NO: 26), HCDR2 (SEQ ID NO: 27), and HCDR3 (SEQ ID NO: 28) and/or a VL comprised of LCDR1 (SEQ ID NO: 29), LCDR2 (SEQ ID NO: 30), and LCDR3 (SEQ ID NO: 31).

17. The method of claim 15, wherein the antigen binding fragment with binding specificity to the La protein or fragment thereof is an anti-La scFv comprising a VH having a polypeptide sequence at least 95%, or at least 96%, or at least 97%, or at least 98%, or at least 99%, or 100% identical to SEQ ID NO: 32 and/or a VL having a polypeptide sequence at least 95%, or at least 96%, or at least 97%, or at least 98%, or at least 99%, or 100% identical to SEQ ID NO: 33.

18. The method of claim 1-3, wherein the heterologous ligand peptide expressed by the recombinant HSV comprises a first leucine-zipper moiety and the first binding domain of the bispecific adaptor protein comprises a second leucine-zipper moiety, wherein the first and second leucine-zipper moieties can form a leucine-zipper dimer.

19. The method of claim 18, wherein the first leucine-zipper moiety is synthetic leucine-zipper moiety RE (SEQ ID NO: 6) and the second leucine-zipper moiety is synthetic leucine-zipper moiety ER (SEQ ID NO: 10), or the first leucine-zipper moiety is synthetic leucine-zipper moiety ER (SEQ ID NO: 10) and the second leucine-zipper moiety is synthetic leucine-zipper moiety RE (SEQ ID NO: 6).

20. The method of any one of claims 1-19, wherein, the TAA expressed by the tumor cell is selected from the group consisting of PSMA, TMEFF2, ROR1, KLK2, and HLA-G.

21. The method of claim 20, wherein the TAA expressed by the tumor cell is PSMA, and wherein the second binding domain of the bispecific adaptor protein comprises an antigen binding fragment with binding specificity to PSMA.

22. The method of claim 21, wherein the antigen binding fragment with binding specificity to PSMA is an anti-PSMA VHH comprising HCDR1 (SEQ ID NO: 35), HCDR2 (SEQ ID NO: 36), and HCDR3 (SEQ ID NO: 37).

23. The method of claim 21, wherein the antigen binding fragment with binding specificity to PSMA is an anti-PSMA VHH comprising a polypeptide sequence at least 95%, or at least 96%, or at least 97%, or at least 98%, or at least 99%, or 100% identical to SEQ ID NO: 38.

24. The method of claim 21, wherein the antigen binding fragment with binding specificity to PSMA is an anti-PSMA VHH comprising HCDR1 (SEQ ID NO: 39), HCDR2 (SEQ ID NO: 40), and HCDR3 (SEQ ID NO: 41).

25. The method of claim 21, wherein the antigen binding fragment with binding specificity to PSMA is an anti-PSMA VHH comprising a polypeptide sequence at least 95%, or at least 96%, or at least 97%, or at least 98%, or at least 99%, or 100% identical to SEQ ID NO: 42.

26. The method of claim 21, wherein the antigen binding fragment with binding specificity to PSMA is an anti-PSMA scFv comprising a VH comprised of HCDR1 (SEQ ID NO: 43), HCDR2 (SEQ ID NO: 44), and HCDR3 (SEQ ID NO: 45) and/or a VL comprised of LCDR1 (SEQ ID NO: 46), LCDR2 (SEQ ID NO: 47), and LCDR3 (SEQ ID NO: 48).

27. The method of claim 21, wherein the antigen binding fragment with binding specificity to PSMA is an anti-PSMA scFv comprising a VH having a polypeptide sequence at least 95%, or at least 96%, or at least 97%, or at least 98%, or at least 99%, or 100% identical to SEQ ID NO: 49 and/or a VL having a polypeptide sequence at least 95%, or at least 96%, or at least 97%, or at least 98%, or at least 99%, or 100% identical to SEQ ID NO: 50.

28. The method of claim 21, wherein the TAA expressed by the tumor cell is TMEFF2, and wherein the second binding domain of the bispecific adaptor protein comprises an antigen binding fragment with binding specificity to TMEFF2.

29. The method of claim 28, wherein the antigen binding fragment with binding specificity to TMEFF2 is an anti-TMEFF2 scFv comprising a VH comprised of HCDR1 (SEQ ID NO: 53), HCDR2 (SEQ ID NO: 54), and HCDR3 (SEQ ID NO: 55) and/or a VL comprised of LCDR1 (SEQ ID NO: 56), LCDR2 (SEQ ID NO: 57), and LCDR3 (SEQ ID NO: 58).

30. The method of claim 28, wherein the antigen binding fragment with binding specificity to TMEFF2 is an anti-TMEFF2 scFv comprising a VH having a polypeptide sequence at least 95%, or at least 96%, or at least 97%, or at least 98%, or at least 99%, or 100% identical to SEQ ID NO: 59 and/or a VL having a polypeptide sequence at least 95%, or at least 96%, or at least 97%, or at least 98%, or at least 99%, or 100% identical to SEQ ID NO: 60.

31. The method of claim 28, wherein the antigen binding fragment with binding specificity to TMEFF2 is an anti-TMEFF2 scFv comprising a VH comprised of HCDR1 (SEQ ID NO: 61), HCDR2 (SEQ ID NO: 62), and HCDR3 (SEQ ID NO: 63) and/or a VL comprised of LCDR1 (SEQ ID NO: 64), LCDR2 (SEQ ID NO: 65), and LCDR3 (SEQ ID NO: 66).

32. The method of claim 28, wherein the antigen binding fragment with binding specificity to TMEFF2 is an anti-TMEFF2 scFv comprising a VH having a polypeptide sequence at least 95%, or at least 96%, or at least 97%, or at least 98%, or at least 99%, or 100% identical to SEQ ID NO: 67 and/or a VL having a polypeptide sequence at least 95%, or at least 96%, or at least 97%, or at least 98%, or at least 99%, or 100% identical to SEQ ID NO: 68.

33. The method of claim 20, wherein the TAA expressed by the tumor cell is KLK2, and wherein the second binding domain of the bispecific adaptor protein comprises an antigen binding fragment with binding specificity to KLK2.

34. The method of claim 33, wherein the antigen binding fragment with binding specificity to KLK2 is an anti-KLK2 scFv comprising a VH comprised of HCDR1 (SEQ ID NO: 72), HCDR2 (SEQ ID NO: 73), and HCDR3 (SEQ ID NO: 74) and/or a VL comprised of LCDR1 (SEQ ID NO: 75), LCDR2 (SEQ ID NO: 76), and LCDR3 (SEQ ID NO: 77).

35. The method of claim 33, wherein the antigen binding fragment with binding specificity to KLK2 is an anti-KLK2 scFv comprising a VH having a polypeptide sequence at least 95%, or at least 96%, or at least 97%, or at least 98%, or at least 99%, or 100% identical to SEQ ID NO: 78 and/or a VL having a polypeptide sequence at least 95%, or at least 96%, or at least 97%, or at least 98%, or at least 99%, or 100% identical to SEQ ID NO: 79.

36. The method of claim 33, wherein the antigen binding fragment with binding specificity to KLK2 is an anti-KLK2 scFv comprises a VH having a polypeptide sequence at least 95%, or at least 96%, or at least 97%, or at least 98%, or at least 99%, or 100% identical to SEQ ID NO: 80 and/or a VL having a polypeptide sequence at least 95%, or at least 96%, or at least 97%, or at least 98%, or at least 99%, or 100% identical to SEQ ID NO: 81.

37. The method of claim 20, wherein the TAA expressed by the tumor cell is HLA-G, and wherein the second binding domain of the bispecific adaptor protein comprises an antigen binding fragment with binding specificity to HLA-G.

38. The method of claim 20, wherein the TAA expressed by the tumor cell is ROR1, and wherein the second binding domain of the bispecific adaptor protein comprises an antigen binding fragment with binding specificity to ROR1.

39. The method of claim 38, wherein the antigen binding fragment with binding specificity to ROR1 is a polypeptide DARPin having a polypeptide sequence at least 95%, or at least 96%, or at least 97%, or at least 98%, or at least 99%, or 100% identical to SEQ ID NO: 94.

40. A method of treating a cancer in a subject, wherein a TAA is expressed by the cancer cell, the method comprising administering to the subject,
(a) a recombinant HSV, wherein the recombinant HSV comprises a nucleotide sequence encoding a heterologous ligand peptide; and
(b) an isolated bispecific adaptor protein, wherein the bispecific adaptor protein comprises a first binding domain with binding specificity to the heterologous ligand peptide expressed by the recombinant HSV and a second binding domain with binding specificity to the TAA expressed by the cancer cell,
wherein, the first binding domain of the bispecific adaptor protein binds the heterologous ligand peptide expressed by the recombinant HSV, the second binding domain of the specific adaptor protein binds the TAA expressed by the cancer cell, and thereby causing oncolysis of the cancer cell.

41. The method of claim 40, wherein the nucleotide sequence encoding the heterologous ligand peptide is inserted into the recombinant HSV by inserting into or replacing a portion of the nucleotide sequence encoding the wild type glycoprotein D (gD).

42. The method of claim 40 or 41, wherein the nucleotide sequence encoding the heterologous ligand peptide is inserted into the recombinant HSV replacing a nucleotide sequence encoding the amino acids 6-38 of wild type gD.

43. A bispecific adaptor protein for retargeting a recombinant HSV to a tumor cell, wherein the bispecific adaptor protein comprises a first binding domain with binding specificity to a heterologous ligand peptide expressed by the recombinant HSV and a second binding domain with binding specificity to a TAA expressed by the tumor cell.

44. The bispecific adaptor protein of claim 43, wherein each of the first and second binding domains of the bispecific adaptor protein comprises an antigen binding fragment.

45. The bispecific adaptor protein of claim 44, wherein the antigen binding fragment is selected from the group consisting of scFv, single chain antibody VHH, and polypeptide DARPin.

46. The bispecific adaptor protein of any one of claims 43-45, wherein the first binding domain of the bispecific adaptor protein comprises an antigen binding fragment with binding specificity to GCN4 transcription factor or a fragment thereof.

47. The bispecific adaptor protein of claim 46, wherein the antigen binding fragment with binding specificity to GCN4 transcription factor or a fragment thereof is an anti-GCN4 scFv comprising a VH comprised of HCDR1 (SEQ ID NO: 16), HCDR2 (SEQ ID NO: 17), and HCDR3 (SEQ ID NO: 18) and/or a VL comprised of LCDR1 (SEQ ID NO: 19), LCDR2 (SEQ ID NO: 20), and LCDR3 (SEQ ID NO: 21).

48. The bispecific adaptor protein of claim 46, wherein the antigen binding fragment with binding specificity to GCN4 transcription factor or a fragment thereof is an anti-GCN4 scFv comprising a VH having a polypeptide sequence at least 95%, or at least 96%, or at least 97%, or at least 98%, or at least 99%, or 100% identical to SEQ ID NO: 22 and/or a VL having a polypeptide sequence at least 95%, or at least 96%, or at least 97%, or at least 98%, or at least 99%, or 100% identical to SEQ ID NO: 23.

49. The bispecific adaptor protein of any one of claims 43-45, wherein the first binding domain of the bispecific adaptor protein comprises an antigen binding fragment with binding specificity to La protein or fragment thereof.

50. The bispecific adaptor protein of claim 49, wherein the antigen binding fragment with binding specificity to La protein or fragment thereof is an anti-La scFv comprising a VH comprised of HCDR1 (SEQ ID NO: 26), HCDR2 (SEQ ID NO: 27), and HCDR3 (SEQ ID NO: 28) and/or a VL comprised of LCDR1 (SEQ ID NO: 29), LCDR2 (SEQ ID NO: 30), and LCDR3 (SEQ ID NO: 31).

51. The bispecific adaptor protein of claim 49, wherein the antigen binding fragment with binding specificity to La protein or fragment thereof is an anti-La scFv comprising a VH having a polypeptide sequence at least 95%, or at least 96%, or at least 97%, or at least 98%, or at least 99%, or 100% identical to SEQ ID NO: 32 and/or a VL having a polypeptide sequence at least 95%, or at least 96%, or at least 97%, or at least 98%, or at least 99%, or 100% identical to SEQ ID NO: 33.

52. The bispecific adaptor protein of claim 43, wherein the first binding domain of the bispecific adaptor protein comprises a leucine-zipper moiety.

53. The bispecific adaptor protein of claim 52, wherein the leucine-zipper moiety is synthetic leucine-zipper moiety RE (SEQ ID NO: 6) or synthetic leucine-zipper moiety ER (SEQ ID NO: 10).

54. The bispecific adaptor protein of any one of claims 43-53, wherein the TAA expressed by the tumor cell is PSMA, and wherein the second binding domain of the bispecific adaptor protein comprises an antigen binding fragment with binding specificity to PSMA.

55. The bispecific adaptor protein of claim 54, wherein the antigen binding fragment with binding specificity to PSMA is an anti-PSMA VHH comprising HCDR1 (SEQ ID NO: 35), HCDR2 (SEQ ID NO: 36), and HCDR3 (SEQ ID NO: 37).

56. The bispecific adaptor protein of claim 54, wherein the antigen binding fragment with binding specificity to PSMA is an anti-PSMA VHH comprising a polypeptide sequence at least 95%, or at least 96%, or at least 97%, or at least 98%, or at least 99%, or 100% identical to SEQ ID NO: 38.

57. The bispecific adaptor protein of claim 54, wherein the antigen binding fragment with binding specificity to PSMA is an anti-PSMA VHH comprising HCDR1 (SEQ ID NO: 39), HCDR2 (SEQ ID NO: 40), and HCDR3 (SEQ ID NO: 41).

58. The bispecific adaptor protein of claim 54, wherein the antigen binding fragment with binding specificity to PSMA is an anti-PSMA VHH comprising a polypeptide sequence at least 95%, or at least 96%, or at least 97%, or at least 98%, or at least 99%, or 100% identical to SEQ ID NO: 42.

59. The bispecific adaptor protein of claim 54, wherein the antigen binding fragment with binding specificity to PSMA an anti-PSMA scFv comprising a VH comprised of HCDR1 (SEQ ID NO: 43), HCDR2 (SEQ ID NO: 44), and HCDR3 (SEQ ID NO: 45) and/or a VL comprised of LCDR1 (SEQ ID NO: 46), LCDR2 (SEQ ID NO: 47), and LCDR3 (SEQ ID NO: 48).

60. The bispecific adaptor protein of claim 54, wherein the antigen binding fragment with binding specificity to PSMA is an anti-PSMA scFv comprising a VH having a polypeptide sequence at least 95%, or at least 96%, or at least 97%, or at least 98%, or at least 99%, or 100% identical to SEQ ID NO: 49 and/or a VL having a polypeptide sequence at least 95%, or at least 96%, or at least 97%, or at least 98%, or at least 99%, or 100% identical to SEQ ID NO: 50.

61. The bispecific adaptor protein of any one of claims 43-53, wherein the TAA expressed by the tumor cell is TMEFF2, and wherein the second binding domain of the bispecific adaptor protein comprises an antigen binding fragment with binding specificity to TMEFF2.

62. The bispecific adaptor protein of claim 61, wherein the antigen binding fragment with binding specificity to TMEFF2 is anti-TMEFF2 scFv comprising a VH comprised of HCDR1 (SEQ ID NO: 53), HCDR2 (SEQ ID NO: 54), and HCDR3 (SEQ ID NO: 55) and/or a VL comprised of LCDR1 (SEQ ID NO: 56), LCDR2 (SEQ ID NO: 57), and LCDR3 (SEQ ID NO: 58).

63. The bispecific adaptor protein of claim 61, wherein the antigen binding fragment with binding specificity to TMEFF2 is an anti-TMEFF2 scFv comprising a VH having a polypeptide sequence at least 95%, or at least 96%, or at least 97%, or at least 98%, or at least 99%, or 100% identical to SEQ ID NO: 59 and/or a VL having a polypeptide sequence at least 95%, or at least 96%, or at least 97%, or at least 98%, or at least 99%, or 100% identical to SEQ ID NO: 60.

64. The bispecific adaptor protein of claim 61, wherein the antigen binding fragment with binding specificity to TMEFF2 is an anti-TMEFF2 scFv comprising a VH comprised of HCDR1 (SEQ ID NO: 61), HCDR2 (SEQ ID NO: 62), and HCDR3 (SEQ ID NO: 63) and/or a VL comprised of LCDR1 (SEQ ID NO: 64), LCDR2 (SEQ ID NO: 65), and LCDR3 (SEQ ID NO: 66).

65. The bispecific adaptor protein of claim 61, wherein the antigen binding fragment with binding specificity to TMEFF2 is an anti-TMEFF2 scFv comprising a VH having a polypeptide sequence at least 95%, or at least 96%, or at least 97%, or at least 98%, or at least 99%, or 100% identical to SEQ ID NO: 67 and/or a VL having a polypeptide sequence at least 95%, or at least 96%, or at least 97%, or at least 98%, or at least 99%, or 100% identical to SEQ ID NO: 68.

66. The bispecific adaptor protein of any one of claims 43-53, wherein the TAA expressed by the tumor cell is KLK2, and wherein the second binding domain of the bispecific adaptor protein comprises an antigen binding fragment with binding specificity to KLK2.

67. The bispecific adaptor protein of claim 66, wherein the antigen binding fragment with binding specificity to KLK2 is an anti-KLK2 scFv comprising a VH comprised of HCDR1 (SEQ ID NO: 72), HCDR2 (SEQ ID NO: 73), and HCDR3 (SEQ ID NO: 74) and/or a VL comprised of LCDR1 (SEQ ID NO: 75), LCDR2 (SEQ ID NO: 76), and LCDR3 (SEQ ID NO: 77).

68. The bispecific adaptor protein of claim 66, wherein the antigen binding fragment with binding specificity to KLK2 is anti-KLK2 scFv comprising a VH having a polypeptide sequence at least 95%, or at least 96%, or at least 97%, or at least 98%, or at least 99%, or 100% identical to SEQ ID NO: 78 and/or a VL having a polypeptide sequence at least 95%, or at least 96%, or at least 97%, or at least 98%, or at least 99%, or 100% identical to SEQ ID NO: 79.

69. The bispecific adaptor protein of claim 66, wherein the antigen binding fragment with binding specificity to KLK2 is an anti-KLK2 scFv comprising a VH having a polypeptide sequence at least 95%, or at least 96%, or at least 97%, or at least 98%, or at least 99%, or 100% identical to SEQ ID NO: 80 and/or a VL having a polypeptide sequence at least 95%, or at least 96%, or at least 97%, or at least 98%, or at least 99%, or 100% identical to SEQ ID NO: 81.

70. The bispecific adaptor protein of any one of claims 43-53, wherein the TAA expressed by the tumor cell is HLA-G, and wherein the second binding domain of the bispecific adaptor protein comprises an antigen binding fragment with binding specificity to HLA-G.

71. The bispecific adaptor protein of any one of claims 43-53, wherein the TAA expressed by the tumor cell is ROR1, and wherein the second binding domain of the bispecific adaptor protein comprises an antigen binding fragment with binding specificity to ROR1.

72. The bispecific adaptor protein of claim 71, wherein the antigen binding fragment with binding specificity to ROR1 is a polypeptide DARPin having a polypeptide sequence at least 95%, or at least 96%, or at least 97%, or at least 98%, or at least 99%, or 100% identical to SEQ ID NO: 94.

73. An isolated nucleic acid comprising a polynucleotide sequence encoding the isolated bispecific adaptor protein of any one of claims 43-71.

74. An isolated vector comprising the isolated nucleic acid sequence of claim 73.

75. A recombinant host cell comprising the isolated vector of claim 74.

76. A kit comprising a recombinant HSV as described in any one of claims 1-39 and instructions for use of the recombinant HSV.

77. A kit comprising an isolated bispecific adaptor protein of any one of claims 43-72 and instructions for use of the bispecific adaptor protein.

78. A kit comprising a recombinant HSV as described in any one of claims 1-93, an isolated adaptor protein of any one of claims 43-72, and instructions for use.

79. A recombinant HSV comprising a nucleotide sequence encoding a heterologous ligand peptide, wherein the heterologous ligand peptide comprises La protein or a fragment thereof, and wherein the nucleotide sequence encoding the heterologous ligand peptide is inserted into the recombinant HSV by inserting into or replacing a portion of replacing the wild type gD.

80. The recombinant HSV of claim 79, wherein the nucleotide sequence encoding the heterologous ligand peptide is inserted into the recombinant HSV replacing a nucleotide sequence encoding the amino acid 6-38 of wild type gD.

81. The recombinant HSV of claim 79 or 80, wherein the La protein or fragment thereof comprises the amino acid sequence of SEQ ID NO: 12.

82. A recombinant HSV comprising a nucleotide sequence encoding a heterologous ligand peptide, wherein the heterologous ligand peptide comprises a leucine-zipper moiety, and wherein the nucleotide sequence encoding the heterologous ligand peptide is inserted into the recombinant HSV by inserting into or replacing a portion the wild type gD.

83. The recombinant HSV of claim 82, wherein the nucleotide sequence encoding the heterologous ligand peptide is inserted into the recombinant HSV replacing a nucleotide sequence encoding the amino acid 6-38 of wild type gD.

84. The recombinant HSV of claim 83, wherein the leucine-zipper moiety is synthetic leucine-zipper moiety RE (SEQ ID NO: 6) or synthetic leucine-zipper moiety ER (SEQ ID NO: 10).
